(19) United States
(12) Patent Application Publication
(10) Pub. No.: US 2025/0201411 A1
(43) Pub. Date: Jun. 19, 2025

(54) Machine Learning (ML)-Based Disease-Detection System Using Detection Animals

(71) Applicant: Spotitearly Ltd., Hama'apil (IL)

(72) Inventors: Roi Ophir, Tel Aviv (IL); Ohad Sharon, Hamaapil (IL); Assaf Rabinowicz, Bat-Yam (IL); Udi Bobrovsky, Saad (IL); Reef Einoch Amor, YoKne'am Illit (IL); Amir Lifshitz, Kibutz Hama'apil (IL); Irit Gazit, Ram-on (IL)

(21) Appl. No.: 18/979,459

(22) Filed: Dec. 12, 2024

Related U.S. Application Data
(60) Provisional application No. 63/609,802, filed on Dec. 13, 2023.

(51) Int. Cl. G16H 50/20; A61B 5/0205; G06N 20/00

(52) U.S. Cl. CPC G16H 50/20; A61B 5/02055; G06N 20/00

(57) ABSTRACT

Described herein are systems for disease detection from a biological sample using a machine learning-based (ML-based) disease-detection model trained on a dataset of detection events. Also described are methods for detecting a disease category from a biological sample received from a subject, and further detecting the specific disease type within the disease category using the systems and ML-based disease detection models. Also described are methods for monitoring progression of a disease in a subject by analyzing a biological sample using the systems and ML-based disease-detection model disclosed herein.

### PRIORITY

This application claims the benefit under 35 U.S.C. 119 (e) to U.S. Provisional Patent Application No. 63/609,802, filed 13 Dec. 2023, which is incorporated herein by reference.

### TECHNICAL FIELD

This disclosure relates generally to medical diagnostics using a system of signal analysis of detection animals.

### BACKGROUND

People undergo medical screening for a variety of reasons, including screening tests as part of a general health screening or diagnostic tests to detect for specific conditions. For many diseases, patient outcomes improve significantly if the disease is detected early. Accordingly, there are many screening and diagnostic tests which attempt to detect diseases such as various cancers, heart disease, and other medical conditions. Presently, most diagnostic tests require point-of-care visits, which require a patient to be at a medical facility. Alternatively, some tests require a professional nurse to travel to a patient's home to take measurements. Furthermore, most tests are performed due to a patient exhibiting a particular symptom, and most tests scan for a specific outcome. Due to the above, screening and diagnostic tests today are expensive, intrusive, lack test sensitivity, and require a large time commitment by both the healthcare system and the patient. As a result, adherence to various tests and the willingness of people to adopt them is not high.

There are many screening and diagnostic tests for cancer detection. Example tests include liquid biopsy, which is not only expensive and requires point-of-care specimen collection, but also has low sensitivity to detecting cancer at its early stages. Another example cancer detection procedure is by nematode-based multi-cancer early detection (N-NOSE), which is performed by collecting a patient's urine sample. Many cancer screens detect a limited number of types of cancer and require a separate screening procedure for each cancer. These cancer screens are expensive, inconvenient, invasive, and require point-of-care settings which require a substantial time commitment. Further, these cancer screens lack sensitivity or result in high false positive rates. Moreover, laboratories have a limited capacity to perform these tests and patients have a low adherence rate in properly preparing for these tests. Thus, there is a need for medical diagnostic and screen tests which have high sensitivity, are non-invasive, non-expensive, efficient, and capable of detecting many different diseases.

Many physiological processes, including diseases such as cancer, produce detectable odorants which animals may be trained to detect. For example, cancers produce volatile organic compounds (VOCs) which are excreted into the blood, sweat, saliva, urine, and breath of people with cancer. VOCs are a crucial, early indication of cancer. Traditional diagnostic devices are unable to perform cancer detection using VOC monitoring due, in part, to the low concentrations of cancerous VOCs and a low signal-to-noise ratio. However, VOCs produce a distinctive odor profile which are detectable by canines and other animals. Further, different types of cancer have unique VOC signatures which may be identified by trained animals.

Additionally, certain bacterial or viral infections produce unique scent profiles in living organisms such as humans and animals. These odorants are typically released from humans through breath, urine, feces, skin emanations, and blood, and may be detectable by animals with strong olfactory abilities.

Canines have extremely sensitive olfactory receptors and are able to detect many scents that a human cannot. Canines can pick out specific scent molecules in the air, even at low concentrations. Further, canines may be trained (i.e., conditioned) to perform a certain act, such as sitting down, upon detection of a target odor. Similar to canines, rodents, fruit flies, and bees also have high olfactory capabilities and may be trained to detect specific scents.

### SUMMARY OF PARTICULAR EMBODIMENTS

Certain technical challenges exist for detection of diseases including cancers. One technical challenge with current disease detection methods like biopsy may include the poor sensitivity or specificity for early detection including a low signal-to-noise ratio. The solution presented by the embodiments disclosed herein to address this challenge may be use of systems and methods that employ machine learning-based (ML-based) disease-detection model. Another technical challenge may include the need for invasive detection methods including biopsy, or the burden of point-of-care visits that a patient must undertake for screening and diagnosis. The solution presented by the embodiments disclosed herein to address this challenge may be the use of novel sample collection devices and methods supported by ML-based disease-detection models and systems.

The presently disclosed subject matter is directed to systems and methods for disease detection of a patient sample using machine learning-based (ML-based) disease-detection models trained on a dataset of detection events. The presently disclosed subject matter is also directed to systems and methods for identifying one or more disease types and monitor disease progression in the subject using the ML-based disease-detection models. Certain embodiments disclosed herein may provide one or more technical advantages. A technical advantage of the embodiments may include high sensitivity. Another technical advantage of the embodiments may include high specificity. Yet another technical advantage of the embodiments may include a high signal-to-noise ratio. Certain embodiments disclosed herein may provide none, some, or all of the above technical advantages. One or more other technical advantages may be readily apparent to one skilled in the art in view of the figures, descriptions, and claims of the present disclosure. Certain embodiments disclosed herein may provide further advantages, such as lower costs than existing diagnostic methods.

In particular embodiments, the techniques described herein relate to a system for disease detection including: one or more machine learning-based (ML-based) disease-detection models trained on a dataset of detection events, wherein the models are operable to: receive a first sensor data associated with a first set of detection animals that have been exposed to a biological sample of a patient; calculate, based on the first sensor data, a first confidence score corresponding to a disease category associated with the biological sample, wherein the disease category includes a plurality of disease states, and wherein the first confidence score indicates a likelihood of at least one of the disease states of the disease category being present in the patient; responsive to the first confidence score being greater than a first threshold score, receive a second sensor data associated with a second set of detection animals that have been exposed to the biological sample of the patient; and calculate, based on the second sensor data, one or more second confidence scores corresponding to one or more disease states in the disease category associated with the biological sample, wherein each confidence score indicates a likelihood of a respective disease state in the disease category being present in the patient.

In particular embodiments, the techniques described herein relate to a system, wherein: the first sensor data includes data associated with a conditioned response of the first set of detection animals; and the second sensor data includes data associated with a conditioned response of the second set of detection animals.

In particular embodiments, the techniques described herein relate to a system, wherein the first sensor data and the second sensor data include data received from one or more of: one or more behavioral sensors, one or more physiological sensors, or one or more environmental sensors.

In particular embodiments, the techniques described herein relate to a system, wherein the one or more behavioral sensors of the detection animal includes one or more of: a face gesture of the detection animal, tail movements of the detection animal, landmarks on a skeleton model of the detection animal a duration of a sniff from the detection animal, a sniff intensity, a number of repeated sniffs, a pose of the detection animal, whether the detection animal looks at its handler, a pressure of a nose of the detection animal against a sampling port, or auditory features of the sniff.

In particular embodiments, the techniques described herein relate to a system, wherein the one or more physiological sensors includes one or more of: one or more heart rate sensors, one or more heart rate variability sensors, one or more temperature sensors, one or more breath rate sensors, one or more sweat rate sensors, one or more blood pressure sensors, one or more skin temperature sensors, one or more pupil size variability sensors, one or more salivary cortisol sensors, one or more galvanic skin response (GSR) sensors, one or more electroencephalogram (EEG) sensors, one or more functional near-infrared spectroscopy (fNIR) sensors, one or more functional magnetic resonance imaging (fMRI) scanners, one or more electromyography imaging (EMG) scanners, or one or more magnetic resonance imaging (MRI) scanners.

In particular embodiments, the techniques described herein relate to a system, wherein the one or more environmental sensors include one or more of: one or more temperature sensors, one or more humidity sensors, one or more audio sensors, one or more gas sensors, or one or more air particulate sensors.

In particular embodiments, the techniques described herein relate to a system, wherein: the first set of detection animals is conditioned to detect the disease category of the biological sample; and the second set of detection animals is conditioned to detect the disease state of the biological sample.

In particular embodiments, the techniques described herein relate to a system, wherein: the system further includes one or more breath sensors; and the models are further operable to: detect volatile organic compounds (VOCs) in the biological sample from the one or more breath sensors, wherein presence of the VOCs validates the biological sample as containing biological material from the patient.

In particular embodiments, the techniques described herein relate to a system, wherein the one or more breath sensors are selected from the group including: TVOC sensor, breath VOC sensor, relative humidity sensors, temperature sensor, photoionization detector (PID), flame ionization detector (FID), and metal oxide (MOX) sensor.

In particular embodiments, the techniques described herein relate to a system, wherein the models are further operable to: receive a third sensor data associated with the first set of detection animals or the second set of detection animals that have been exposed to one or more of a service sample, and calculate, based on the third sensor data, one or more confidence scores, each corresponding to a positive control category or a negative control category.

In particular embodiments, the techniques described herein relate to a system, wherein the first and second sets of detection animals are exposed to each of the biological sample and the service sample via a sampling port.

In particular embodiments, the techniques described herein relate to a system, wherein the sampling port is fluidly connected to one or more receptacles of a plurality of receptacles, each receptacle operable to hold the biological sample or the service sample.

In particular embodiments, the techniques described herein relate to a system, wherein the models are further operable to determine which of a particular sample to expose to the first set of detection animals or the second set of detection animals, wherein the particular sample is selected from a group consisting of: the biological sample from the patient and the service sample.

In particular embodiments, the techniques described herein relate to a system, wherein the models are further operable to: identify the biological sample as associated with at least one of the disease states of the disease category when the first confidence score is equal to or greater than a threshold value; or identify the biological sample as not associated with at least one of the disease states of the disease category when the first confidence score is less than the threshold value.

In particular embodiments, the techniques described herein relate to a system, wherein the models are further operable to: identify the biological sample as associated with the respective disease state in the disease category when the second confidence score is equal to or greater than a threshold value; or identify the biological sample as not associated with the respective disease state in the disease category when the second confidence score is less than the threshold value.

In particular embodiments, the techniques described herein relate to a system, wherein the respective disease state is identified with a sensitivity of at least approximately 90%.

In particular embodiments, the techniques described herein relate to a system, wherein the respective disease state is identified with a specificity of at least approximately 94%.

In particular embodiments, the techniques described herein relate to a system, wherein the biological sample is one or more of breath, saliva, urine, stool, skin emanations, tissue, or blood.

In particular embodiments, the techniques described herein relate to a system, wherein the disease category is selected from a group consisting of: cancer, liver disease, gastrointestinal disease, neurological disease, metabolic disease, vascular disease, and infectious disease.

In particular embodiments, the techniques described herein relate to a system, wherein the disease category is cancer, and the one or more disease states is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

In particular embodiments, the techniques described herein relate to a method of disease detection including: receiving a test kit, wherein the test kit includes a biological sample from a patient; exposing the biological sample to a first set of detection animals; accessing a first sensor data associated with the first set detection animals; processing, using a first ML-based disease-detection model trained on a first dataset of detection events, the first sensor data to calculate a first confidence score corresponding to a disease category associated with the biological sample, wherein the disease category includes a plurality of disease states, and wherein the first confidence score indicates a likelihood of at least one of the disease states of the disease category being present in the patient; responsive to the first confidence score being greater than a first threshold score, exposing the biological sample to a second set of detection animals; accessing a second sensor data associated with the second set of detection animals; and processing, using a second ML-based disease-detection model trained on a second dataset of detection events, the second sensor data to calculate one or more second confidence scores corresponding to one or more disease states in the disease category associated with the biological sample, wherein each confidence score indicates a likelihood of a respective disease state in the disease category being present in the patient.

In particular embodiments, the techniques described herein relate to a method, wherein: the first sensor data includes data associated with a conditioned response of the first set of detection animals; and the second sensor data includes data associated with a conditioned response of the second set of detection animals.

In particular embodiments, the techniques described herein relate to a method, further including: receiving a third sensor data associated with the first set of detection animals or the second set of detection animals that have been exposed to one or more of a service sample; and calculating, based on the third sensor data, one or more confidence scores, each corresponding to a positive control category or a negative control category.

In particular embodiments, the techniques described herein relate to a method, wherein the first and second sets of detection animals are exposed to each of the biological sample and the service sample via a sampling port.

In particular embodiments, the techniques described herein relate to a method, further including determining which of a particular sample to expose to the first set of detection animals or the second set of detection animals, wherein the particular sample is selected from a group consisting of: the biological sample from the patient and the service sample.

In particular embodiments, the techniques described herein relate to a method further including: identifying the biological sample as associated with at least one of the disease states of the disease category when the first confidence score is equal to or greater than a threshold value; or identifying the biological sample as not associated with at least one of the disease states of the disease category when the first confidence score is less than the threshold value.

In particular embodiments, the techniques described herein relate to a method, further including: identifying the biological sample as associated with the respective disease state in the disease category when the second confidence score is equal to or greater than a threshold value; or identifying the biological sample as not associated with the respective disease state in the disease category when the second confidence score is less than the threshold value.

In particular embodiments, the techniques described herein relate to a method, wherein the respective disease state is identified with a sensitivity of at least approximately 90%.

In particular embodiments, the techniques described herein relate to a method, wherein the respective disease state is identified with a specificity of at least approximately 94%.

In particular embodiments, the techniques described herein relate to a method, wherein: the first set of detection animals is conditioned to detect the disease category of the biological sample; and the second set of detection animals is conditioned to detect the disease state of the biological sample.

In particular embodiments, the techniques described herein relate to a method, wherein the biological sample is one or more of breath, saliva, urine, stool, skin emanations, tissue, or blood.

In particular embodiments, the techniques described herein relate to a method, wherein the disease category is selected from a group consisting of: cancer, liver disease, gastrointestinal disease, neurological disease, metabolic disease, vascular disease, and infectious disease.

In particular embodiments, the techniques described herein relate to a method, wherein the disease category is cancer, and the one or more disease states is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

In particular embodiments, the techniques described herein relate to a method of disease detection including: receiving a test kit, wherein the test kit includes a biological sample from a patient; exposing the biological sample to a first set of detection animals; accessing a first sensor data associated with each detection animal in the first set of detection animals, processing, using a first ML-based disease-detection model trained on a first dataset of detection events, the first sensor data associated with each detection animal in the first set of detection animals to determine whether the detection animal in the first set of detection animals indicate a disease category to present in the biological sample; in response to a determination that less than a first threshold percentage of the first set of detection animals indicate the disease category to be present in the biological sample, identifying the biological sample as not associated with the disease category; in response to a determination that between the first threshold percentage and a second threshold percentage of the first set of detection animals indicate the disease category to be present in the biological sample, exposing the biological sample to a subset of detection animals from the second set of detection animals; wherein: in response to a determination that less than a threshold fraction of the subset indicated a disease state to be present in the biological sample, identifying the biological sample as not associated with the disease category; and in response to a determination that greater than the threshold fraction of the subset indicated the disease state to be present in the biological sample, identifying the biological sample as requiring exposure to the second set of detection animals; and in response to a determination that greater than the second threshold percentage of the first set of detection animals indicate the disease category to be present in the biological sample, identifying the biological sample as requiring exposure to the second set of detection animals.

In particular embodiments, the techniques described herein relate to a method, further including: exposing the biological sample to a second set of detection animals; accessing a second sensor data associated with each detection animal in the second set of detection animals, processing, using a second ML-based disease-detection model trained on a second dataset of detection events, the second sensor data associated with each detection animal in the second set of detection animals to determine whether the detection animal in the second set of detection animals indicate the disease state to present in the biological sample; in response to a determination that less than a third threshold percentage of the second set of detection animals indicate the disease state to be present in the biological sample, identifying the biological sample as not associated with the disease state; and in response to a determination that greater than the third threshold percentage of the second set of detection animals indicate the disease state to be present in the biological sample, identifying the biological sample as associated with the disease state.

In particular embodiments, the techniques described herein relate to a method for determining a progression of a disease in a patient undergoing a treatment including: accessing patient data indicating the patient previously tested positive for a first disease state in a disease category and has subsequently received treatment for the disease; receiving a new test kit at a time after the patient has received the treatment for the disease, wherein the new test kit includes a new biological sample from the patient; exposing the new biological sample to a set of detection animals; identifying the new biological sample as being associated with a second disease state; comparing the second disease state with the first disease state; and determining the progression of the disease in the patient after the treatment based on the comparing.

In particular embodiments, the techniques described herein relate to a method, further including, accessing a second sensor data associated with the set of detection animals; and processing, using a first ML-based disease-detection model trained on a first dataset of detection events, the second sensor data to calculate a second confidence score corresponding to the second disease state associated with the new biological sample.

In particular embodiments, the techniques described herein relate to a method, wherein the method further includes, prior to accessing the patient data: receiving a prior test kit, wherein the prior test kit includes a prior biological sample from the patient; exposing the prior biological sample to a set of detection animals; accessing a first sensor data associated with the set of detection animals; processing, using a first ML-based disease-detection model trained on a first dataset of detection events, the first sensor data to calculate a first confidence score corresponding to the first disease state associated with the prior biological sample; and identify the biological sample as associated with the first disease state when the first confidence score is equal to or greater than a threshold value.

In particular embodiments, the techniques described herein relate to a method, wherein: the new biological sample is one or more of breath, saliva, urine, stool, skin emanations, tissue, or blood; and the prior biological sample is one or more of breath, saliva, urine, stool, skin emanations, tissue, or blood.

In particular embodiments, the techniques described herein relate to a method, wherein the prior biological sample and the new biological sample are of a same sample type.

In particular embodiments, the techniques described herein relate to a method, wherein the disease category is selected from a group consisting of: cancer, liver disease, gastrointestinal disease, neurological disease, metabolic disease, vascular disease, and infectious disease.

In particular embodiments, the techniques described herein relate to a method, wherein the disease category is cancer, and the disease state is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

In particular embodiments, the techniques described herein relate to a method for training a detection animal to provide a conditioned response to be used with a machine learning-based (ML-based) disease-detection system including steps of: exposing a detection animal to a first biological sample from a subject having a target disease state; training the detection animal to provide the conditioned response by providing the detection animal with a reward for identifying the target disease state; inputting, to the disease-detection system, a first sensor data corresponding to the detection animal, wherein the first sensor data is associated with presence of the target disease state; storing tangibly, in a memory of a computer processor, the first sensor data to obtain a dataset of detection events; and training the ML-based disease-detection system to detect the disease state based on the dataset of detection events.

In particular embodiments, the techniques described herein relate to a method, wherein the conditioned response includes a body pose of the detection animal.

In particular embodiments, the techniques described herein relate to a method, further including repeating each of the steps until a threshold sensitivity is reached by the detection animal.

In particular embodiments, the techniques described herein relate to a method, further including repeating each of the steps until a threshold specificity is reached by the detection animal.

In particular embodiments, the techniques described herein relate to a method, wherein the disease state is from a disease category selected from a group consisting of: cancer, liver disease, gastrointestinal disease, neurological disease, metabolic disease, vascular disease, and infectious disease.

In particular embodiments, the techniques described herein relate to a method, wherein the disease state is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

In particular embodiments, the techniques described herein relate to a method, wherein the first biological sample and second biological sample are one or more of breath, saliva, urine, stool, skin emanations, tissue, or blood.

The present embodiments described herein are directed to a disease-detection system which tracks the behavioral, physiological, and neurological patterns of detection animals in a controlled environment and uses those signals to enhance, verify and increase the accuracy of medical diagnostics. Benefits of the disclosed systems and methods include having high accuracy in high throughput screening and diagnostic laboratory tests resulting in early detection of cancer or cancer remission. For example, the disease-detection system disclosed herein reduces the need for invasive procedures such as biopsies by limiting biopsies to the patients whose biological sample was first identified as positive by the disease-detection system. The system may also improve treatment monitoring by enabling more frequent screenings. The system may also provide cancer survivors with easy, cost-effective, and frequent screenings. Further, the system allows for the screen of large populations to identify positive or high-risk individuals. Additionally, the system ensures high accuracy in interpreting animals' behavior.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a kit, and a system, wherein any feature mentioned in one claim category, e.g., method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 illustrates an example disease-detection method.

FIG. 2 illustrates an example disease-detection method.

FIG. 3 illustrates an example disease-detection method.

FIG. 4 illustrates an example disease-detection method.

FIGS. 5A-5B illustrate an example collection kit.

FIG. 6 illustrates an example collection kit.

FIGS. 7A-7B illustrate an example collection kit.

FIGS. 8A-8D illustrate an example collection kit.

FIG. 9 illustrates an example collection kit.

FIG. 10 illustrates an example collection kit.

FIG. 11 illustrates an example collection kit.

FIGS. 12A-12B illustrates an example collection kit.

FIG. 13 illustrates an example test facility.

FIG. 14 illustrates an example test facility.

FIG. 15 illustrates a second example test facility.

FIG. 16 illustrates an example odor detection system.

FIGS. 17A-17B illustrate an example odor detection system.

FIG. 18 illustrates an example odor detection system.

FIG. 19 illustrates an example odor detection system.

FIG. 20 illustrates an example odor detection system.

FIG. 21 illustrates an example odor detection system.

FIG. 22 illustrates an example odor detection system.

FIG. 23 illustrates an example odor detection system.

FIG. 24 illustrates an example odor detection system.

FIGS. 25A-25B illustrate an example odor detection system.

FIG. 26 illustrates an example odor detection system.

FIG. 27 illustrates an example disease-detection method.

FIG. 28 illustrates an example computing system.

FIG. 29 illustrates an example computing system.

FIG. 30 illustrates an example computing system.

FIG. 31 illustrates a diagram of an example machine-learning (ML) architecture.

FIG. 32 illustrates a diagram of an example machine-learning (ML) architecture.

FIG. 33 illustrates a diagram of an example machine-learning (ML) training method.

FIG. 34 illustrates an example odor detection system.

FIGS. 35A-35D illustrate example disease-detection methods.

FIGS. 36A-36C illustrate example machine-learning (ML) architectures.

FIGS. 37A-37B illustrate an example machine-learning (ML) architecture.

FIG. 38 illustrates an example machine-learning (ML) architecture.

FIG. 39 illustrates example performance characteristics for an example sample round.

FIGS. 40A-40B depict performance data.

FIG. 41 illustrates an example computer system.

FIG. 42 depicts validation data of a disease-detection method.

FIG. 43 depicts experimental results.

FIG. 44 depicts experimental results.

FIG. 45 depicts experimental results.

FIG. 46 depicts experimental results.

FIG. 47 illustrates an example method utilizing brain imaging.

FIG. 48 depicts experimental results utilizing brain imaging.

FIGS. 49A-49B depict performance data.

### DESCRIPTION OF EXAMPLE EMBODIMENTS

Canines have extremely sensitive olfactory receptors and are able to detect specific scent molecules even at low concentrations. This olfactory advantage of canines over humans, coupled with the abilities to train canines to provide a palpable response to a target odor, offers a beneficial screening tool for cancer detection.

The subject matter of the present disclosure is described with reference to the figures. It should be understood that numerous specific details, relationships, and methods are set forth in this Description and accompanying Figures to provide a more complete understanding of the subject matter disclosed herein. For purposes of clarity of disclosure and not by way of limitation, the detailed description is divided into the following subsections:
1. Definitions
2. Disease-Detection Overview
3. User Experience
4. Sample Collection
5. Test facility
6. Olfactometer System
7. Disease-Detection System
8. Machine-Learning Architecture
9. Exemplary Methods
10. Computer System Overview
11. Experimental Results
12. Recitation of Embodiments
13. Miscellaneous

### 1. Definitions

As used herein, the term “sniff” refers to the fundamental testing unit (sampling unit) of the interaction between the detection animal (e.g., dog) and a sample. A sniff may comprise a single sniff, or multiple “sub-sniffs” and either or all of these sniffs would be considered to yield a single response (“sniff result”) from the detection animal.

As used herein, the term “sampling” by the detection animal (e.g., dog) refers to any type of detection, including, but not limited to sniffing (smell, olfactory detection), visual detection, detection by taste, or detection by touch.

As used herein, the term “sampling port” refers to an interface through which the detection animal (e.g., dog) encounters a sample by any means including, but not limited to smell, vision, taste, and touch. For example, a sampling port through which the detection animal encounters the smell of a sample may also be referred to a “sniffing port.”

As used herein, the term “disease category” refers to the various families of disease, such as, but not limited to cancer, neurodegenerative disease, vascular disease, and infectious disease. As used herein, the term “disease state” refers to a specific type of disease with a family. As an example, and not by way of limitation, the disease category of “cancer” may include the disease states of breast cancer and lung cancer, and the disease category of “neurodegenerative disease” may include the disease states of Alzheimer's disease and Parkinson's disease.

As used herein, the term “sampling round” may refer to steps performed to detect the presence of a disease category (e.g., cancer, neurodegenerative disease) through a first stage of testing (“horizontal test”), or presence of a specific disease state (e.g., breast cancer, lung cancer, prostate cancer, brain cancer, ovarian cancer, skin cancer, or colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer) through a second stage of testing (“vertical test”). In general, in the sampling rounds, the detection animals (e.g., dogs) are exposed to one or more samples (e.g., breath sample), each of which would be sampled (e.g., sniffed) to yield a response input from the detection animals.

As used herein, the term “test sample” may refer to a biological sample collected from a patient for disease detection.

As used herein, the term “service sample” may refer to a biological sample with a disease category or disease state that is known. Example types of “service samples” include known positive samples and known negative samples. In particular embodiments, the purpose of the service sample is to assess the accuracy of the disease detection model.

### 2. Disease-Detection System Overview

In particular embodiments, a disease-detection system for detection animals for medical diagnostics may comprise a combination of sensors, cameras, operational systems, and machine learning (ML) algorithms, which may serve one or more of the following purposes: (1) real-time management of the screening tests in the lab, which include presenting the test's setting and events in real-time on the lab manager's monitor or guiding the lab manager on how to operate the test based on the test protocol, (2) management of the testing facility's resources and clients, including patients, samples, canines, handlers, and lab managers, (3) management of monitoring and analytics which support training plans of detection animals, (4) management of communications with the customer, the customer's healthcare provider(s), third parties, and the screening centers, including customer subscriptions, sample shipping, payment, and laboratory results communication, in both direct-to-consumer and business-to-business-to-consumer scenarios, (5) collecting and synchronizing data from different sources and providing raw data to the testing facility, (6) providing test data in real-time to the disease-detection system, (7) providing analytical-based recommendations for lab results, including a positive/negative test result and a confidence score, at difference stages of the testing process, (8) a real-time monitoring and alerting system, which ensures the quality of the testing facility's product and resources, as well as alerts whenever an anomaly is detected, (9) identification of the type of cancer present or the cancer stage in a biological sample by analyzing the detection animal's brain imaging (e.g. from an EEG, fNIR, fMRI, or MRI), or (10) using the detection animal's brain imaging as a verification step.

The system may track and monitor hundreds of signals at every second produced in real time by detection animals (e.g., cancer-sniffing dogs) as the detection animals are exposed to the samples in the laboratory and combine the signals with medical data. The result may be an accurate, non-invasive, and fast screening test for one or more disease states (e.g., cancer), with a higher level of sensitivity than devices or screening tests which are used in medicine today.

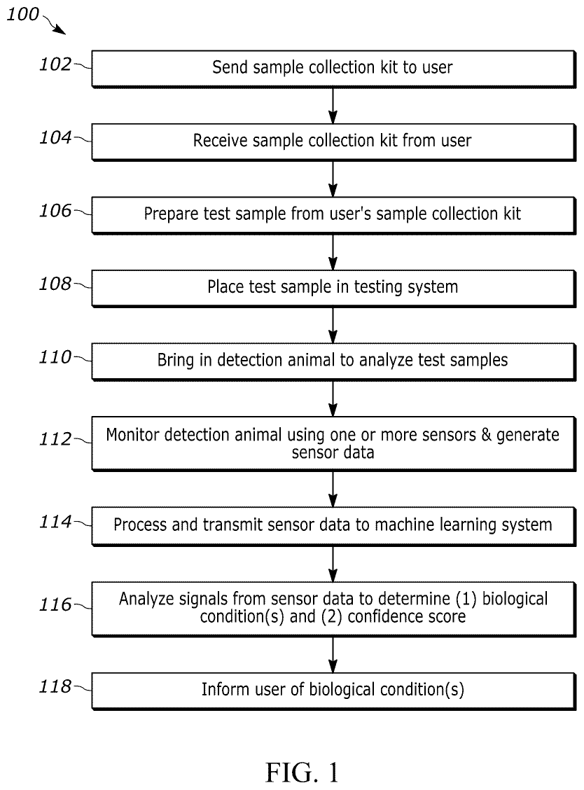

FIG. 1 illustrates a flow diagram of a method 100 for a disease-detection system in accordance with the presently disclosed embodiments. The method 100 may be performed utilizing one or more processing devices that may include hardware, e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), or any other processing device(s) that may be suitable for processing sensor data, software (e.g., instructions running/executing on one or more processors), firmware (e.g., microcode), or some combination thereof. In particular embodiments, the disease-detection system comprises one or more ML-models (e.g., a ML-based disease-detection model). The disease-detection system may further comprise one or more additional computing components, including a monitoring component and an operational component.

The method 100 may begin at step 102 with the testing facility, either directly or through an affiliate, sending a sample collection kit to a user after receiving a request from a user (e.g., a patient) or the user's physician. In particular embodiments, a customer ID is assigned to the user and the customer ID is associated with the user's biological sample through the life cycle of the biological sample. In particular embodiments, a physician may order a general screening test. In other embodiments, a physician may order a diagnostic test for one or more diseases in response to the user communicating the presence of particular symptoms. In particular embodiments, the sample collection kit comprises a collection device and user instructions. In particular embodiments, the collection device may be a facial mask or a surgical mask that the user breathes into for a specified amount of time. In particular but non-limiting embodiments, the collection device may be a tube, a cup, a bag, or any suitable collection kit which may be used to collect a biological sample. As an example, and not by way of limitation, in a particular embodiment, the user receives a collection device and is instructed to breathe into the collection device for three minutes. As another example, and not by way of limitation, in a particular embodiment, the user receives a collection device and is instructed to breathe into the collection device for five minutes. As an example, and not by way of limitation, the sample collection may be performed from home, at a survey institute, at a clinic, or any other location suitable for sample collection. The full life cycle of the sample, from activation to extermination, is tracked with a high level of traceability.

The method 100 may then continue at step 104 with the test facility receiving the sample collection kit from the user. Upon receipt of the sample collection kit, the test facility processes the kit by labeling the sample with an identification number corresponding to the user and enters information related to the received sample into the disease-detection system. In particular embodiments, the disease-detection system may contain information about the user, such as name, age, prior health history, family health history, lifestyle habits, etc.

The method 100 may then continue at step 106 with a person or a machine preparing a biological sample from the user's sample collection kit. In particular embodiments, a person or a machine performs a method of extracting chemical molecules out of the biological sample. In particular embodiments, a lab worker may open the collection device, e.g. a mask, and split the mask into two or more parts so that there is at least a biological sample (test sample) and a backup sample. In particular embodiments, one of the parts of the biological sample may be used for testing by traditional methods, such as by gas chromatography mass spectrometry (GCMS) or biopsy. In particular embodiments, the lab worker may put the biological sample into a receptacle operable to be attached to an olfactometer. In particular embodiments, the lab worker may put the biological sample into a container for introduction into the testing room. In a particular but non-limiting example, the container is a glass container with one or more small openings which allow for a detection animal to detect the scent inside the container. In particular embodiments, preparing the biological sample may be automated using robotics and other machines. In particular embodiments, preparing the biological sample comprises attaching a container containing the biological sample to an olfactometer system. In particular embodiments, the method of receiving the biological sample and preparing the biological sample occurs in a sterile environment.

The method 100 may then continue at step 108 with a person or machine placing the biological sample into the testing system. In one embodiment, the testing system is an olfactometer system, wherein the samples are placed into a receptacle of an olfactometer system, wherein the olfactometer system comprises a plurality of receptacles, and wherein each receptacle is connected to a sampling port. In one embodiment, the receptacles and the sampling port are connected, but the receptacles and the sampling port are in separate rooms. The structure of the olfactometer system is discussed herein. The structure of an example testing room and testing facility is discussed herein. The testing room contains a plurality of sampling ports. In one embodiment, a biological sample is placed in a receptacle of the sampling port. In a particular embodiment, the sampling ports are connected to an olfactometer system. In a particular embodiment, the sampling port is connected to a receptacle, which is operable to hold a biological sample. The testing room is configured to hold the biological samples of a plurality of users. In particular embodiments, each receptacle contains the biological sample of a different user.

In particular embodiments, a breath sample is validated as a biological sample from a patient. In particular embodiments, this step is performed prior to exposing the biological sample to the detection animal. As an example, and not by way of limitation, one or more breath sensors confirms the biological sample is a breath sample from the patient. As an example, and not by way of limitation, the breath sensor may be one or more of a TVOC sensor, breath VOC sensor, relative humidity sensors, temperature sensor, photoionization detector (PID), flame ionization detector (FID), or metal oxide (MOX) sensor. As an example, and not by way of limitation, the one or more breath sensors is located upstream of the sampling port.

The method 100 may then continue at step 110 with a person or a machine bringing in one or more detection animals to analyze the biological samples in the testing room. In particular embodiments, a detection animal enters the testing room to sniff each sampling port. The animal may enter with a handler (e.g., to guide the animal to the biological samples) or without a handler. In particular embodiments, the detection animal walks around the testing room (with or without a handler) to sniff each sampling port to detect one or more target odors. In particular embodiments, the detection animal goes to each sampling port and sniffs each sampling port to detect one or more target odors. In particular embodiments, the detection animal performs a run, wherein a run comprises sniffing each sampling port in the testing room. In particular embodiments, the detection animal performs several runs. In particular embodiments, biological samples are transferred to a different sampling port in the testing room in between runs and the detection animal is brought in after the samples are transferred to perform another run.

In particular embodiments, if the detection animal provides the same results in two different runs, then the system determines the result to be valid, and instructs a person or machine to bring a second detection animal to the testing room to perform a run. In particular embodiments, if the detection animal provides different results in two or more runs, the detection animal repeats the process of sniffing each sampling port until a consistent result is established, or until the detection animal has reached a maximum number of allowed runs per session. Although this disclosure describes a particular protocol for a run, this disclosure contemplates any suitable protocol for a run. The detection animal may be any suitable non-human animal with olfactory senses, such as a canine. Although this disclosure describes analyzing biological samples with particular types of detection animals, this disclosure contemplated analyzing biological samples with any suitable type of detection animal. As an example, and not by way of limitation, other suitable types of detection animals may include grasshoppers, ants, bears, and rodents, such as rats and mice. In particular embodiments, upon the positive identification of a target odor, the detection animal may be provided with a reward by either a human or a machine executing an automated reward mechanism. In particular embodiments, the reward may be one or more of: a food, a toy, or positive feedback from a human or machine. In particular embodiments, an additional detection animal is brought into the testing room to sniff the sampling port to detect a particular target odor. In particular embodiments, one or more different detection animals are brought into the testing room, one after the other, to detect for target odor(s) in each sampling port. In particular embodiments, five detection animals may be used to analyze a particular set of samples in the testing room. In particular embodiments, the decision of whether a particular sampling port contains a target odor is made by analyzing signals generated from all canines in a particular test session. In particular embodiments, the process of operating and monitoring the test procedure may be automated.

In particular embodiments, a canine may indicate a particular sample to contain the target odor by performing a trained (conditioned) action. In particular embodiments, the trained action may comprise a body pose. A body pose may include, but is not limited to, standing next to the sampling port, sitting next to the sampling port, looking at a handler, or lying next to the sampling port. In particular embodiments, the trained action may comprise an act, such as emitting a sound. In particular embodiments, after a detection animal indicates a particular sample to contain the target odor, that particular sample is removed from the testing room and the detection animal performs one or more additional runs to detect target odors in the remaining samples. Although this disclosure describes detection animals performing a trained action in a particular manner upon detection of a target odor, this disclosure contemplates any suitable trained action upon detection of a target odor.

In particular embodiments, detection animals are selected based on one or more of their natural abilities which include: odor detection abilities, strength, natural instincts, desire to please humans, motivation to perform certain actions, sharpness, tendency to be distracted, or stamina. In particular embodiments, detection animals are trained through operant conditioning, which encompasses associating positive behavior with a reward, negative behavior with a punishment, or a combination thereof. In particular embodiments, detection animals are trained using only a reward-based system. In particular embodiments, detection animals are taught to sit when they detect a target odor. In particular embodiments, detection animals may be taught to identify a plurality of target odors and exhibit a particular behavioral, physiological, or neurological response upon identification of a particular target odor. As an example, and not by way of limitation, the target odor is a cancer VOC profile. In particular embodiments, a trainer may teach a detection animal to associate a target scent with a reward. In particular embodiments, an animal may be trained on odors through a sample which contains a mixture of various odors. In particular embodiments, a trainer may present odors separately but train animals on odors at the same time (intermixed training). In particular embodiments, the detection animal may be trained to exhibit a different response for different stages of cancers or different types of cancers. Although this disclosure describes training detection animals in a particular manner, this disclosure contemplates training detection animals in any suitable manner.

In particular embodiments, detection animals undergo a multi-level training program. As an example, and not by way of limitation, detection animals may undergo a three-level training program which may comprise a first-level training program for preparing the detection animal, a second-level training program for developing abilities of outcomes detection, and a third-level training program for developing assimilation of sniffing abilities and simulation of real situations. In particular embodiments, the first-level training program comprises one or more of: leash training, basic discipline training, socialization (e.g. exposure to external stimulations during work wherein the stimulation includes one or more of other animals, cars, or people), or training basic scanning technique. In particular embodiments, the second-level training program comprises one or more of: assimilation of the outcome scent (e.g. combining food into the training), assimilation of the outcome scent (e.g. weaning from food combination), advanced discipline training, exposure to various distractions and outcomes, or increasing scan volumes and sniffing quality. In particular embodiments, the third-level training program comprises one or more of: assimilation of various cancer scents, combination(s) of different scents for detection, assimilation of various outcome scents and concentrations, combination(s) of different scents for detection, exposure to complex outcomes, or simulations of real-life situations. The training may be done in a double-blind manner, such that neither the handler nor persons handling training samples know whether test samples are positive or not during the training. In particular embodiments, the detection animals may pass a first level of training before moving onto the next level of training. Although this disclosure describes training detection animals in a particular manner, this disclosure contemplates training detection animals in any suitable manner.

In particular embodiments, the detection animal may be trained to detect a particular type of cancer, while ignoring other types of cancer. For example, a detection animal may be trained to detect a biological sample from a patient having breast cancer, while ignoring a biological sample from a patient having colon cancer or lung cancer. In particular embodiments, the detection animal may be trained to detect a particular type of cancer by exposing multiple types of cancer during training, but rewarding the detection animal only when it detects the particular type of cancer.

In particular embodiments, detection animals are not trained (e.g., unconditioned animals) to exhibit specific behavioral responses in response to specific biological samples. In particular embodiments, a detection animal (e.g., a canine) may have specific responses (e.g., a neurological response or a behavioral response) to particular odors. As an example,

In particular embodiments, a combination of conditioned and unconditioned responses may be used. Non-limiting examples of conditioned or unconditioned responses include monitoring of sitting (or not sitting), changes in face gestures, change in body position, and neurological features of the detection animal.

The method 100 may then continue at step 112 with one or more of sensors collecting data in real-time from the testing room and from the detection animal. In particular embodiments, the one or more sensors comprise one or more behavioral sensors, one or more physiological sensors, one or more neurological sensors, one or environmental sensors, and one or more operational sensors.

As an example, and not by way of limitation, behavioral sensors may comprise one or more of: cameras, audio recorders, accelerometers, thermal sensors, or distance sensors which monitor the behavior of the detection animals as the animals detect for scents in the sampling ports. In particular embodiments, video-recorders and/or cameras may transmit images of the detection animals and data containing timestamps of the images, which may enable calculations including a duration of a sniff. A duration of a sniff is the time the detection animal spends sniffing a particular sample. In particular embodiments, the cameras may transmit frames from a plurality of angles, and the frames are analyzed to extract measurements such as a duration of a sniff, a number of repeated sniffs, a time a detection animal spent at a sampling port, a body pose of the animal (e.g., whether the animal is sitting, or whether the detection animal looks at its handler), facial features or facial gestures of the animal, a body position of the animal (e.g., skeletal position), or neurological features of the detection animal. In particular embodiments, image data (e.g., from a camera/video record) may comprise a sitting detection outcome (e.g., an indication of whether a detection animal sits down after being exposed to a biological sample). Using the sitting detection outcome, the disease-detection system can also measure the sitting duration and a time between sniffing to sitting, which may be input into a ML-model. In particular embodiments, the disease-detection system calculates the amount of time between a sniff and the moment the animal signals it found a target odor. In particular embodiments, audio sensors transmit the sounds of the sniffs, which may include the duration and intensity of a particular sniff.

In a particular embodiment, a behavioral sensor may be worn by a detection animal. As an example, and not by way of limitation, a behavioral sensor may comprise one or more of: accelerometer, a gyroscope, or a camera. In particular embodiments, the behavioral sensor provides information about the animal's movements and behavior in the testing room. In particular embodiments, a distance sensor (e.g., an ultrasonic sensor, an infrared sensor, a LIDAR sensor, or a time-of-flight distance sensor) may detect the behavior of an animal, including when the duration that the detection animal's head is in or near the sampling port. Although this disclosure describes sensors in a particular manner, this disclosure contemplates any suitable sensors measuring any suitable measurements.

As an example, and not by way of limitation, physiological sensors may comprise one or more of a: heart rate monitor, heart rate variability monitor, temperature sensor, galvanic skin response (GSR) sensor, or a breath rate sensor. In particular embodiments, the physiological sensor may be worn by the detection animal. In particular embodiments, the physiological sensor is not worn by the detection animal. Although this disclosure describes sensors in a particular manner, this disclosure contemplates any suitable sensors measuring any suitable measurements.

As an example, and not by way of limitation, neurological sensors may comprise one or more of sensors operable to gather: Electroencephalogram (EEG), Functional Near Infrared Spectroscopy (fNIR), Magnetic Resonance Imaging (MRI), or Functional Magnetic Resonance Imaging (fMRI). As an example, and not by way of limitation, the sensor may comprise an EEG cap worn on the head of a detection animal to monitor the animal's neurological signals. Although this disclosure describes sensors in a particular manner, this disclosure contemplates any suitable sensors measuring any suitable measurements.

As an example, and not by way of limitation, environmental sensors may comprise one or more of: temperature sensors, humidity sensors, noise sensors, or air sensors. In particular embodiments, environmental sensors may measure air particulate levels or air filtration levels, including air pollution levels and the rate of air exchange in the testing room. In particular embodiments, environmental sensors may include noise sensors which measure the noise level of the testing room. In particular embodiments, environmental sensors may comprise one or more gas sensors, including a chemical or electrical sensor that can measure a total amount of VOCs or detect the presence of a particular VOC. In particular embodiments, the gas sensor can detect a quality or quantity of an inorganic gas (such as one or more of CO₂, CO, N₂, or O₂), wherein the inorganic gas is correlated to a quality or quantity of a biological sample. In particular embodiments, sensors are placed at receptacles which contain biological samples to collect measurements at the receptacles. Example sensors include: a gas sensor to measure a VOC quality or quantity, an audio sensor to measure one or more auditory features (e.g., a sound, duration, or intensity of a sniff), an infrared sensor to measure a duration of a sniff, or a pressure sensor to measure a pressure of the detection animal's nose against a sampling port. Although this disclosure describes sensors in a particular manner, this disclosure contemplates any suitable sensors measuring any suitable measurements.

As an example, and not by way of limitation, operational sensors may comprise one or more of: sensors in an olfactometer system, sensors for animal management (e.g., a RFID card which identifies a particular canine), and sensors for sample management (e.g., a QR code scanner which scans a unique QR code associated with each biological sample). In particular embodiments, step 112 comprises real-time monitoring and analysis, described herein. In particular embodiments, Step 112 comprises managing operational data received from the operational sensors described herein, including data corresponding to sensor performance, sample tracking, and detection animal tracking. Although this disclosure describes sensors in a particular manner, this disclosure contemplates any suitable sensors measuring any suitable measurements.

The method 100 may then continue at step 114 with processing and transmitting certain data obtained from the various sensors to one or more ML-models. In particular embodiments, the disease-detection system collects data from a plurality of sensors comprising one or more of behavioral, physiological, and neurological sensors. In particular embodiments, the sensors measure one or more of: animal behavior, animal physiological patterns, or animal neurological patterns. In particular embodiments, processing data comprises synchronizing data, ensuring data security, transforming raw data into refined data which is input into one or more ML-models, managing laboratory resources, and performing test and training analytics.

At step 116, one or more ML-models analyzes one or more signals from the sensor data to determine one or more biological conditions and a confidence score. As an example, and not by way of limitation, the one or more ML-models comprise one or more of: one or more ML-models for a particular detection animal (e.g., a dog-specific ML-model), one or more ML-model for a plurality of detection animals (e.g., a dog pack-specific ML-model, also referred to herein as a “lab-result ML-model”), one or more test stage-specific models (e.g., a ML-model for stage 1 of a test), one or more ML-models trained on disease states (e.g. a positive or negative determination of cancer), one or more ML-models trained on cancer types (e.g., breast cancer, lung cancer, colon cancer, prostate cancer), one or more ML-models trained on cancer stages (e.g., stage 1, stage 2, stage 3, or stage 4), one or more neurological-based ML-models, or one or more monitoring ML-models (e.g., monitoring the behavioral drift of a detection animal). In particular embodiments, an ML-model may be configured to detect a particular stage or type of cancer (e.g., cancer at stage 2, a breast cancer at stage 2, a breast cancer, etc.). In particular embodiments, the ML-model is operable to perform a monitoring or a predictive function. Although this disclosure describes ML-models in a particular manner, this disclosure contemplates any suitable ML-model for disease detection.

In particular embodiments, the confidence score is calculated based on a probability of the disease state. In particular embodiments, the confidence score is calculated based on a probability of the disease state and a confidence prediction interval. In particular embodiments, the one or more ML-models predict a disease state and likelihood value(s) of the disease state(s) by amplifying and analyzing one or more of: animal behavior (such as a duration of a sniff, a body pose, etc.), physiological patterns, and neurological signals, or inputted patient data. Inputted patient data includes one or more of: family medical history, patient medical history (including lifestyle), patient age, patient gender, or patient demographical data. As an example, and not by way of limitation, patient medical history may include whether or not the patient smokes, the patient's alcohol consumption, and other lifestyle factors.

In particular embodiments, the ML-based disease-detection model is trained on a dataset of target odors and detection events. As an example, and not by way of limitation, detection events may include one or more of signals relating to: animal behavior, physiological signals, or neurological signals. In particular embodiments, the biological condition may be one or more of: a cancer (e.g., breast cancer, lung cancer, prostate cancer, or colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer), Helicobacter pylori (H. pylori) infection, inflammatory bowel disease, or Crohn's disease. In particular embodiments, the biological condition may also include a particular stage of cancer or a particular type of cancer.

The method 100 may then continue at step 118 with the disease-detection system informing the user or the user's doctor of one or more biological conditions and a confidence score associated with each condition.

Particular embodiments may repeat one or more steps of the method of FIG. 1, where appropriate. Although this disclosure describes and illustrates particular steps of the method of FIG. 1 as occurring in a particular order, this disclosure contemplates any suitable steps of the method of FIG. 1 occurring in any suitable order. Moreover, although this disclosure describes and illustrates an example method for ML-based disease-detection of behavioral, physiological and neurological patterns of detection animals including the particular steps of the method of FIG. 1, this disclosure contemplates any suitable method for ML-based disease-detection by monitoring and analyzing behavioral, physiological and neurological patterns of detection animals including any suitable steps, which may include all, some, or none of the steps of the method of FIG. 1, where appropriate. Furthermore, the ML algorithms and functions of the ML-models described herein may include deep learning algorithms, supervised learning algorithms, or unsupervised learning algorithms.

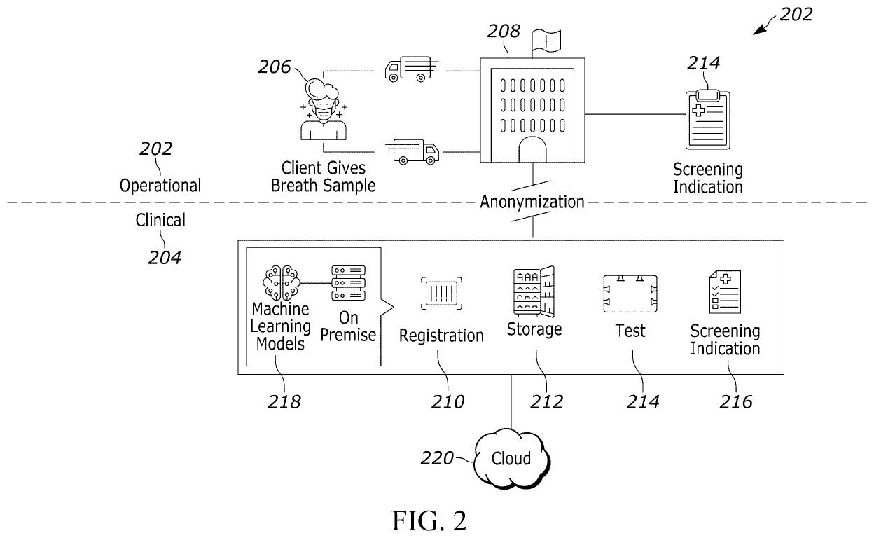

FIG. 2 depicts a disease-detection system which comprises an operational component 202 and a clinical component 204. The operational and clinical components are strictly separated, and all medical records stored on the system are anonymized, encrypted, and do not allow for client identification.

The operational component 202 handles the patient-facing workflow, including the logistics, activation, and authentication of sample kits, test instruction and guidance, and sample management. For example, the operational component comprises obtaining a breath sample from a client 206. In particular embodiments, the breath sample is collected by a medical professional, who then documents the sample collection into a database. The database, which further comprises medical information of the patient, is sent to the clinical facility. Further, the breath sample is sent to a clinical or test facility 208 for testing. The operational component provides a wide range of filtering and sorting capabilities which allow the lab team to retrieve and monitor each and every sample.

The clinical component 204 handles the clinical workflow including: sample registration 210 and management, sample storage 212, sample testing 214, and providing a screening indication 216. Upon arrival of the sample, the sample is recorded and stored. In particular embodiments, samples may be stored at room temperature for up to one year. Although this disclosure describes storing samples in a particular manner, this disclosure contemplates storing samples in any suitable type of manner.

The testing is performed using ML-models 218, which receives data from behavioral sensors, environmental sensors, physiological sensors, neurological sensors, as well as patient data. The clinical component 204 aggregates data in a robust database and supports complex and flexible reporting systems. The data is streamed and processed, and different databases comprising raw data, target odors, and detection events are stored locally in the lab's server, as well as on the cloud 220.

Moreover, although this disclosure describes and illustrates an example method for a disease-detection system including the particular system of FIG. 2, this disclosure contemplates any suitable method for a disease-detection system including any suitable steps, which may include all, some, or none of the system components of FIG. 2.

### 3. User Experience

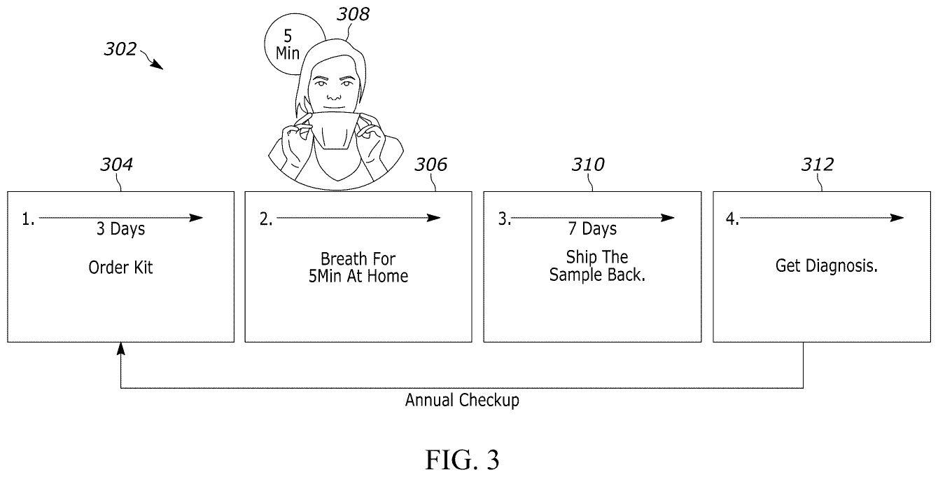

FIG. 3 illustrates a flow diagram of an example method of screening and diagnostics from the user perspective. The method 302 may begin at step 304 with a user (e.g., a patient) or a physician ordering a test. In particular embodiments, a high-risk patient (e.g., one that is at a high risk for breast cancer) may be identified by a physician or by a database comprising patient data. In particular embodiments, a patient may be identified as high-risk after completing a questionnaire about their family medical history and personal medical history.

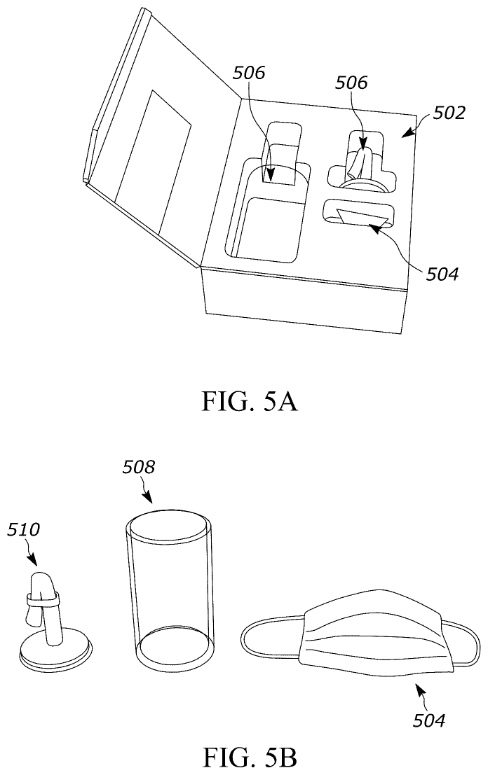

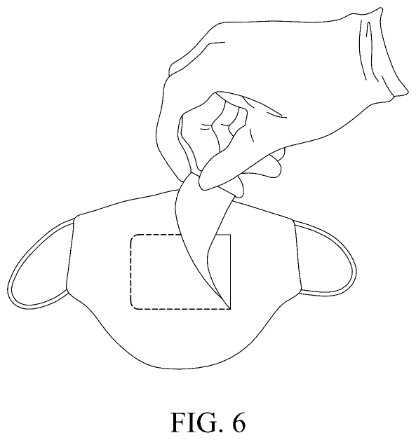

In particular embodiments, the user receives a sample collection kit which contains a collection device. The sample collection kit is discussed herein. In a particular embodiment but non-limiting embodiment, the collection device is a facial mask which the user may breathe into. Next, at step 306, the user 308 breathes into the facial mask. In an example embodiment, the user 308 breathes into the facial mask for five minutes. In particular embodiments, the user may perform some other biological function to enable the user's biological sample to be placed into the collection device. For example, the user may swab their mouth and place the swab into a collection device. As another example, the user may collect their urine in a collection device. Next, at step 310, the user packs the biological sample into shipment packaging and ships the sample to the test facility. Next, at step 312, the user receives the results, which may include a diagnosis. In particular embodiments, the diagnosis includes an identification of one or more biological conditions and a confidence score of each biological condition. FIGS. 5A, 5B and 6 depict non-limiting examples of a sample collection device. For example, the collection device may be a tube, a cup, or a bag, or any suitable collection kit which may be used to collect a biological sample. In particular embodiments, the biological sample may be one or more of: breath, saliva, urine, feces, skin emanations, stool, biopsy, or blood. Although this disclosure describes biological samples in a particular manner, this disclosure contemplates biological samples in any suitable manner.

### 4. Sample Collection

### 4.1. Sample Collection Protocol

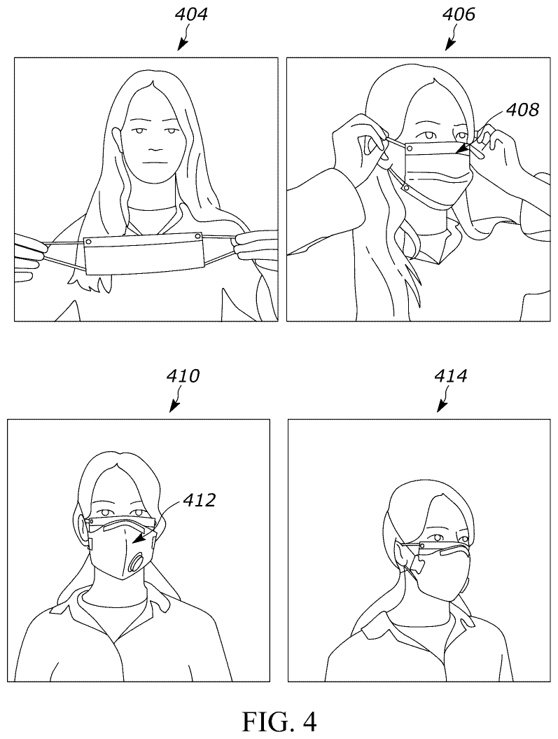

FIG. 4 depicts an example sample collection protocol. Samples may be collected at a patient's home or in a medical facility. An example collection protocol 402 is described below. Although this disclosure describes an example protocol for obtaining a biological sample, this disclosure contemplates any suitable method for obtaining a biological sample.

Patients are instructed to not smoke for at least two hours before breath collection. Patients are instructed to not consume coffee, alcohol, or food for at least an hour before breath collection. The patient is instructed to breathe only through the mouth, and not through the nose. First, at step 404, the patient performs a “lung wash” step wherein the patient breathes in a normal, relaxed manner for one minute. Next, the patient is instructed to take a full breath so that the full volume of the lungs is filled, and then to hold the breath for at least five seconds. Then, at step 406, the patient puts on a first mask 408 (e.g. the “sample collection mask”). Next, at step 410, the patient puts on a second mask 412 (e.g. the “isolation mask”) over the first mask. The purpose of the second mask is to filter the incoming air from the environment that the patient inhales. In particular embodiments, the second mask may be placed over the first mask such that a predetermined gap is formed between the first mask and the second mask. The purpose of this space between the first mask and the second mask is to increase the VOC absorbance by the first mask. For instance, the first mask (e.g. the sample collection mask) has a first portion which faces the patient and a second portion which faces away from the patient. In particular embodiments, the first mask may fit snugly against a patient's mouth and nose. As a person exhales, the exhaled air is first passed through the first portion of the first mask, and the first portion collects the breath and aerosols exhaled by a patient. Then, the second portion of the first mask, which is in the predetermined gap formed between the first mask and the second mask, is operable to passively absorb the breath and aerosols exhaled by the patient.

The sample collection mask and the isolation mask are described in further detail herein. In a particular embodiment, the patient holds their breath for a minute, the protocol continues at step 414, wherein the patient should breathe normally, only through their mouth, for at least three minutes. A benefit of this example of this example breathing and collection protocol is to maximize the collection of alveolar breath from the patient. Alveolar breath is breath from the deepest part of the lung.

The first mask and the second mask should cover the patient's nose and mouth. Further, there may be minimal gaps between the mask and the patient's face, to allow for all inhaled and exhaled air to go through the mask. Additionally, patients should not talk during the sample collection procedure while they are wearing the sample collection component. After the patient has breathed through their mouth for five minutes, while wearing both the first mask and the second mask, the second mask is carefully removed. Then, the first mask is removed. In particular embodiments, the first mask is removed using sterile gloves, and the mask is folded in half by touching only the outer layer of the mask. Next, the mask is inserted into a storage component, e.g. a bag or a container, sealed, and then transported to a test facility. In particular embodiments, the second mask (e.g. the isolation mask) is discarded.

Although this disclosure describes and illustrates an example method for sample collection including the particular steps of the method of FIG. 4, this disclosure contemplates any suitable method for sample collection including any suitable steps, which may include all, some, or none of the steps of the method of FIG. 4.

In particular embodiments, a patient's breath sample may be collected at the patient's home, or at a place other than a medical office. In particular embodiments, the patient is instructed to collect their sample at an instance when they have not eaten or drank alcohol within two hours prior to sample collection, not smoked within two hours prior to sample collection, and are not feeling ill during the sample collection. In particular embodiments, the patient may register the test with a medical provider by scanning a QR code associated with the sample collection kit. In particular embodiments, the sample collection kit includes a set of gloves, and the patient is instructed to wear the gloves (e.g., surgical gloves). Further, after wearing the gloves, the patient is instructed to not touch anything but the components of the sample collection kit. In particular embodiments, the patient is instructed to take three deep breaths, and then put on a first mask. In particular embodiments, the first mask comprises the sample collection component. Optionally, the patient is then instructed to put on a second mask. In particular embodiments, the patient is instructed to breathe normally for no less than three minutes. In particular embodiments, the patient is instructed to open the storage component. In particular embodiments, the patient is instructed to open the outer lid of the storage component by turning the outer lid anti-clockwise until it opens, which causes the outer lid to open. In particular embodiments, if the outer lid does not open, the patient may also pull the outer lid. In particular embodiments, after the outer lid is open, the patient is instructed to pull on a part of the inner lid to open said inner lid. In particular embodiments, after the patient has breathed for at least three minutes while wearing the mask, the patient is instructed to remove a cartridge of the mask. In particular embodiments, the cartridge is a detachable part of the mask. In particular embodiments, the patient is instructed to place the cartridge of the mask in the storage component, and close the inner lid. In particular embodiments, the patient is instructed to close the outer lid by turning it clock-wise, which causes the storage component to be locked. In particular embodiments, the patient is instructed to send the sample collection kit containing the collected patient sample to a laboratory for testing. Although this disclosure describes and illustrates an example method for sample collection, this disclosure contemplates any suitable method for sample collection including any suitable steps, which may include all, some, or none of the steps of the method described herein.

### 4.2. Sample Collection Kit

In particular embodiments, the sample collection kit contains a collection device which collects a biological sample that could be one or more of breath, saliva, sweat, urine, other suitable types of samples, or any combination thereof. The samples may contain VOCs or aerosols, which may be detectable by a detection animal. Starting an early stage in the development of cancerous tumors, VOCs are released from the cells to their microenvironment and to the circulation system. From the circulation system, VOCs can be further secreted through other bio-fluids such as through aerosols, gases, and liquid droplets from the respiratory system. Each type and stage of cancer has a unique odor signature created from the either different or the same VOCs in different combinations and proportions. By breathing into the collection kit over several minutes, VOC biomarkers originating from all around the body may be captured with high sensitivity. Although this disclosure describes biological samples in a particular manner, this disclosure contemplates biological samples in any suitable manner.

FIGS. 5A-5B illustrate an example sample collection kit. FIG. 5A depicts a sample collection kit comprising a box 502 which houses a sample collection component 504 (e.g., a mask) and a storage component 506. FIG. 5B depicts an example sample collection component 504 and storage component 506 removed from the box.

### 4.3. Sample Collection Component

The sample collection component is operable to absorb aerosols and droplets which contain VOCs into the sample collection component. Further, the sample collection component is operable to adsorb gaseous molecules (e.g., VOCs) onto the surface of the sample collection component. In particular embodiments, the sample collection component is formed of a plurality of layers, wherein each layer is made of polypropylene. For example, the sample collection component may be an off-the-shelf 5-layer polypropylene mask. For instance, the off-the-shelf mask may be an N-95 or a KN-95 mask. In particular embodiments, the polypropylene absorbs aerosols and liquid droplets from the patient. In particular embodiments, the sample collection component has a filtering efficiency of 95% for particles of 0.3 micron or more. In particular embodiments, the sample collection component may also comprise an active carbon layer which is operable to adsorb VOCs. In other embodiments, the sample collection component comprises two layers of polypropylene and one layer of active carbon. Based on the above descriptions, including at least the desired filtration level and the desired absorptive and adsorptive properties, this disclosure contemplates using any materials which may be suitable to achieve the desired function of the sample collection component.

FIG. 6 depicts an example sample collection component. In particular embodiments, the sample collection component comprises a cartridge which may be detached from the mask. In particular embodiments, the cartridge is made of the same materials as the mask. In particular embodiments, after a patient breathes for three minutes while wearing a mask comprising the cartridge, the patient may remove the cartridge from the mask and place the cartridge in the isolation component.

### 4.4. Isolation Component

The isolation component is operable to provide a barrier between the environment and the sample collection component, to enable the patient to inhale clean air. For example, the isolation component protects the sample collection layer from contamination by the external environment; the contamination may be from ambient pollution or external VOCs/aerosols from someone other than the patient. In particular embodiments, the isolation component is made of polypropylene. In other embodiments, the isolation component may be formed of cotton. In particular embodiments, the isolation component further comprises an active carbon layer for improved filtering. In particular embodiments, the isolation component is rigid such that when the patient wears the isolation component over the sample collection component, there is a gap between the sample collection component and the isolation component. In particular embodiments, this gap maintains space for breath to accumulate in the gap such that additional VOCs may be collected by the sample collection component. For example, the gap increases the amount of gaseous VOCs adsorbed on the outer surface of the sample collection component. In particular embodiments, the isolation component creates a greater volume over the patient's mouth and nose than the sample collection component.

In particular embodiments, the sample collection component and the isolation component are combined into one device.

Based on the above descriptions, including at least the desired filtration level and the desired rigidity or space-maintaining capabilities, this disclosure contemplates any other materials which may be suitable to achieve the desired function of the isolation component.

### 4.5. Storage Component

The storage component is operable to maintain a barrier between the collected biological sample and the external environment and maintains sterility through at least the receipt of the biological sample by the testing facility. In particular embodiments, the storage component prevents the biological sample (e.g., the exhalant) from being exposed to environmental contamination during transport. In particular embodiments, the storage component prevents the biological sample from leaking or from being diluted. In particular embodiments, the storage component is resealable. In particular embodiments, the storage component is heat resistant. In particular embodiments, the storage component has a minimal scent signature.

FIG. 5B depicts an example storage component 506 and a sample collection component 504. In particular embodiments, the storage component 506 may comprise a receptacle 508 and a cap 510, wherein the cap further comprises a seal.

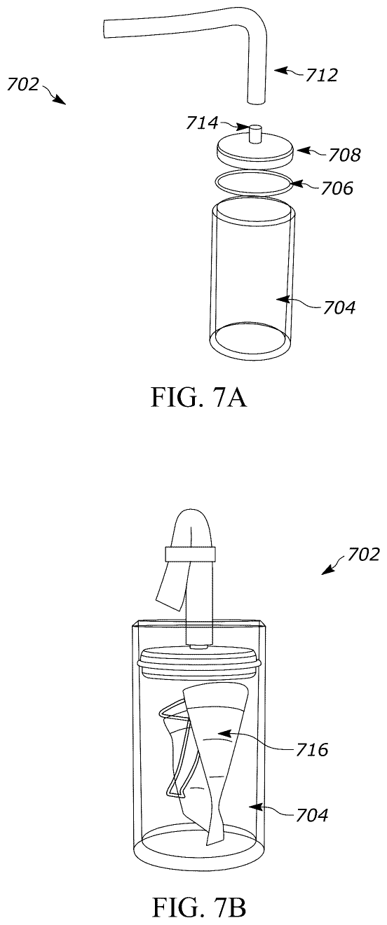

FIGS. 7A and 7B depict another view of an example storage component 702. FIG. 7A depicts an unassembled view of the storage component 702 and FIG. 7B depicts an assembled view of the storage component 702. In particular embodiments, the storage component comprises a receptacle 704, a gasket 706 which goes around the edge of a cap 708, and a tube 712 connected to the cap 708. In particular embodiments, the storage component has minimal gas permeability. In particular embodiments, the receptacle 704 and cap 708 are made of a rigid, inert material, such as stainless steel, glass, or silicone. In particular embodiments, the storage component is sealed with a gasket 706 formed of polytetrafluoroethylene (PTFE) and a cap 708, wherein the cap comprises a flat portion and a jutted portion 714 having a circumference less than that of the flat portion. In particular embodiments, the tube 712 is flexible and formed of PTFE.

In particular embodiments, the storage component is made of Mylar. In particular embodiments, the storage component may be a sealable bag. Although this disclosure describes storage components in a particular manner, this disclosure contemplates storage components in any suitable manner.

In particular embodiments, after the biological sample has been collected in the sample collection component 716, the sample collection component 716 is placed into receptacle 704 and sealed with a cap 708, wherein gasket 706 is located around the circumference of cap 708. In particular embodiments, the cap 708 has a flat portion and a jutted portion 714, wherein the jutted portion has a circumference less than that of the flat proton. In a particular embodiment, the gasket 706 around the cap 708 is operable to keep the sample collection component 716 sealed from the external environment. In particular embodiments, a clinician or the patient can push the cap into the receptacle 704. In particular embodiments, the cap can be only pushed into the receptacle for a set distance due to the interior pressure in the receptacle 704 from the compressed air. In particular embodiments, the receptacle 704 comprises an internal protrusion which functions as a mechanical stop for the cap.

The sample collection kit may also contain an isolation component (not pictured). In particular embodiments, the sample collection component 716 may be a mask that fits tight over the patient's mouth and nose to capture as much exhalant as possible. The exhalant may comprise one or more of liquids, gases, or aerosols from the patient's breath. For example, the majority of the exhalant from the patient may pass through the sample collection component.

In particular embodiments, the collection kit may incorporate a method of user authentication. In particular embodiments, the collection kit may be designed to preserve odors for a long period of time. In particular embodiments, the collection kit assists the user in removing background odors. In particular embodiments, the collection kit indicates to a user when an appropriate amount of biological sample has been collected or authenticate that the user successfully provided a biological sample. In particular embodiments, the user places the collection device containing the biological sample into a hermetically sealed container which preserves the integrity of the biological sample. In particular embodiments, the user seals the sample into a bag, packs it up in a box or envelope, and sends the box or envelope to a testing facility.

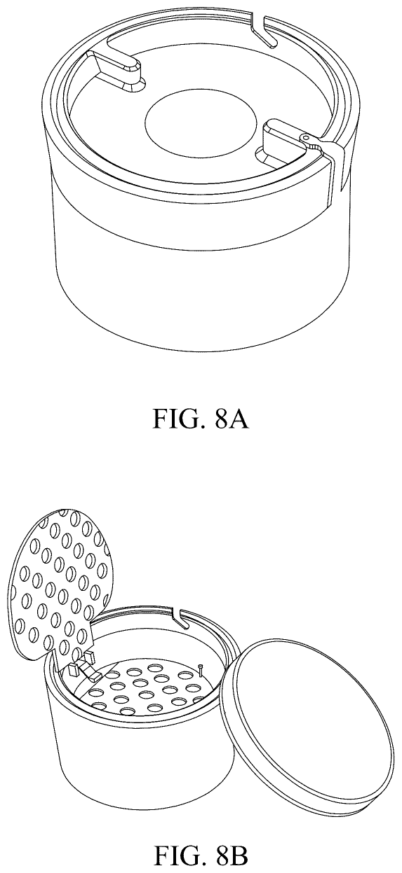

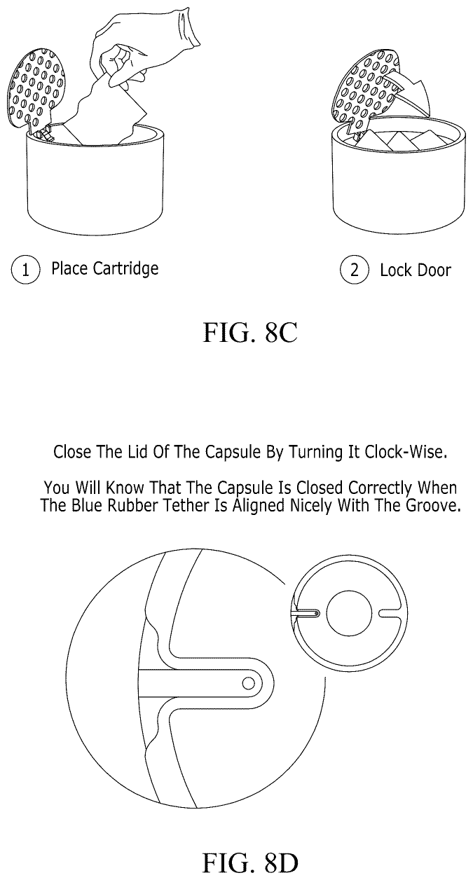

FIGS. 8A-8D illustrate an example storage component. In particular embodiments, the storage component comprises an outer lid and an inner lid. In particular embodiments, the outer lid is detachable, and may be detached by rotating the outer lid. For example, FIG. 8A illustrates an embodiment wherein the outer lid may be detached by rotating the outer lid in a counter-clockwise direction, as indicated by the “OPEN” instruction on the outer lid. In particular embodiments, the inner lid automatically opens when the outer lid is opened. In particular embodiments, the inner lid may be opened by pulling on a part of the inner lid, causing the inner lid to open at a hinge. FIG. 8B illustrates an embodiment wherein the inner lid is open at a hinge, and wherein the outer lid has been removed. FIG. 8C illustrates an example storage component. In particular embodiments, a patient may place the cartridge into the isolation component and close the inner lid. In particular embodiments, the storage component is primarily made of an inert and durable material, such as polysulfone. FIG. 8D illustrates an example storage component. In particular embodiments, after placing the cartridge into the storage component and closing the inner lid, the patient may place the outer lid on the storage component and close the outer lid. In particular embodiments, the storage component comprises a visual indicator, which instructs the user to align the outer lid to a predetermined portion of the capsule portion of the storage component. In particular embodiments, the collected patient sample does not get exposed to the external environment, including during testing.

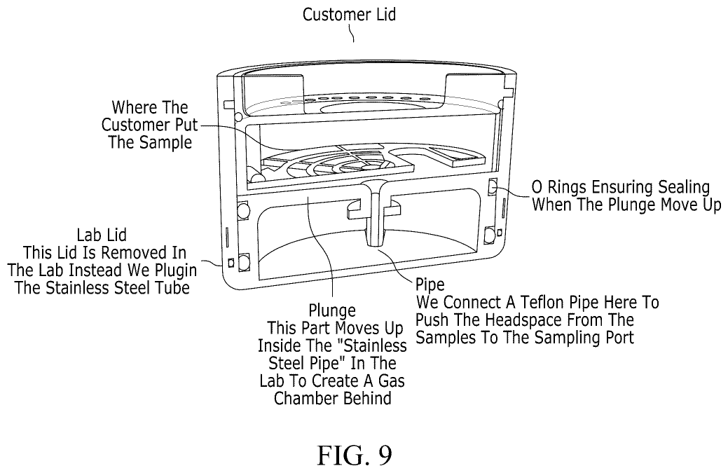

FIG. 9 illustrates an example storage component. In particular embodiments, the storage component comprises the customer lid. In particular embodiments, the storage component comprises a space where the customer puts their sample. In particular embodiments, the storage component comprises a lab lid, which is removed by the lab worker prior to testing the sample. In particular embodiments, the storage component comprises a “plunge,” which may move in an upwards/downwards direction. For example, clean air may be introduced via a pipe, and the plunge may move in a direction away from the compartment containing the patient sample to create a gas chamber. In particular embodiments, the storage component comprises a pipe extending from an opening from the compartment containing the patient sample towards the lab lid. In particular embodiments, the pipe is made of Teflon. In particular embodiments, the pipe enables the LUCID station to push the headspace from the patient sample to the sampling port (sniffing port). In particular embodiments, the pipe is a pre-installed component of the isolation component. In particular embodiments, the pipe is sealed with a valve, and the valve is only removed prior to testing.

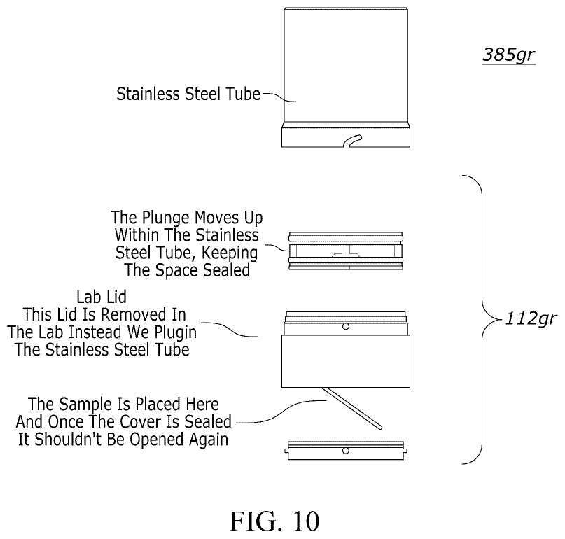

FIG. 10 illustrates an example storage component. In particular embodiments, in preparation for the sample being tested, a lab worker removes the lab lid and attaches an extender to the storage component. In particular embodiments, the extender is a stainless-steel tube. In particular embodiments, the plunge moves up within the extender (e.g., the stainless-steel tube depicted in FIG. 10), while also keeping the space sealed.

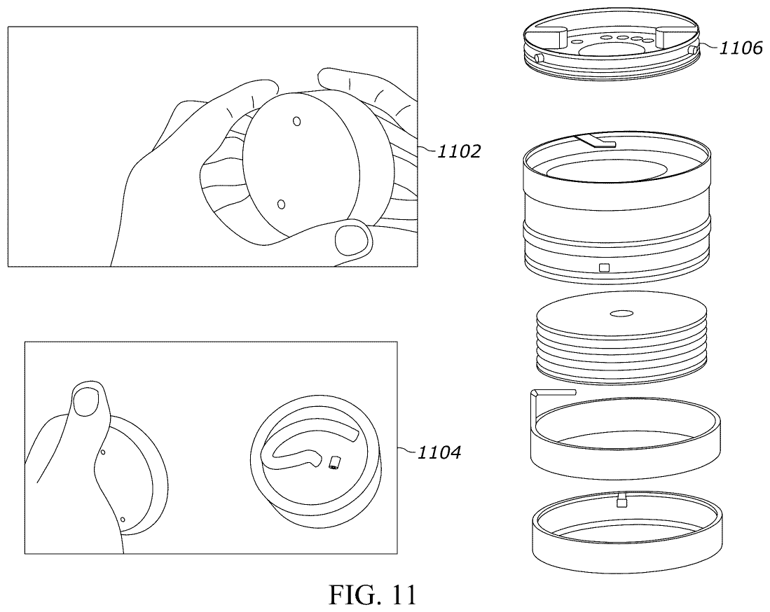

FIG. 11 illustrates an example storage component. In particular embodiments, the storage component has two ends. The first end comprises the outer lid and inner lid discussed previously, wherein the outer lid and inner lid are manipulated by the patient. The second end comprises a “lab lid” to be opened by a lab worker. In particular embodiments, the lab worker may open the lab lid in preparation for testing the patient sample. In particular embodiments, a tube extends from the compartment comprising the patient sample towards the lab end. In particular embodiments, the tube is sealed by a valve. Once the sample is to be tested, the valve is removed, and a lab worker connects the tube to the LUCID station. 1102 depicts a view with the lab lid facing towards the viewer. 1102 depicts a view with the lab lid removed from the storage component. 1106 depicts a view of the storage component, wherein the top of the image shows the customer lid, and the bottom of the image shows the lab lid.

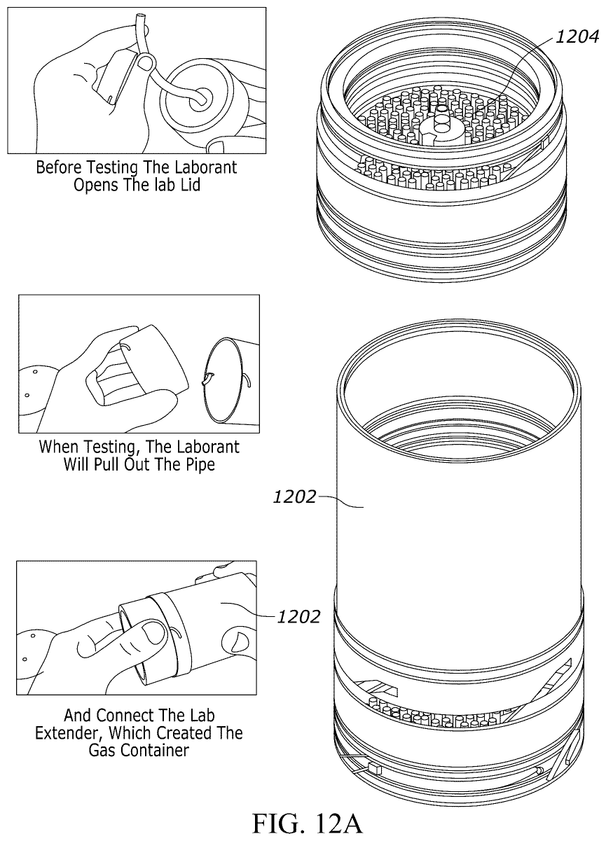

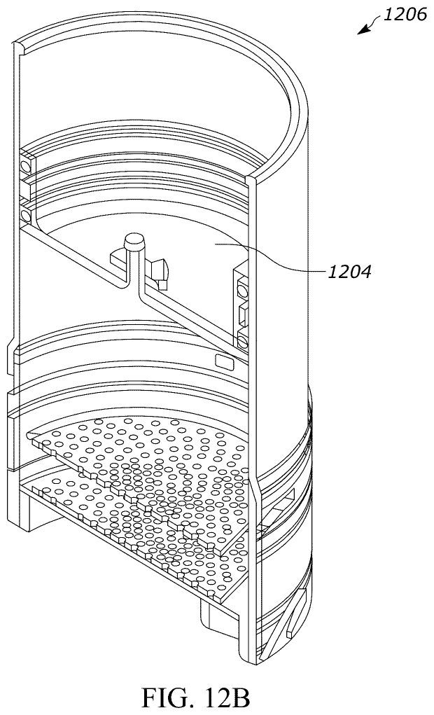

FIGS. 12A and 12B illustrate an example storage component. In preparation for testing, the lab worker (e.g., “laborant”) opens the lab lid for testing and connects an extender 1202 to the storage component, thereby creating a gas container (“container”). The extender is attached to the storage component and allows a piston to move up and down, thereby causing the plunge 1204 to move, with no exposure of the patient sample to the external environment, to create a gas chamber enabling the required headspace. In particular embodiments, a lab worker pulls a plunge of the container up prior to testing the sample to achieve an equilibrium in the headspace. For example, after pulling the plunge up, the lab worker may allow the container to rest for an hour to achieve an equilibrium in the headspace. Then, the lab worker connects the container to the LUCID station, which may start the test by pushing the plunge down to release gas into the LUCID system for testing. 1206 depicts the plunge 1204 at a state wherein the plunge has been pulled upward (e.g., away from the patient sample).

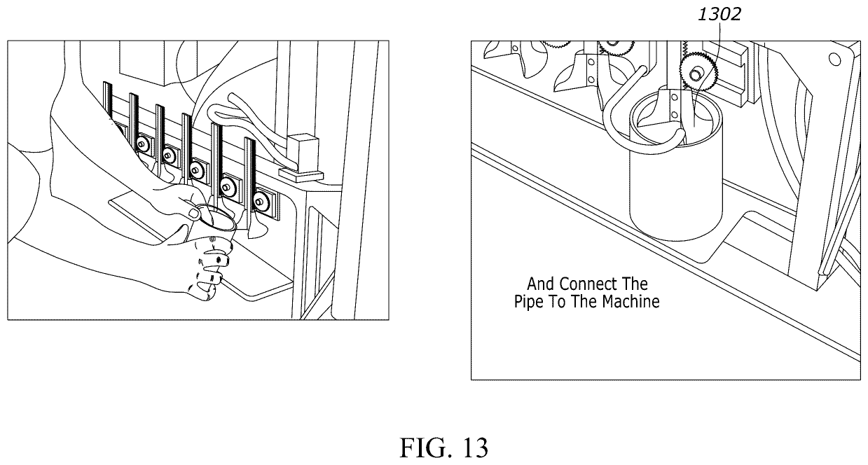

FIG. 13 illustrates an example storage component. In particular embodiments, the storage component may be attached directly to an olfactometer system for testing, without any need for exposing the patient sample to the external environment. In particular embodiments, the olfactometer system comprises one or more pistons 1302 which may control the movement of the plunge. When the sample is ready for testing, air is pushed into the storage compartment. After testing, the lab worker can put the lab lid back on the storage component and send the storage component to storage.

### 5. Test Facility

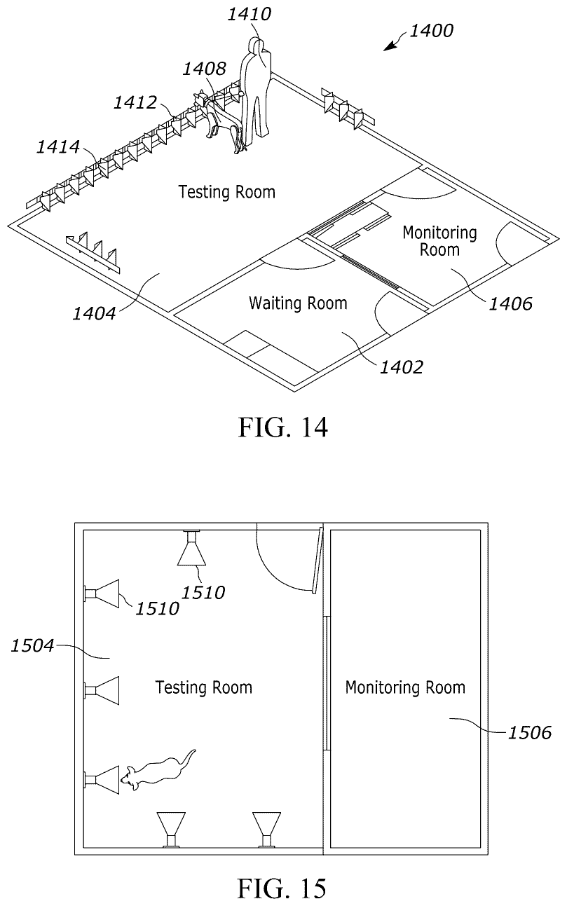

FIG. 14 illustrates an example test facility 1400. In particular embodiments, the test facility 1400 comprises a plurality of rooms: a waiting room 1402, a testing room 1404, and a monitoring room 1406. In particular embodiments, sensors are placed throughout the test facility 1400, and in particular, in testing room 1404 to monitor conditions of the testing room and behaviors, physiological conditions, and neurological conditions of one or more detection animals in the testing room. In particular embodiments, the waiting room 1402 is used for detection animals, and optionally, a human handler 1410, to wait until they are allowed in the testing room 1404. In particular embodiments, the disease-detection system analyzes one or more of: behavior, physiological conditions, or neurological conditions of the detection animal to ensure the detection animal is ready for use in the testing room 1404.

In particular embodiments, the testing room 1404 contains one or more receptacles, including receptacles 1412 and 1414. Each receptacle may contain a biological sample. In particular embodiments, the detection animal, optionally a canine 1408, sniffs each receptacle. In particular embodiments, a separate testing room (not pictured) may be used for particular test(s), such as tests to collect neurological data. As an example, and not by way of limitation, neurological data (e.g., EEG data) may be collected in a different testing room from the testing room 1404 depicted in FIG. 14. In particular embodiments, the biological samples(s) for testing are not placed directed in the testing room 1404; instead, the samples are placed in an olfactometer system connected to the testing room 1404. In particular embodiments, a sampling port of the testing room 1404 is connected via one or more flow paths to an olfactometer system in a separate room which houses the biological samples during testing. In particular embodiments, when the canine identifies a target odor in a receptacle, the canine sits next to the receptacle. In particular embodiments, an automated reward mechanism is located at or near the receptacle. In particular embodiments, the automated reward mechanism provides a reward to the detection animal in accordance with a proprietary reward policy and rewards the animal based on its performance. As an example, and not by way of limitation, the reward may be a food item.

In particular embodiments, the monitoring room 1406 contains a window which allows a person or machine to view the testing room 1404. In particular embodiments, one or more lab workers may be present in the monitoring room 1406 and monitor the screening procedure to ensure the screening is performed according to standard procedures. In particular embodiments, one or more persons in the monitoring room ensures that samples are placed in the correct receptacles in the testing room 1404.

In particular embodiments, a test facility may contain ten testing rooms and be able to facilitate 600 screenings per hour and 1.2 million screenings per year. In particular embodiments, twenty canines are utilized in a test facility. In particular embodiments, one test may be verified by four canines. Although this disclosure describes and illustrates a particular test facility having particular components in a particular arrangement, this disclosure contemplates a test facility having any suitable components in any suitable arrangement.

FIG. 15 illustrates another example test facility comprising a testing room 1504, a monitoring room 1506 and sampling ports 1510.

### 6. Olfactometer System

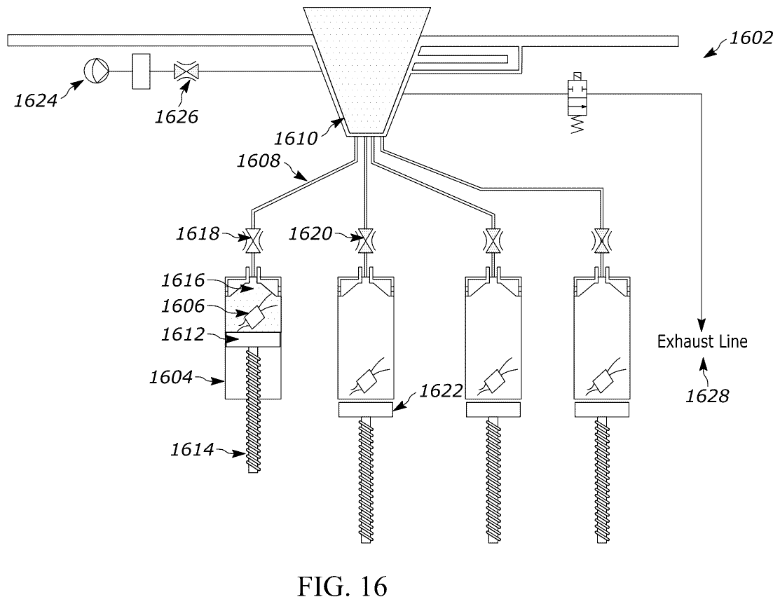

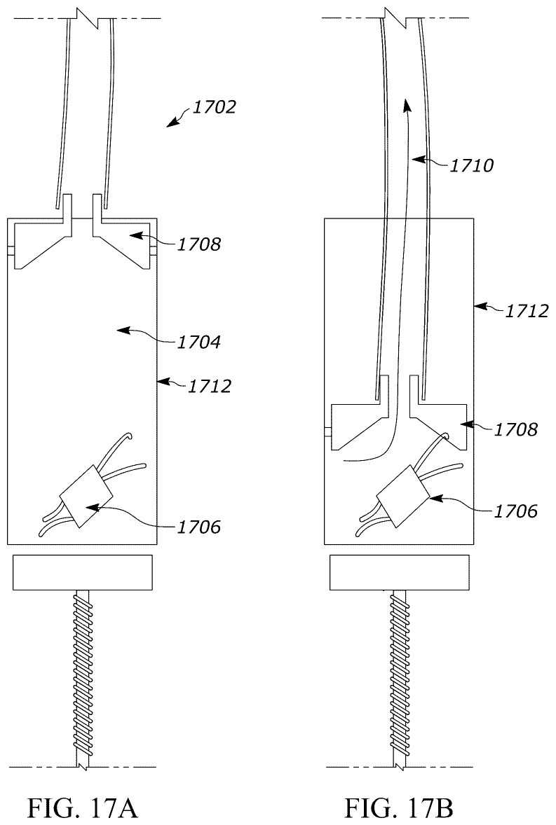

FIG. 16 illustrates an example olfactometer system 1602. The olfactometer system comprises a plurality of receptacles 1604. Each receptacle 1604 is operable to hold a biological sample 1606. The biological sample 1606 may optionally be a mask. Further, a flow path 1608 connects each receptacle to sampling port 1610. Each receptacle has a corresponding piston 1612 and a piston driving portion 1614 which can press the air controllably out of receptacle 1604, thus transporting the odor-soaked air 1616 from biological sample 1606 to the sampling port 1610 via the flow path with zero dilution and in a measurable, repeated, and computed way. The piston driving portion 1614 is coupled to a controller which determines the movement the piston undergoes. For example, the olfactometer delivered a measured amount of odor-soaked air 1616 by driving the piston to a predetermined location, which may be determined by a computing system. In other embodiments, a user may enter a desired pressure for the receptacle to be pressurized to.

Additionally, the biological sample 1606 may be in solid, liquid, or gaseous form. When the biological sample is placed into the receptacle 1604, VOCs which are present in the biological sample are released into the air inside the receptacle 1604. In particular embodiments, the biological sample undergoes an extraction process to maximize the VOCs released from the biological sample. The VOC extraction process is discussed in detail below. This air comprising VOCs from the biological sample (“odor-soaked air”) can be pushed through into the flow path into the sampling port. Accordingly, the olfactometer system is capable of receiving biological samples in solid, liquid, or gaseous states.

### 6.1. VOC Extraction

VOC extraction comprises extracting the VOCs from the biological sample. A VOC extraction process may optionally be performed as part of sample preparation prior to testing. In particular embodiments, VOCs may be extracted through one or more of: heat, pressure, turbulence (e.g. by shaking), or air flow. In particular embodiments, the storage component may withstand temperatures of up to 300° C. In particular embodiments, a biological sample is heated to 24° C.-140° C. In particular embodiments, the VOCs are extracted when the sniff from a detection animal causes turbulence in the biological sample. In particular embodiments, VOCs are extracted, using an olfactometer, by creating a vacuum in a receptacle containing the biological sample and then driving a piston into the receptacle, thereby increasing the pressure in the receptacle. Although this disclosure describes example procedures for VOC extraction, this disclosure contemplates any suitable method for VOC extraction.

In particular embodiments, the testing facility receives a biological sample (e.g., a mask) which is held in a sealed, storage component (e.g., a jar), at a first volume of air. VOCs reside in the biological sample (e.g., a mask), and VOCs which are released from the biological sample are in the air space of the storage component. When the seal of the storage component is opened, air diffusion occurs and the VOCs exit the storage component and may be released via a flow path to a sampling port.

After a desired amount of VOCs are released to a sensor (e.g., a detection animal), by pushing the plunge in the receptacle to extract the sample, the olfactometer system may drive the piston 812 back to its original position, e.g., a position indicated by 1622. When the piston is pulled back, the volume of air is returned back to the first volume and restored to atmospheric pressure. In particular embodiments, the system may add sterile air into the receptacle 804. In particular embodiments, the air pressure required to pull the piston back to its original location (e.g. location 1622 of FIG. 16) requires approximately six times the amount of air required to push the piston on. In particular embodiments, the air pressure required to pull back the piston changes depending on the air volume in the receptacle, wherein the air volume in the container changes over time as the piston is pulled back. In particular embodiments, the location 1622 changes over time. The stream of external sterile air into the container is calculated in a manner to ensure that the pressure on the piston stays constant by increasing the outer air volume stream.

After a period of time, the VOCs is re-released into the airspace of the receptacle. The phenomenon of this re-release of VOCs is an example of solid phase equilibrium. This re-release of VOCs from the biological sample results in the sample being “re-charged” and ready to be used in a next run. In particular embodiments, this “re-charged” sample may be used in a different run—for example, to repeat the run and expose the sample to the same detection animal, or to expose the sample to a different detection animal.

### 6.2. Olfactometer Operation

In certain embodiments, the olfactometer system comprises a plurality of valves, e.g. 1618 and 1620, which may be opened or closed. FIG. 16 depicts valve 1618 in an open position and valve 1620 in a closed position. In an example embodiment, the olfactometer system drives the piston 1612 to cause air from the receptacle to travel through the open valve 1618 to the sampling port 1610 via the flow path 1608. In particular embodiments, the flow rates used to expose the sample to a detection animal are lower than the flow rates used in human applications.

Optionally, a plurality of valves may be open at the same time, and a plurality of pistons each corresponding to a receptacle may be activated at the same time, thus driving a plurality of samples into the sampling port. A benefit of this method of operation is that a plurality of samples (e.g., a “pool”) may be exposed to a detection animal at a first time, thus increasing the efficiency of disease-detection. Upon a determination that a pool of samples has a positive detection event, the olfactometer system can individually expose each biological sample to the detection animal to determine the one or more biological samples which contain cancerous VOCs.

In particular embodiments, two or more biological samples may be mixed to create a new sample for training or maintenance purposes. In particular embodiments, the olfactometer system may expose a plurality of samples to a detection animal for training. In particular embodiments, a mixed sample may be created by lab personnel. In particular embodiments, one or more known biological samples (e.g., known biological samples with lung cancer) may be mixed for training.

In certain embodiments, there are one or more sensors proximate to the sampling port. Example sensors include: a biosensor such as a detection animal (e.g., a canine), a biochemical sensor, or electrical sensors. In particular embodiments, a sensor proximate to the sampling port can measure the total and/or specific amount of VOCs which is delivered to the sampling port. This sensor simultaneously has a quality control function by ensuring that the correct amount of VOCs, and a correct amount of odor-soaked air, have been delivered to the sensor(s). In particular embodiments, sensors may comprise one or more gas sensors, including a chemical or electrical sensor that can measure a total amount of VOCs or detect the presence of a particular VOC. In particular embodiments, the gas sensor measures the volume of the exposed sample, the exposed sample comprising both VOCs and air. In particular embodiments, the gas sensor can detect a quality or quantity of an inorganic gas, the inorganic gas which is correlated to a quality or quantity of a biological sample. In particular embodiments, data from one or more gas sensors is input into one or more ML-models for calculating a confidence score.

In particular embodiments, the olfactometer system 1602 performs a cleaning cycle using an automated process, resulting in increased efficiency and throughput of sample testing. A cleaning cycle is performed using gas (e.g., compressed air) from a gas source 1624. The gas source 1624 flows through valve 1626. FIG. 16 depicts valve 1626 in a closed state. However, during an example cleaning cycle, the system may close the valves between the sampling port 1610 and the receptacles (e.g., 1604), and open valve 1626 to run clean air through the system. The clean air flushes VOCs out of the sampling port and follows a path ending at the exhaust line 1628.

FIGS. 17A and 17B illustrate another example embodiment 1702 of a receptacle comprising a piston. FIG. 17A depicts an embodiment wherein the odor-soaked air 1704 is not being pushed out of the receptacle 1712. The odor-soaked air 1704 comprises VOCs from biological sample 1706. FIG. 17A depicts piston 1708 in a non-activated position. FIG. 17B depicts the piston 1708 in an activated position. While in an activated position, the piston is driven into the receptacle 1712, thereby causing the odor-soaked air from the sample to travel to the sampling port through flow path 1710. The odor-soaked air may be controllably pushed out of the receptacle 1712, thereby causing a predetermined amount of air to travel to the sampling port, with zero dilution.

It is evident from FIGS. 17A and 17B that the piston 1708 is at a first location in the receptacle 1712 while in a non-activated state, and at a second location in an activated state.

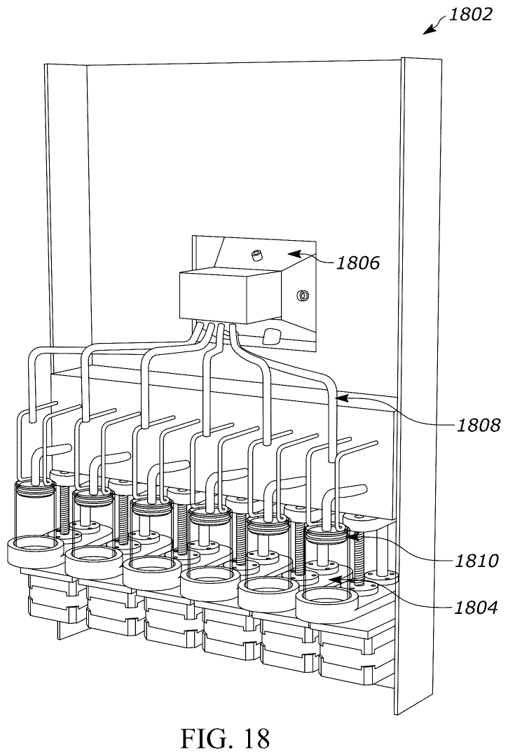

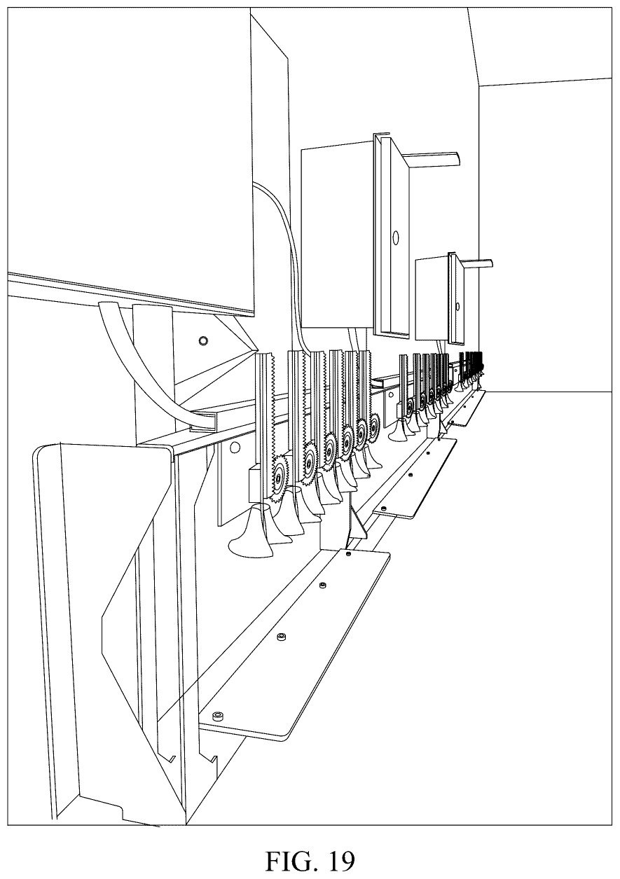

FIG. 18 depicts an example view of an olfactometer system 1802. In an example embodiment, the receptacles 1804 operable to hold a biological sample are located in a first room and the detection animal operates in a second room. A sampling port 1806 is contained in the second room, and the sampling port is connected via a plurality of flow paths 1808 to receptacles in the first room. As described in the example embodiments above, odor-soaked air from the receptacles 1804 may be delivered to a sampling port by driving a piston 1810 into the receptacle, thereby causing a predetermined amount of gas to travel through a flow path 1808 to the sampling port.

In particular embodiments, the receptacle 1804 is formed of inert material such as stainless steel. In particular embodiments, the sealed receptacle may be connected to the olfactometer system without exposing the biological sample to the environment. In particular embodiments, a tube connected to the storage component may be attached to a fitting of the olfactometer system.

FIG. 19 depicts an example view of a testing facility. A view of the laboratory side, (where the test samples are located) is depicted by the figure.

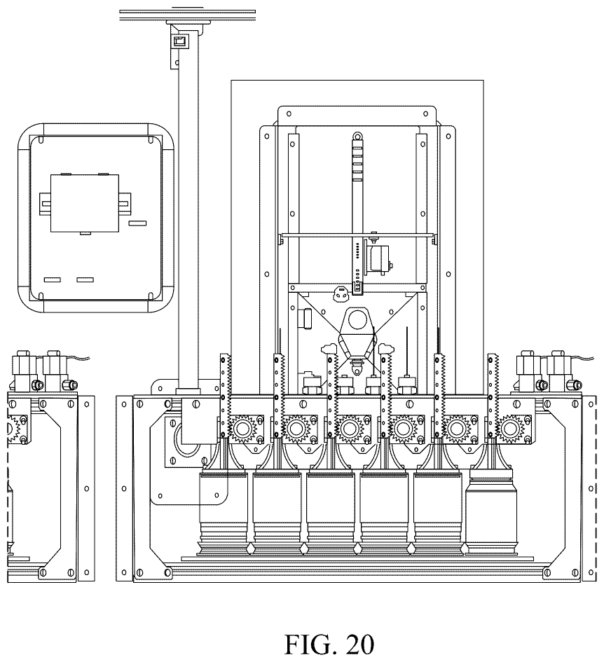

FIG. 20 depicts an example view of an olfactometer system. In particular, the figure depicts the olfactometer system in a state wherein the first five containers (counting from the left) have been tested. The tested containers are in a “squeezed” state wherein the piston has squeezed the air into the sampling port for testing. The right-most container is in an “un-squeezed” state, indicating that the sample inside has not been exposed to a canine for testing.

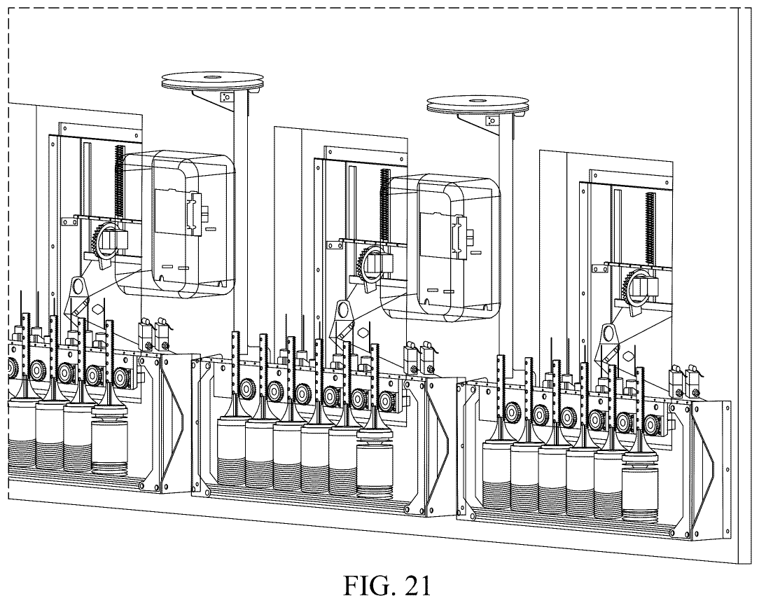

FIG. 21 depicts an example view of an olfactometer system.

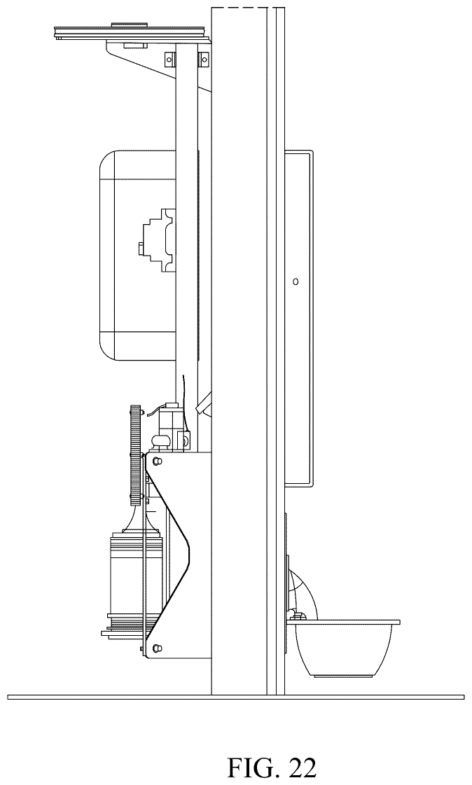

FIG. 22 depicts an example view of an olfactometer system. In particular, the figure depicts the lab portion on a first side of a wall, and the “testing” portion on the second side of the wall. As used herein the “testing” portion refers to a wall of the room where a canine performs its detection function.

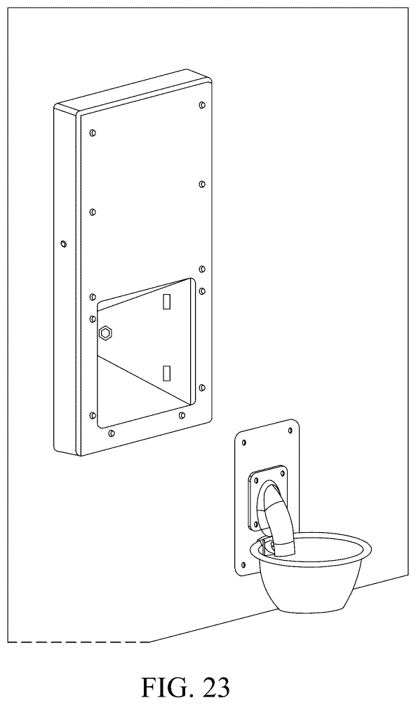

FIG. 23 depicts an example view of a sampling port. In particular embodiments, a treat is dispensed to the canine at certain times during a test run. In particular embodiments, the treat is disposed at a location close to the sampling port. In particular embodiments, the time at which to dispense the treat is determined by the LUCID platform, which may take into account one or more of: the canine's behavior, or the predicted accuracy of the canine's detection of one or more particular samples.

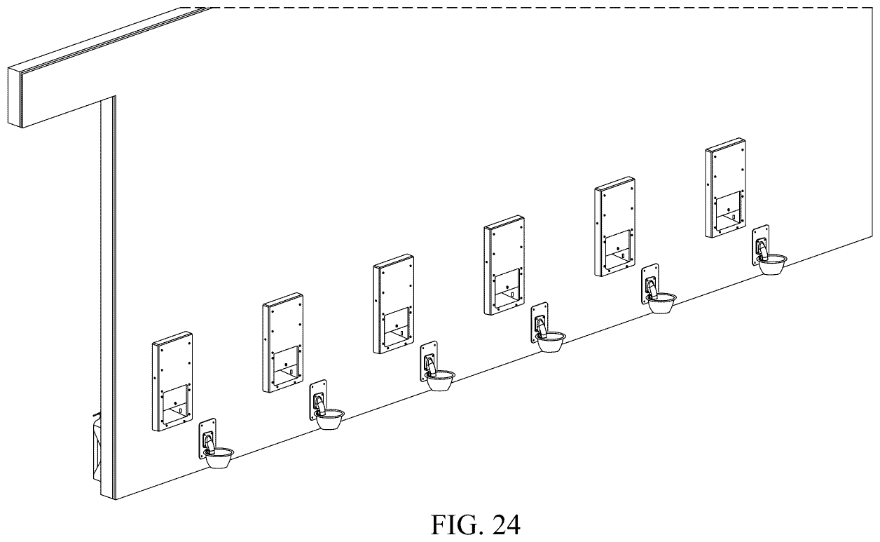

FIG. 24 depicts an example view of sampling ports in a testing room.

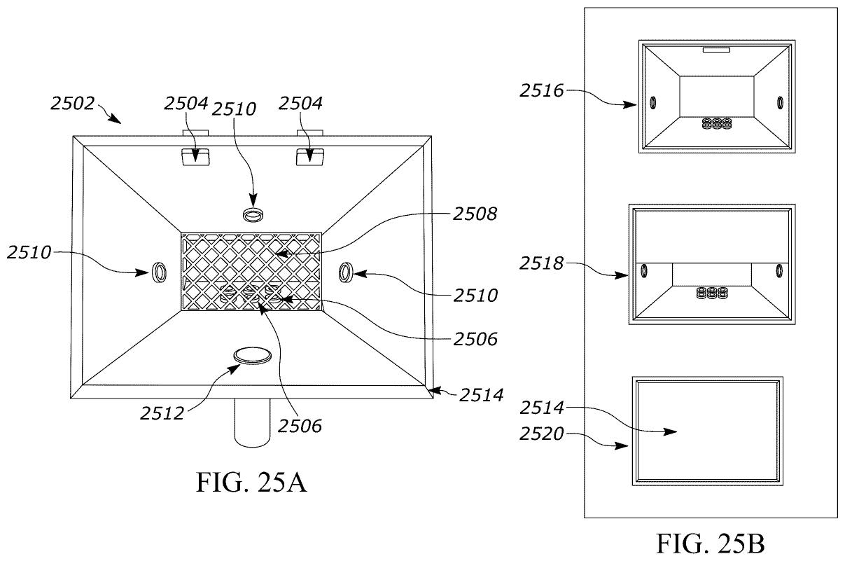

FIGS. 25A-25B show views of a sampling port. In FIG. 25A, the sampling port 2502 comprises two infrared sensors 2504, which are operable to measure the length of a sniff of the detection animal. In particular embodiments, the ML system interprets a sniff of at least 200 milliseconds (ms) as constituting a valid sniff. In particular embodiments, if the detection animal removes its nose early, e.g., before a predetermined time interval of 200 ms for example, then the flow path from the biological sample to the sampling port is stopped. In particular embodiments, if the detection animal sniffs the sample for more than a predetermined time, e.g., 300 ms, then the olfactometer system pushes more odor from the receptacle holding the biological sample, to the sampling port. The olfactometer system transports odor from the receptacle holding the biological sample, to the sampling port, through low pressure inlets 2506. FIGS. 25A-25B depict six low pressure inlets behind a replaceable grill 2508. Although this disclosure describes a system with a particular number of low-pressure inlets, this disclosure contemplates a system with any suitable the number of low-pressure inlets, and in particular embodiments, the number of low pressure inlets correspond to the number of receptacles operable to hold a biological sample. The replaceable grill 2508 serves to prevent the detection animal from directly touching the low-pressure inlets 2506. The olfactometer system also comprises a plurality high-pressure cleaning inlets 2510. The high-pressure cleaning inlets 2510 inject clean air into the sampling port to clean the sampling port between runs. Exhaust port 2512 provides a mechanism from removing air from the sampling port. The sampling port further comprises a mechanized door 2514, the operation of which is depicted in FIG. 25B.

FIG. 25B depicts a mechanized door 2514 of the sampling port. The mechanized door 2514 may be opened or closed. In particular embodiments, the mechanized door remains closed unless active testing is being formed. The closed door prevents contaminants from the external environment or the laboratory environment from traveling inside the sampling port. 2516 depicts the mechanized door 2514 in a fully open state, 2518 depicts the mechanized door 2514 in a half open state, and mechanized door 2520 depicts the mechanized door 2514 in a fully closed state.

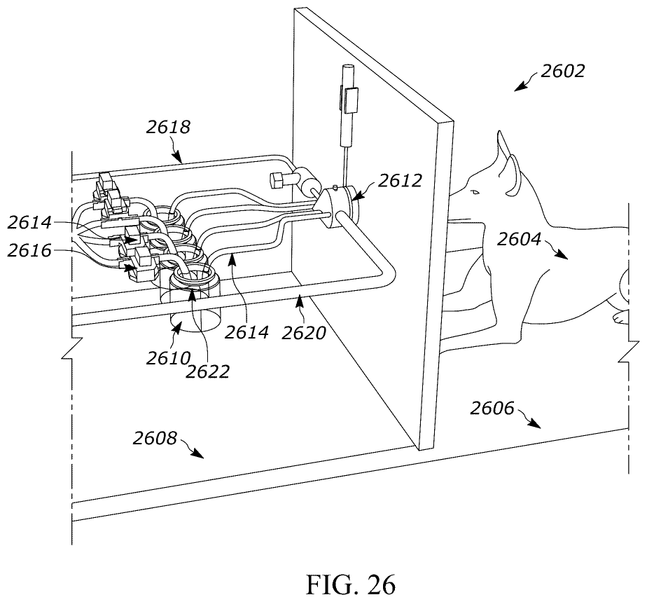

FIG. 26 depicts an example view of an olfactometer system 2602. In particular embodiments, the detection animal 2604 is in a first room 2606, a sampling port (not pictured) is located in the second room 2608, and the receptacles 2610 are in a second room. An example portal to the sampling port is depicted as portal 2612. The receptacles are connected to the sampling port via a plurality of flow paths 2614. In an example embodiment, the physical separation between the first room and the second room enables the clinical facility to continuously load biological samples in the second room 2608 while the detection animal performs continuous testing in the first room 2606.

In an example operation, a biological sample is placed into each receptacle 2610, and the receptacle 2610 is attached to the olfactometer system 2602. In particular embodiments, the olfactometer system runs a cleaning step. In particular embodiments, during the cleaning step, valves (e.g. 2616) to the receptacles are in a closed position, and air is flushed through flow paths 2618 and 2620, as well as through the portal 2612 to the sampling port. In particular embodiments, air passes through or more of an active carbon filter or a humidity trap filter before it is pushed into the olfactometer system.

During a run, one or valves 2616 may be opened. For example, during a test comprising pooled samples, a plurality of valves 2616 may be opened to allow odor-soaked air from a plurality of receptacles to be delivered to the sampling port. In other embodiments, only one valve is opened at each time. Further, during a run, the piston 2622 is driven into the receptacle, thereby forcing odor-soaked air out of the receptacle and through the flow path.

### 7. Disease-Detection System

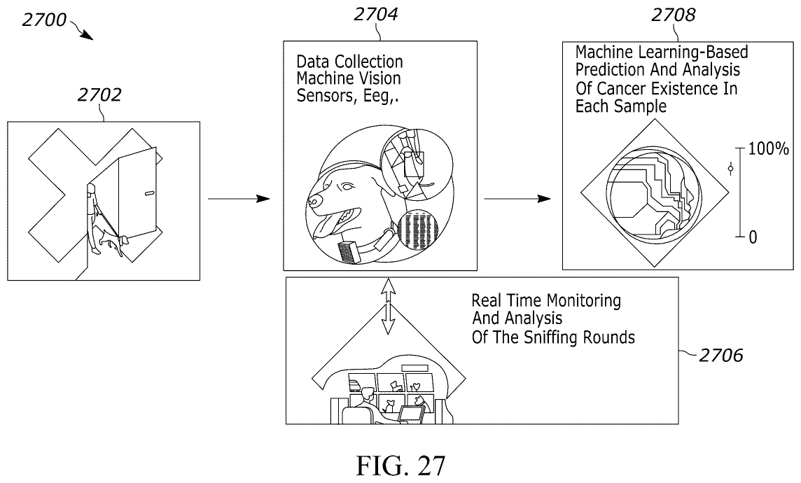

FIG. 27 illustrates an example method 2700 of the disease-detection system, which comprises a data collection step 2704, a real-time monitoring and analysis step 2706, and a ML-based prediction and analysis step 2708. In particular embodiments, the disease-detection system may further comprise one or more additional computing components, including a monitoring component and an operational component.

The method 2700 may begin at step 2702 with a detection animal entering a testing room. In one embodiment, the testing room contains a plurality of samples, including biological samples from one or more patients, and one or more service samples. In another embodiment, the testing room contains one or more sampling ports which are coupled to one or more receptacles containing one or more biological samples.

At step 2704, the disease-detection system collects data from one or more sensors comprising: one or more behavioral sensors, one or more physiological sensors, one or more neurological sensors, or one or more operational sensors. In particular embodiments, the sensors measure one or more of: animal behavior, animal physiological patterns, or animal neurological patterns.

In particular embodiments, behavioral sensors collect data on a behavior of the detection animal. In a particular embodiment, behavior may include a body pose of the detection animal. As an example, and not by way of limitation, body poses include, but are not limited to, standing at or next to the sampling port, sitting next to the sampling port, or looking at a handler. As an example, and not by way of limitation, a body pose may include a position of the detection animal relative to the sampling port. As an example, and not by way of limitation, animal behavior may include: repeatedly sniffing a particular receptacle or long sniffs at a particular receptacle, which may indicate that the detection animal is indecisive as to the status of the biological sample. Animal behavior may include the amount of time an animal investigates a particular receptacle, and the amount of time it takes for an animal to indicate it found a target odor after investigating a receptacle. Animal behavior may also include the speed at which the detection animal walks between sampling ports and acceleration data associated with the detection animal walks between the sampling ports. In particular embodiments, data is collected on one or more of: the duration of a sniff (e.g. the length of time a detection animal sniffs the biological sample), the number of repeated sniffs, the time between a sniff and a signal, or the time it takes the canine to signal. In particular embodiments, animal behavior comprises features of a sniff which are measured by one or more audio sensors. As an example, and not by way of limitation, features of a sniff comprise one or more of a sound, intensity, or length of a sniff. Responses may include monitoring of sitting (or not sitting), changes in face gestures, change in body position, neurological features of the detection animal, or other suitable features observable in the detection animal. As an example, the ML-model may analyze sitting (or not sitting) responses and/or changes in landmarks relating to face gestures, body position, neurological features of the detection animal during a sniffing run, or other suitable features observable in the detection animal. Although this disclosure describes using the above exemplary inputs into a ML-model, this disclosure contemplates using any suitable type of input into a ML-model.

In particular embodiments, environmental sensors collect data on one or more conditions of the testing room, including at locations near the sampling port. As an example, and not by way of limitation, environmental sensors are operable to receive data associated with the testing room and/or the sampling port(s), such as the temperature, humidity, noise level, air flow, and air quality of the testing room or the sampling port(s).

In particular embodiments, the data collection step 2704 comprises collecting data from one or more physiological sensors comprising one or more of: heart rate monitor, heart rate variability monitor, temperature sensor, galvanic skin response (GSR) sensor, sweat rate sensor, or a breath rate sensor.

In particular embodiments, the data collection step 2704 comprises collecting data from one or more neurological sensors comprising one or more of: one or more electroencephalogram (EEG) sensors, one or more functional near-infrared spectroscopy (fNIR) sensors, one or more functional magnetic resonance imaging (fMRI) scanners, one or more electromyography imaging (EMG) scanners, or one or more magnetic resonance imaging (MRI) scanners.

In particular embodiments, the data collection step 2704 comprises collecting data from operational sensors. In particular embodiments, the operational sensors comprise one or more of: sensors in the olfactometer, sensors for animal management (e.g., a RFID card which identifies a particular canine), and sensors for sample management (e.g., a QR code scanner which scans a unique QR code associated with each biological sample).

In particular embodiments, the data collection step 2704 comprises receiving non-behavioral data such as the family medical history, patient medical history, patient age, patient gender, or patient demographical data. As an example, and not by way of limitation, patient medical history may include whether or not the patient smokes, the patient's alcohol consumption, or other lifestyle factors.

The method 2700 may continue at step 2706 wherein a person or a machine performs real-time monitoring and analysis of one or more of the behavioral sensors, physiological sensor, or environmental sensors, during one or more of the rounds of animal investigation. In particular embodiments, the real-time monitoring and analysis may be done on one detection animal; in other embodiments, the real-time monitoring and analysis may be done on a pack of detection animals. In particular embodiments, each detection animal has a monitoring algorithm (e.g., an ML-model operable for a monitoring function) calibrated to that particular detection animal. In particular embodiments, an animal investigation is a sniffing round in which a canine sniffs the receptacles in the testing room. In particular embodiments, a human or machine monitors the testing to ensure standard operating procedures are followed by the detection animal and/or its human handler. In particular embodiments, step 2706 includes one or more actions performed by a computing component of the disease-detection system. As an example, and not by way of limitation, the computing component may comprise a real-time monitoring program which monitors a condition (e.g., temperature) of the testing room and alerts the lab manager immediately upon detection of an out-of-range condition. As used herein, “lab manager” refers to one or more persons responsible for setting up a run (either physically or through a machine), or overseeing a run.

In particular embodiments, the disease-detection system monitors parameters and provides alerts for certain parameters in real-time regarding certain abnormalities (e.g., an environmental abnormality or a behavioral abnormality) or failures within the test procedure. As an example, and not by way of limitation, real-time monitoring and analysis comprises receiving and analyzing environmental sensor data (e.g. temperature, humidity range, etc.), and alerting a lab manager if one or more of predetermined environmental data is out of range. As an example, and not by way of limitation, the system may alert a lab manager upon an indication that a sensor is not functioning properly. As an example, and not by way of limitation, the real-time monitoring and analysis comprises monitoring a particular action of a detection animal (e.g., a sniff at a sampling port) to determine whether the action meets a predetermined criteria (e.g., a duration of a sniff).

In particular embodiments, the system monitors the behavior of the detection animal for behavioral abnormalities (e.g. a long duration of a sniff without any positive or negative indication of a disease state). In particular embodiments, if the measured action does not meet a predetermined criteria, the system provides an alert to the lab manager. In particular embodiments, step 2706 comprises monitoring that the received sensor data is valid. In particular embodiments, step 2706 comprises monitoring animal behavior for any drift of animal performance during a test run. In particular embodiments, behavioral drift may be monitored by either a ML-model or a computing component of the disease-detection system. In particular embodiments, the parameters may further include a physiological condition of a dog, such as one or more of: a heart rate, a heart rate variability, a temperature, a breath rate, or a sweat rate. The parameters may further include sample storage conditions, such as temperature and humidity. In particular embodiments, the system may alert the lab manager in real-time, after a positive detection event. In particular embodiments, the disease-detection system comprising the biological samples, the detection animals, the laboratory facilities, and the storage facilities are continuously monitored, and alerts are pushed to a person when one or more parameters is out of range. In particular embodiments, if an alert affects a clinical test, an alert pops up on the monitoring screen, requiring a lab manager to take action.

In particular embodiments, the disease-detection system monitors every sniff of the detection animal and based on predetermined thresholds set as a valid sniff (e.g., a time period of 200 ms), the system provides alerts in real-time when a sniff does not meet the predetermined threshold.

The disease-detection system records certain activities performed in the sniffing rooms. For example, the activities may include the behavior of the handler of the detection animal. Further, the disease-detection system records all signals received from the canines, which may include physiological data from one or more sensors and animal behaviors such as an animal pose.

In particular embodiments, the real-time monitoring and analysis 2706 ensures that each test run is performed under predetermined conditions (e.g., within a predetermined range of temperature, light level, sound level, air particulate level, wherein the behavior of the detection animal meets a predetermined criteria, wherein there are no behavioral abnormalities, etc.), but data from the real-time monitoring and analysis 2706 is not directly input into the ML-based prediction and analysis 2708.

The method 2700 may continue at step 2708 wherein the disease-detection system uses one or more ML-models to perform ML-based prediction(s) based on one or more of the: behavioral data, physiological data, neurological data, or patient data received from data collection step 2704. As an example, but not by way of limitation, a ML-body may receive animal behavior data, e.g. a body pose, and patient data as an input.

In particular embodiments, the disease-detection system comprises one or more ML-models. In particular embodiments, the one or more ML-models include: one or more ML-models for a particular detection animal (e.g., a dog-specific ML-model), one or more ML-model for a plurality of detection animals (e.g., a dog pack-specific ML-model), one or more test stage-specific models (e.g., a ML-model for a first stage of a test, a ML-model for a second stage of a test), one or more ML-models trained on disease states (e.g. a positive or negative determination of cancer), one or more ML-models trained on cancer types (e.g., breast cancer, lung cancer, colon cancer, prostate cancer), one or more ML-models trained on cancer stages (e.g., stage 1, stage 2, stage 3, or stage 4), or one or more neurological-based ML-models, or one or more monitoring ML-models (e.g., monitoring the behavioral drift of a detection animal). In particular embodiments, the one or more ML-models may receive one or more of: behavioral data, physiological data, neurological data, or patient data. In particular embodiments, a test run comprises a plurality of stages. As an example, and not by way of limitation, a first stage of a test may comprise a plurality of detection animals performing a run. As an example, and not by way of limitation, a second stage of a test may comprise aggregating the scores from the first stage of the test. Although this disclosure describes and illustrates particular steps of a test, this disclosure contemplates any suitable steps for a test, the steps which may occur in any suitable order.

In particular embodiments, once the test run has finished, the disease-detection system may give recommendations as for the lab results of each sample participating, with the ability of the lab personnel to intervene and alter the results based on the data they are presented with. In particular embodiments, the ML-based disease-detection model provides both a lab result (e.g., a ML-based result of a disease state and an associated confidence interval) as well as the dog result prediction (e.g., a particular behavior of a dog which indicates a particular disease state).

In particular embodiments, the ML-based disease-detection model generates feature representations based on one or more of behavioral responses, physiological responses, or neurological responses of the detection animal exposed to a biological sample. In particular embodiments, the ML-based disease-detection model further receives patient data. In particular embodiments, the one or more ML-models are created through offline learning. In particular embodiments, the one or more ML-models are created through online learning. In particular embodiments, the ML-based disease-detection model may store black box features without any interpretation.

In particular embodiments, one or more ML-based disease-detection models are trained on indications or signals of a detection animal associated with a biomarker (e.g., a particular scent of a VOC). As an example, and not by way of limitation, indications from a detection animal may comprise one or more of: a sitting position, a lying position, or looking at the animal handler to indicate a positive disease-detection event. As an example, and not by way of limitation, signals such as heart rate, heart rate variability, and temperature of the detection animal may change upon different sample indications as a result of the anticipation for a reward. Furthermore, signals generated by neurosensory collection (e.g., by EEG, fNIR, fMRI, or MIR) may change upon one or more of: a positive or negative cancer state, a type of a cancer, or a stage of a cancer.

In particular embodiments, a validation step is performed to measure the performance of the one or more ML-models by comparing the determination outputted by the ML-based disease-detection model, with the known disease state of a training sample. In particular embodiments, the ML-based disease-detection model is validated by: exposing one or more training samples to one or more detection animals, wherein each of the training samples has a known disease state, receiving sensor data associated with one or more detection animals that have been exposed to the training sample, calculating one or more confidence scores corresponding to one or more disease states associated with the training samples, and determining a number of inferences by the ML-based disease-detection model that are indicative of the particular disease state.

In particular embodiments, the known disease state of the training sample may be obtained through a liquid biopsy. As an example, and not by way of limitation, the discrepancy between the target disease state and the disease state detected by the ML-model is measured, and the training method described herein is re-performed until a predetermined number of iterations is reached or until the value associated with the discrepancy reaches a predetermined state. In particular embodiments, the system iteratively updates the parameters of the ML-based disease-detection model using an optimization algorithm based on a cost function, wherein the cost function measures a discrepancy between the target output and the output predicted by the ML-based disease-detection model for each training example in the set, wherein the parameters are repeatedly updated until a convergence condition is met or a predetermined number of iterations is reached. In particular embodiments, the system outputs a trained ML-based disease-detection model with the updated parameters.

In particular embodiments, a positive disease-detection event may result in confirming the positive disease-detection of the biological sample through another method, such as by a genomic test. In particular embodiments, the additional test is performed upon a determination that the confidence score is below a predetermined threshold. In particular embodiments, the genomic test is performed using a liquid biopsy from the patient.

In particular embodiments, an EEG device worn by a detection animal may be used as an additional verification step. In particular embodiments, the EEG data indicates the origin of cancer (e.g. whether the cancer is from the breast or the lung). In particular embodiments, a neurological-based ML-model analyzes the EEG response of a detection animal after it has been exposed to a particular odor.

In particular embodiments, one or more neurological-based ML-models are developed based on a detection animal's neurological response to a target odor. For example, one or more ML-models may be developed to detect a disease state (e.g. positive or negative cancer state), a cancer type, or a cancer stage. In particular embodiments, a neurological-based ML-model may receive data comprising one or more of behavior data, physiological data, or patient data. In particular embodiments, non-neurological data, such as operational data associated with the olfactometer (e.g., a start and end time of odor release), behavioral data, and physiological data (e.g., a heart rate) are also collected during an EEG or other neurological-based test. In particular embodiments, the detection animal is not trained for an odor detection task. In particular embodiments, the neurological-based ML-model receives neurological data (e.g., EEG data), as well as data from an olfactometer. In particular embodiments, data from the olfactometer comprises a timeline indicating the time(s) that a particular odor is exposed to the detection animal. In particular embodiments, the neurological-based ML-model receives data from an accelerometer worn by the detection animal during testing (and including during the exposure event). In particular embodiments, the neurological-based ML-model receives behavioral data and physiological data from the sensors described herein.

In particular embodiments, the olfactometer comprises a sampling port which is coated with Teflon, or a Teflon-based material to facilitate deodorization and reduce signal interference from conductive materials such as stainless steel. In particular embodiments, the sampling port may be formed of glass. In particular embodiments, the testing area is formed of a Teflon-based material. In particular embodiments, the detection animal is on a Teflon-based platform (e.g., a bed of a detection animal) during testing. Although this disclosure describes suitable materials for an olfactometer system in a particular manner, this disclosure contemplates any suitable materials for the olfactometer system, and in particular, the sampling port.

In particular embodiments, the neurological response comprises a trend in an EEG. In particular embodiments, a neurological-based ML-model may be trained on correlations between a detection animal's neurological response and a target odor. In particular embodiments, the neurological-based ML-model outputs one or more of: a positive or negative state (e.g., a positive or negative cancer indication), a cancer type, or a cancer stage. In particular embodiments, neurological data is input into the ML-based disease-detection model described herein.

In particular embodiments, the ML-based disease-detection model calculates a confidence prediction interval according to a statistical calculation. Additionally, the ML-model estimates the probability of cancer for the sample, along with its confidence prediction interval. Based on it, the algorithm simplifies these measurements to: predicted disease state and its confidence score.

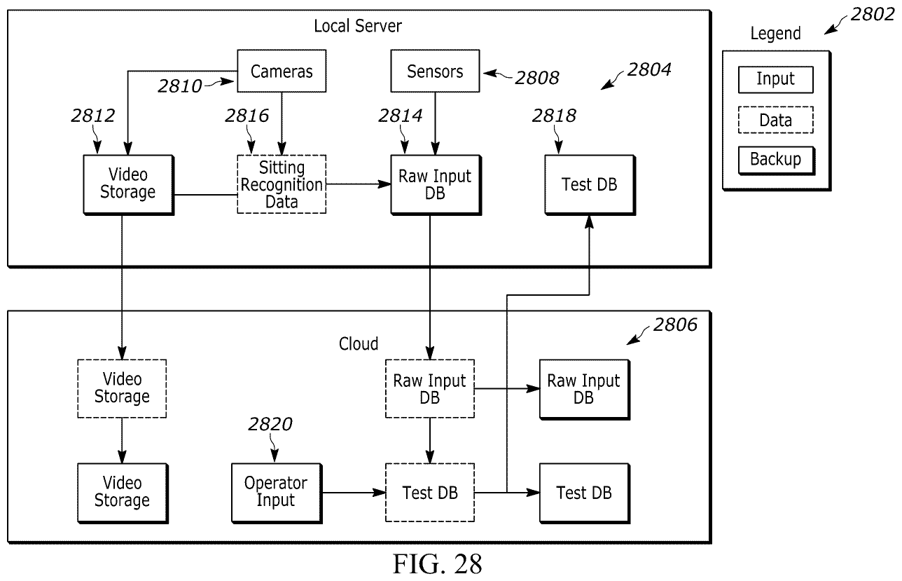

FIG. 28 depicts an example data flow of the disease-detection system 2802. The system comprises data stored on a local server 2804 and a cloud 2806. In particular embodiments, sensor data, video data, and operator input is streamed into the system in real time. In particular embodiments, operator input 2820 is performed by a lab manager. For example, sensor data from one or more sensors 2808 may contain one or more of: sniff events for each detection animal and the associated sampling port(s), movements (e.g., a walking speed or an acceleration) of the detection animals, and laboratory conditions. The sensors 2808 may comprise one or more of the behavioral, physiological, or neurological sensors described herein. Further, camera/video data from one or more cameras 2810 may comprise information related to animal behavior and animal pose. For example, an animal pose may comprise a sitting or standing position of an animal. It may also comprise whether the animal looks at its handler. Animal behavior may comprise sniffing behaviors or the animal behavior in the lab (e.g., the speed at which the animal walks). Videos are temporarily stored at a video storage location 2812 at a local server and before they are transferred to the cloud 2806. In particular embodiments, data comprising one or more of: environmental data, operational data, and lab manager inputs (e.g., run data), is also stored on the cloud 2806. In particular embodiments, operator input 2820 is stored on the cloud 2806. In particular embodiments, operator input 2820 comprises one or more of: family medical history, patient medical history, patient age, patient gender, or patient demographical data. In particular embodiments, a sitting pose is indicative of a positive detection event, and corresponding sitting recognition data 2816 is input into raw input database 2814. The system may further receive inputs into raw input database 2814 which comprise sensor data discussed herein, such as from one or more of: behavioral sensors or physiological sensors. In particular embodiments, the lab manager may input information regarding demographic data of the detection animal, such as the age, sex, or breed of the detection animal. In particular embodiments, the lab manager may input information regarding the patient, such as one or more of: family medical history, patient medical history, patient age, patient gender, or demographical data of the patient. The inputs may further comprise information about the number of detection rounds a detection animal has performed. The rounds data comprise the number of exposures of the detection animal to a biological sample.

Tests database 2818 comprises data about the resources (e.g., the samples, dogs, lab manager, animal handler, lab manager, and the tests). The tests database is formed by processing the raw input data as well as the data input by a user (e.g., a lab manager).

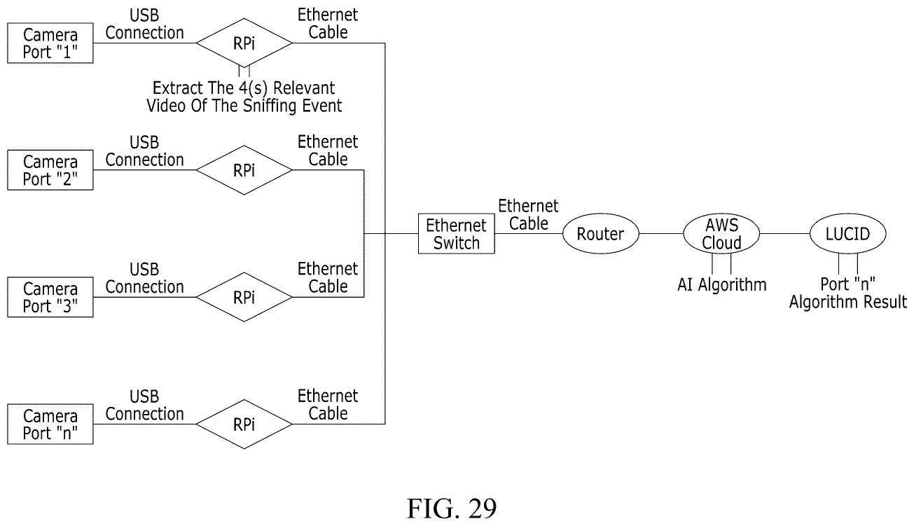

FIG. 29 depicts another example computing architecture for analyzing unconditioned facial gestures comprising a plurality of cameras for capturing facial gestures of the dogs. Each camera is disposed in operable communication with a computer (e.g., Raspberry Pi, RPI), which in turn is in operable communication with the LUCID platform. In particular embodiments, a USB connection enables operable communication between a camera and the RPI. In particular embodiments, an ethernet cable enables operable communication between a camera and the RPI. In particular embodiments, a USB connection enables operable communication between the RPI and the LUCID platform. In particular embodiments, an ethernet cable enables operable communication between the RPI and the LUCID platform. In particular embodiments, the RPI is in operable communication with the LUCID platform via a cloud network.

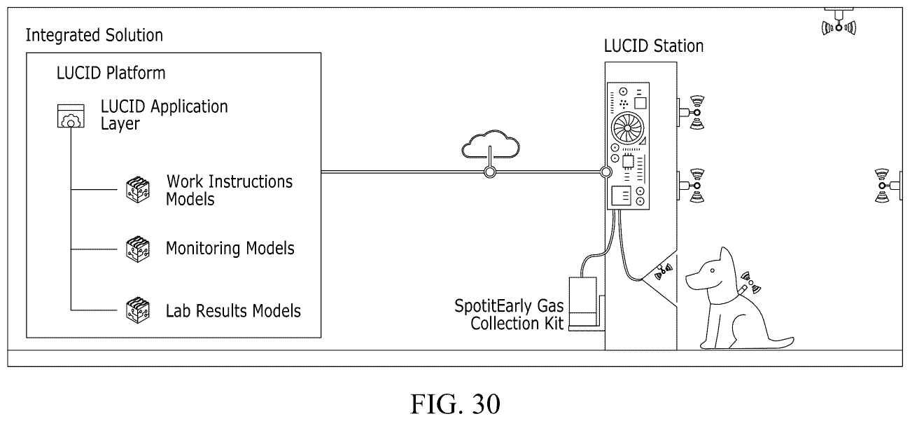

FIG. 30 depicts an example disease detection system. In particular embodiments, the disease detection comprises a LUCID platform and a LUCID station which are connected to each via the cloud. In particular embodiments, the LUCID platform comprises an application layer which comprises work instruction models, monitoring models, and lab results models. In particular embodiments, the LUCID platform comprises software which manages the test. For example, the LUCID platform controls the olfactometer system and instructs the olfactometer system on the sample type to be released (e.g., a test sample or a control sample), which samples to test, and when to release a sample. A control sample is a validated sample, and its positive or negative cancer state is known. The LUCID platform also instructs the lab worker on when to introduce a canine to the test room and the ID of the canine to bring. The LUCID platform also determines the order of canines which are to be used for testing. In particular embodiments, the LUCID platform determines a time at which a canine is awarded a treat while doing a test run. For example, if a first canine has already identified a test sample to be positive, and a second canine also identifies said test sample to be positive, then the second canine is awarded a treat. One benefit of rewarding the canine during a test run is to increase the focus and motivation of the canine so that it may examine a higher number of samples during a test run. In particular embodiments, a first canine examines the highest number of samples, a second canine examines the second-highest number of samples, a third canine examines the third-highest number of samples, and so on. Although this disclosure describes rewarding canines in a particular manger, this disclosure contemplates rewarding canines in any suitable manner.

In particular embodiments, the LUCID station comprises an olfactometer system. In particular embodiments, the LUCID station knows the headspace for the sample on the lab side, and pushes it accurately onto the test side of the olfactometer system so that a canine may sniff the sample. In particular embodiments, the LUCID station receives information from the patient's sample collection kit. For example, a patient may scan a QR code to launch a website on their computing device and submit patient data to the LUCID station via the website.

### 8. Machine-Learning Architecture

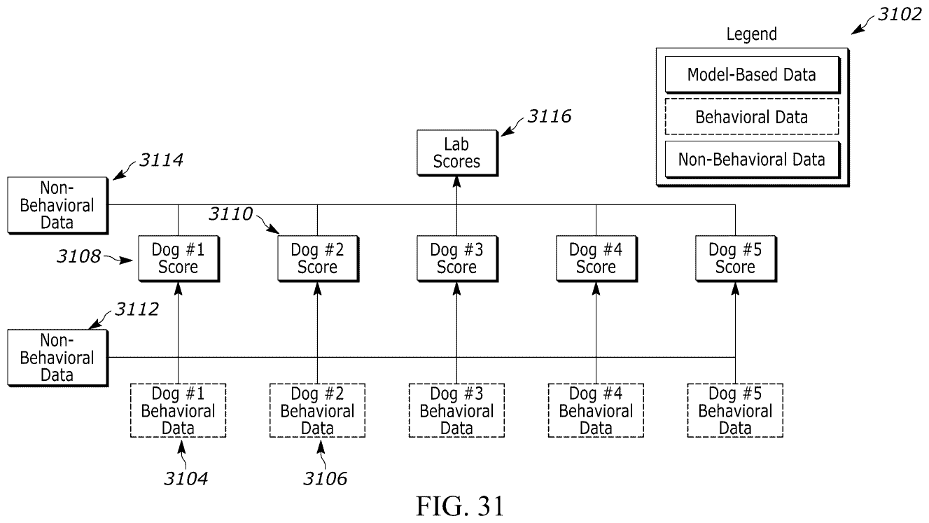

FIG. 31 illustrates an example of a model 3102 of the disease-detection system utilizing a stacked learning approach which is suitable for predicting a lab result. This architecture addresses the prediction problem in a hierarchical way, where a dog-specific predictive model is fitted for each detection animal, e.g. a dog, and then the output of the dog-specific predictive models is the input of the lab-result ML-model.

In particular embodiments, a ML-model is created for each detection animal. That is, there may be a plurality of ML-models, wherein a particular ML-model is associated with a particular animal. For example, first ML-model is fitted for Dog #1 and fitted ML-model is created for Dog #2 using relevant data (e.g. behavioral data and physiological) for each dog. Next, a lab-result ML-model is fitted for a pack of dogs (e.g., Dog #1, Dog #2, etc.), using the scores of the first ML-model, the second ML-model, etc., and non-behavioral data 3114.

As an example, and not by way of limitation, Dog #1 behavioral data 3104 is input into the first ML-model (created for Dog #1), and Dog #2 behavioral data 3106 is input into the second ML-model (created for Dog #2). This method repeats for the total number of dogs. That is, dog score 3108 is determined using the behavioral data 3104 for Dog #1 and non-behavioral data 3112, and dog score 3110 is determined using the behavioral data 3106 and non-behavioral data 3112 for Dog #2. The non-behavioral data 3112 may comprise one or more of the patient data (e.g. family medical history, patient medical history, patient age, patient gender, and patient demographic data), or environmental data described herein. This respective method is performed for each respective animal. Dog #1 Score is an initial confidence score associated with Dog #1, Dog #2 Score is an initial confidence score associated with Dog #2, etc.

In particular embodiments, the non-behavioral data 3112 and 3114 may comprise data from a previous test using the systems and method described herein performed on the patient. For example, a patient undergoing cancer treatment may have a first biological sample tested using the disease-detection system, and after a period of time, have a second biological sample tested using the disease-detection system. In particular embodiments, data from prior tests on the first biological sample is already stored by the disease-detection system when testing the second biological sample. In particular responses, the ML-model compares sensor and inputted data associated with the first biological sample, with sensor and inputted data associated with the second biological sample, when making a determination on a disease state and a confidence score.

Next, the fitted dog scores (e.g., 3108 and 3110) are aggregated by a lab-result ML-model, which also receives non-behavioral data 3114 as an input, to determine a lab score 3116. The non-behavioral data 3114 may comprise one or more of the patient data (e.g., family medical history, patient medical history, patient age, patient gender, and patient demographic data) and environmental data described herein. In particular embodiments, lab score 3116 is calculated based on a probability of the disease state. In particular embodiments, lab score 3116 is calculated based on a probability of the disease state and a confidence prediction interval.

Although this disclosure describes and illustrates an example ML-model of the disease-detection system utilizing a stacked learning approach comprising a plurality of steps, this disclosure contemplates any suitable ML-model for disease-detection including any suitable steps, which may include all, some, or none of the steps of the method of FIG. 31.

In particular embodiments, a model may be developed for a plurality of dogs. For example, a model may be developed for a class of dogs. A particular class of dogs may be grouped by dogs of a similar breed, dogs which have a similar sniffing duration, or other behavioral characteristics of the dogs. Each class of dogs undergoes a similar training process. The LUCID platform may also re-evaluate whether each dog is classified in the correct class and adjust the classification as needed.

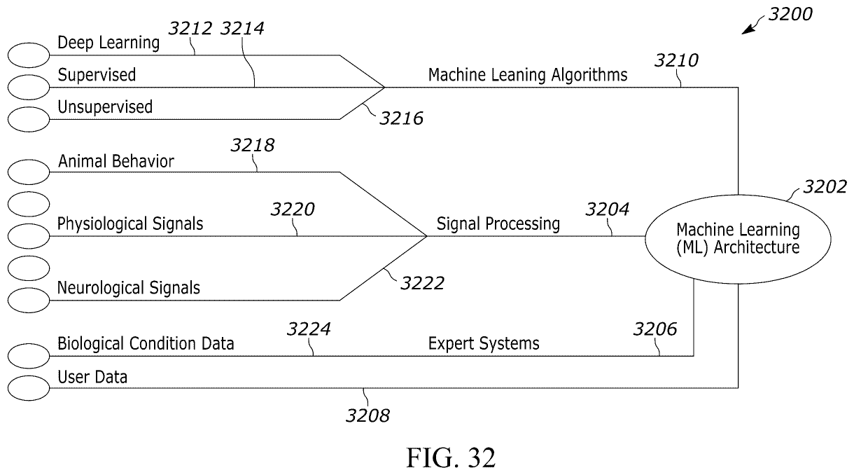

FIG. 32 illustrates a diagram 3200 of an example ML architecture 3202 that may be utilized in a disease-detection system using detection animals, in accordance with the presently disclosed embodiments. In particular embodiments, the ML architecture 3202 may be implemented utilizing, for example, one or more processing devices that may include hardware, e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), and/or other processing device(s) that may be suitable for processing various data and making one or more decisions based thereon), software (e.g., instructions running/executing on one or more processing devices), firmware (e.g., microcode), or some combination thereof.

In particular embodiments, as depicted by FIG. 32, the ML architecture 3202 may include signal processing algorithms and functions 3204, expert systems 3206, and user data 3208. In particular embodiments, the ML algorithms and functions 3210 may include any statistics-based algorithms that may be suitable for finding patterns across large amounts of data. As an example, and not by way of limitation, in particular embodiments, the ML algorithms and functions 3210 may include deep learning algorithms 3212, supervised learning algorithms 3214, and unsupervised learning algorithms 3216.

In particular embodiments, the deep learning algorithms 3212 may include any artificial neural networks (ANNs) that may be utilized to learn deep levels of representations and abstractions from large amounts of data. As an example, and not by way of limitation, the deep learning algorithms 3212 may include ANNs, such as a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), deep reinforcement learning, and so forth.

In particular embodiments, the supervised learning algorithms 3214 may include any algorithms that may be utilized to apply, for example, what has been learned in the past to new data using labeled examples for predicting future events. As an example, and not by way of limitation, starting from the analysis of a known training dataset, the supervised learning algorithms 3214 may produce an inferred function to make predictions about the output values. The supervised learning algorithms 3214 can also compare its output with the correct and intended output and find errors in order to modify the supervised learning algorithms 3214 accordingly. On the other hand, the unsupervised learning algorithms 3216 may include any algorithms that may be applied, for example, when the data used to train the unsupervised learning algorithms 3216 are neither classified nor labeled. As an example, and not by way of limitation, the unsupervised learning algorithms 3216 may study and analyze how systems may infer a function to describe a hidden structure from unlabeled data.

In particular embodiments, the signal processing algorithms and functions 3204 may include any algorithms or functions that may be suitable for automatically manipulating signals, including animal behavior signals 3218, physiological signals 3220, and neurological signals 3222 (e.g., EEG, fNIR, fMRI, or MRI signals).

In particular embodiments, the expert systems 3208 may include any algorithms or functions that may be suitable for recognizing and translating signals from detection animals and user data 3226 into biological condition data 3224. Examples of ML planning may include AI planning (e.g. classical planning, reduction to other problems, temporal planning, probabilistic planning, preference-based planning, or conditional planning.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### 8.1. Machine-Learning Overview

In particular embodiments, the disease-detection system comprises a plurality of ML-models. Features of the ML-model are based on one or more behavioral events (e.g., sniffing and sitting events), physiological events, neurological events in testing, or patient data. Example behavioral, physiological, and neurological events are described herein. In particular embodiments, a custom ML-model is created for each detection animal. As an example, and not by way of limitation, a custom ML-model is created to analyze the behavior, physiological response, or neurological response of the detection animal during a test run. As another example, the system comprises an ML-model which calculates a dog score based on behavioral and non-behavioral inputs. As another example, the system comprises an ML-model which analyzes the physiological data from a detection animal. As another example, the system comprises an ML-model which may use data from the sensors described herein to calculate a measurement of indecisiveness in the detection animal. As another example, the system comprises a ML-model customized to monitor a behavioral drift (e.g., a behavioral abnormality) of a detection animal. As another example, the system comprises a neurological-based ML-model which analyzes a brain signal from a detection animal. As another example, the system comprises a neurological-based ML-model which predicts a disease state. As another example, the system comprises a neurological-based ML-model which predicts a cancer type. As another example, the system comprises a neurological-based ML-model which predicts a cancer stage. As another example, the system comprises a neurological-based ML-model for verification of a cancer state. As another example, the system comprises a custom ML-model created for a pack of detection animals. In particular embodiments, the disease-detection system stores one or more black box features to be used in the one or more ML-models. In particular embodiments, the ML-based disease-detection model generates feature representations based on one or more of the behavioral, physiological, neurological data, or patient data.

The aggregations are calculated in multiple aggregative levels. The following list describes example aggregations for dog-round per a specific biological sample. Below, ‘X’ denotes the dog name, and ‘y’ the round name:
X={Pluto, Mars, . . . }
y={main1,main1+main2, cleaning, lab_manager, suspicious}

### Features:

1. y_n_X (number of sniffs of dog X at round y)
2. y_sit_X (number of sits of dog X sits at round y)
3. y_sit_prop_X (proportion of sits out of sniffs of dog X at round y)
4. y_multiple_sniffs_sit_n_X (the number of runs with sitting for X,y)
5. y_multiple_sniffs_sit_avg_X (the average number of sniffs per runs with sitting for X,y)
6. y_multiple_sniffs_sit_max_X (the maximum number of sniffs for sitting runs for X,y)
7. y_multiple_sniffs_nosit_n_X, y_multiple_sniffs_nosit_avg_X,
y_multiple_sniffs_nosit_max_X (same as above, but for runs without sitting).
8. y_sniff_duration_sit_avg_X, y_sniff_duration_sit_max_X (the average and the
maximum duration of a sniff for sniffs with sitting)
9. y_sniff_duration_nosit_avg_X, y_sniff_duration_nosit_max_X (the average and the
maximal duration of a sniff for sniffs without sitting)
10. y_sniff2sit_duration_avg_X (average duration between stop sniff to start sit for X,y)
12. y_sniff2sit_duration_max_X (maximal duration between stop sniff to start sit for X,y)
   Additional features used in the ML-model are:
12. main1valid_X (indicator for valid main round for dog X)
13. y_is_suspicous (indicator whether the sample was suspicious at round y)
14. lab_result_X
15. Lab_result
16. lab_result_Canine Team Rule
17. Lab_result_Canine Team Rule
   Model's output:

The ML-model output contains two files:
1. Cancer probability (a scalar between 0 to 1)
2. Predicted confidence interval (a range between 0-1)

### 8.2. Training Overview

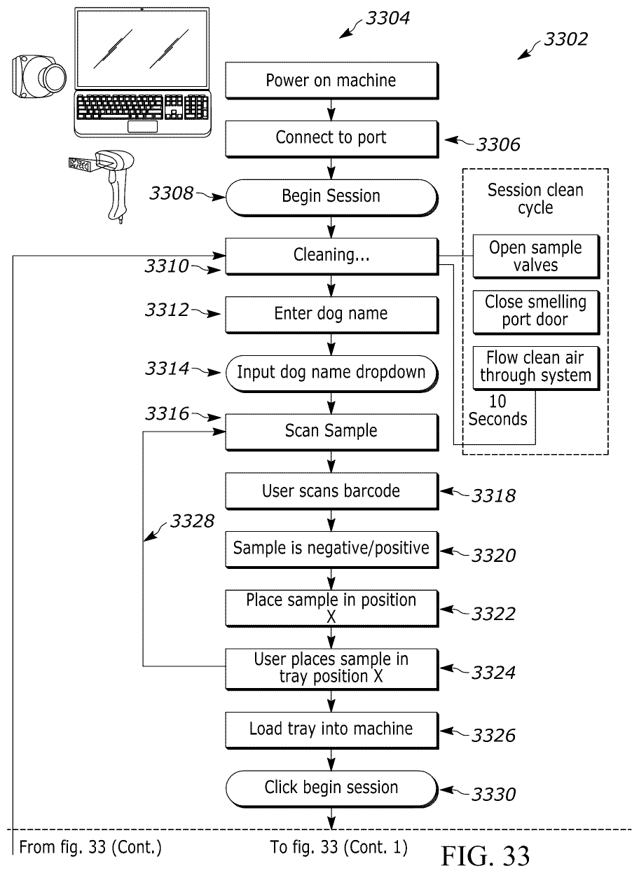

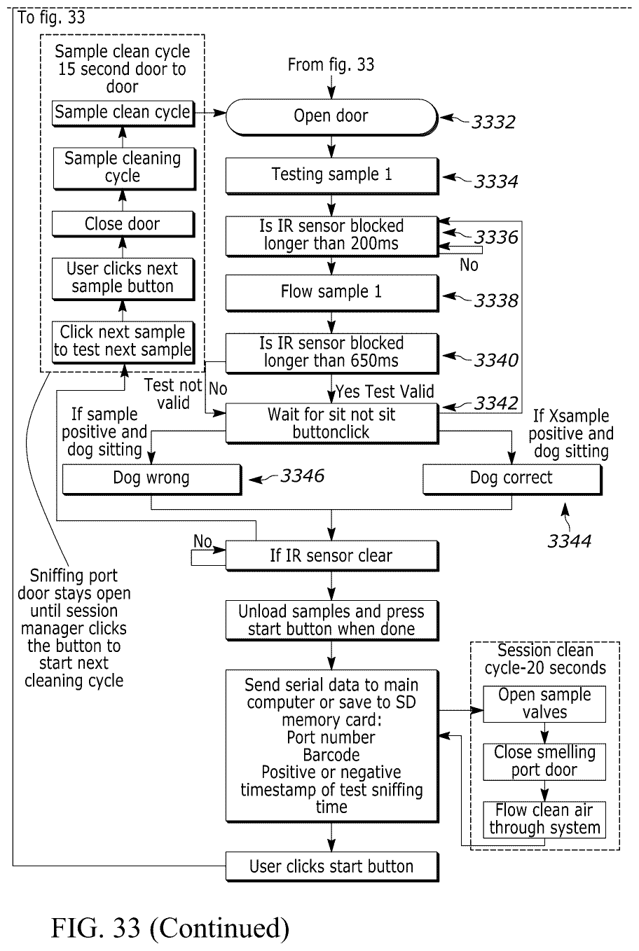

FIG. 33 depicts an example method 3302 for training the ML-based disease-detection model using an olfactometer system. In particular embodiments, the model may be trained in a plurality of aspects, including test management, performance monitoring, and analytics which support training plans.

The method begins at step 3304 wherein the machine is turned on. Then the system connects to a plurality of sample ports at step 3306, and begins a session at step 3308. At step 3310, a clean process is performed to clean the step. An example cleaning procedure for cleaning an olfactometer system is described herein. The cleaning procedure comprises opening the sample valves, closing the sampling port door, and flowing clean air through the system for a predetermined amount of time (e.g., 10 seconds). At steps 3312 and 3314, a particular detection animal is identified to the model. The identifying information may comprise a name of the detection animal. Next, at step 3316, the user receives an instruction to scan a biological sample for testing, and at step 3318 the user scans the biological sample. Next, the operator (e.g., a lab manager) inputs into the model in indication of whether the sample (e.g. a training sample) is positive or negative for cancer at step 3320. Then, at steps 3322-3326, the sample is placed into position, step 3322 comprising placing a sample in position, step 3324 comprising placing the sample in tray position X, and step 3326 comprising loading the tray into the machine. In particular embodiments, the position may be at a particular receptacle in an olfactometer system. In other embodiments, the position may be proximate to a sampling port. In particular embodiments, the sample is loaded onto a tray. In certain embodiments, if a user improperly places the sample into position, the system alerts the user and instructs the user to re-perform steps 3316-3324 to properly load the sample into position. Next, at step 3330, a user selects an input which initializes a section. The next steps are depicted on FIG. 33 (Cont.).

After the session has begun, the door to the sampling port opens at step 3332. Next, at step 3334, the system provides an indicator that testing is active. At step 3336, the system receives data from one or more IR sensors of the sampling port. The IR sensor measures the length of time a detection animal performs a sniff. In particular embodiments, a sniff of 200 ms constitutes a valid sniff. Upon a determination of a valid sniff of 200 ms or more, the method proceeds to step 3338 wherein a sample is exposed to the detection animal through a flow path. Upon a determination that a sniff was less than 200 ms, the system repeats step 3336 and waits for a new sniff from a detection animal. The system continues to receive data from the IR sensor. At step 3340, the system receives data on whether the IR sensor is blocked for longer than 650 ms. In particular embodiments, if the IR sensor is not blocked for 650 ms, then the sniff is not considered valid. In particular embodiments, if an IR sensor is blocked for 650 ms or more, then the test is considered valid.

At step 3342, the system receives an operator input on whether the detection animal sits. A body pose of a sitting position indicates the presence of cancer in a biological sample. A body pose comprising a standing position indicates that cancer was not detected in the biological sample. The disease state of the training sample (e.g., a biological sample) is known by the lab operator. A user or a machine may input the body pose position of the detection animal so that the ML-based disease-detection model receives information on whether the detection animal correctly identified the sample. If the detection animal makes a correct determination on the state of the sample, then the system provides an indication 3344 that the dog was correct. If the detection animal makes an incorrect determination on the state of the sample, then the system provides an indication 3346 that the dog was wrong. Next, the result, comprising either a dog correct indication 3344 or dog wrong indication 3346, is logged by the system.

Next, at step 3348, the system determines whether the IR sensor detects any obstruction. If the IR sensor is clear, then the system outputs an alert instructing a user to unload the samples. Next, data associated with the test, including the port number, bar code of the sample, a positive or negative detection event, the time of the test, and the sniffing time, are saved in the system. Next, the system may optionally perform a cleaning cycle.

### 8.3. Working Protocol

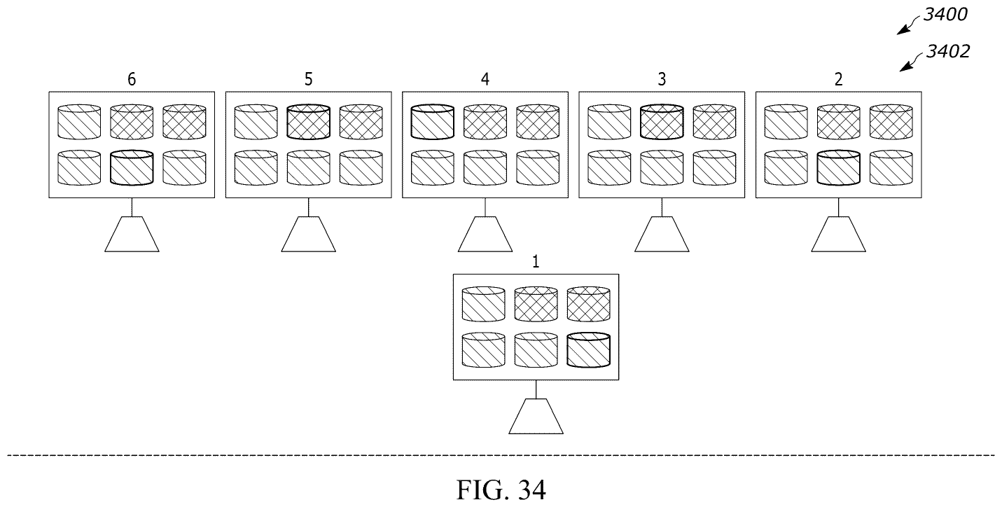

FIG. 34 illustrates an example working protocol 3400. In particular embodiments, an automated algorithm manages the presentation of the sample during the round, achieving an optimized canine performance and scale. 3402 depicts the protocol during a first run, or a second run. Each run comprises having a canine sniff one or more sampling ports connected to the olfactometer system. In the example depicted in FIG. 34, there are six sampling ports, and each sampling port is connected to six receptacles, each receptacle capable of holding a sample. 3402 depicts six receptacles connected to a sampling port—each receptacle contains either a test sample or a service sample. 3402 depicts the different sample types using two different patterns: a striped pattern and a crisscross pattern. In particular embodiments, a service sample may be a sample that the LUCID platform estimates to be a particular state. For example, the LUCID platform may place a sample that it estimates to be likely positive in a test pool comprising likely negative samples. In particular embodiments, a service sample has a positive or negative state that is known by the LUCID platform. In particular embodiments, the canine may be provided with a reward when it correctly identifies the status of a service sample. In particular embodiments, the canine is provided with a treat as the reward, and the treat is a food item for the canine. At first run 3402, a canine is exposed to one or more samples from one or more sampling ports. The particular sample to be exposed is determined by the LUCID platform. The LUCID platform determines the sample(s) to expose to the canine—for example, the LUCID platform may expose the same sample during the second run. Alternatively, the LUCID platform may expose a different sample during the second run. The LUCID platform has the flexibility to select particular samples for optimizing performance and scale. The sample that is exposed via the sampling port is outlined in black in FIG. 34. The test protocol enables a large number of samples to be loaded for testing and also enables exposing different samples in a sampling round. Although the figure depicts one sample being exposed via one sampling port at a time, in particular embodiments, multiple samples may be exposed at a time via one sampling port.

### 9. Exemplary Methods

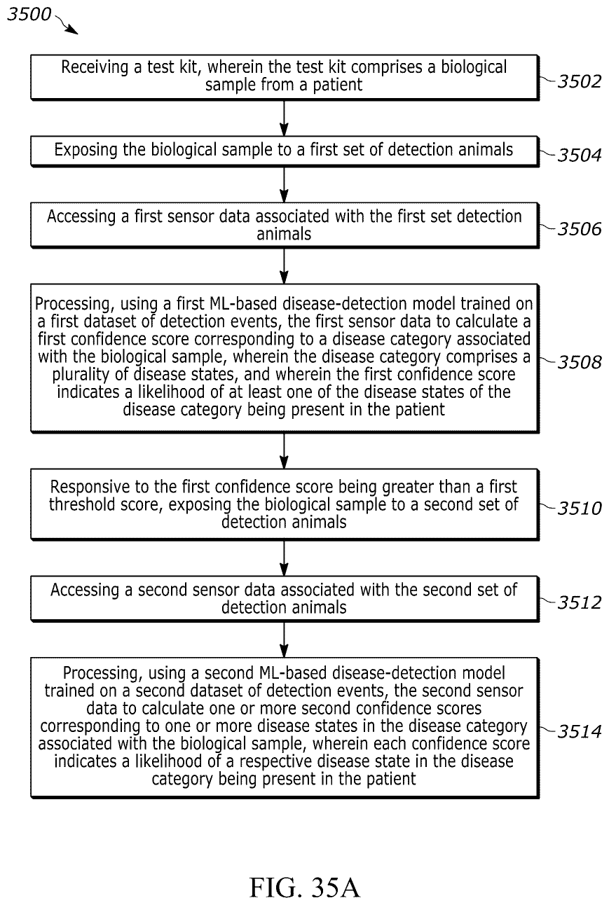

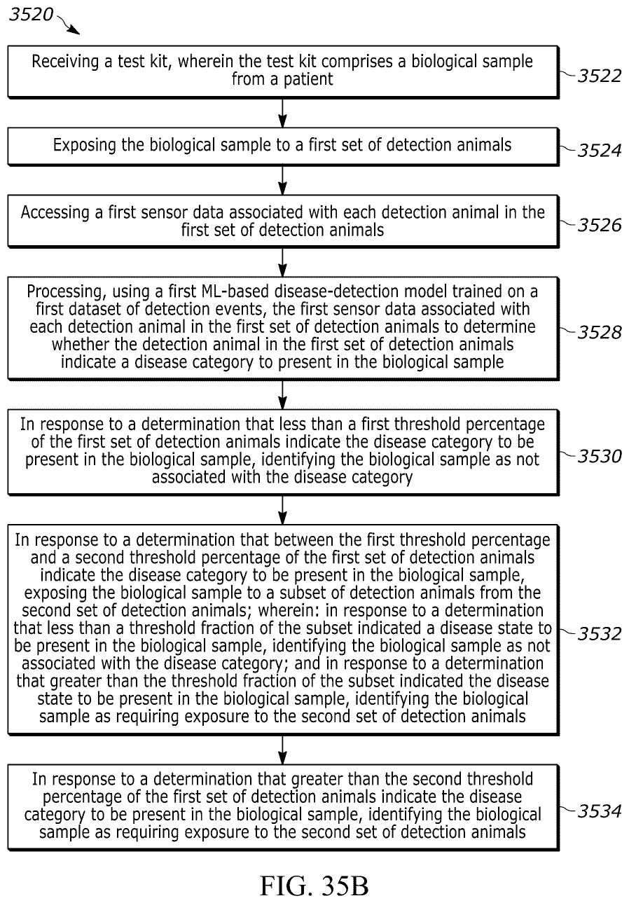

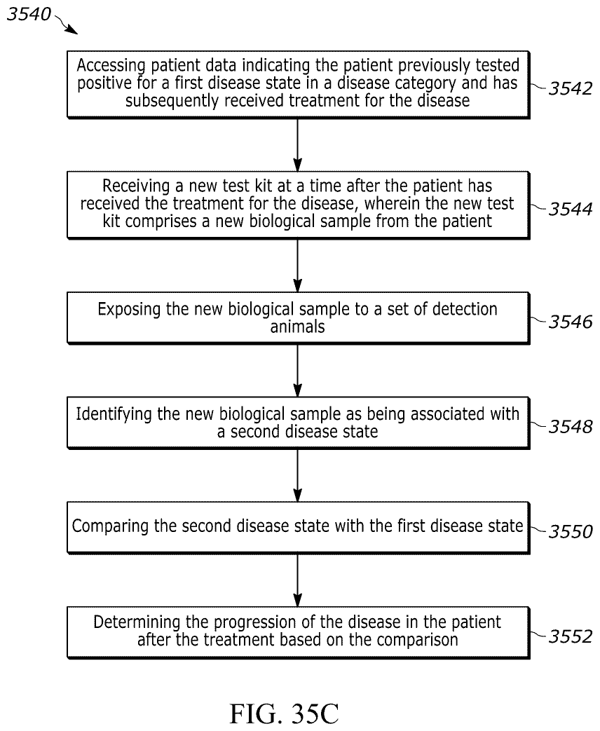

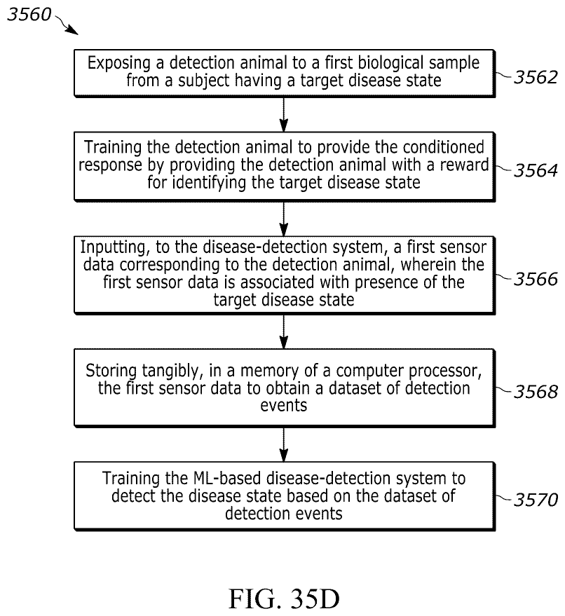

FIG. 35A illustrates an example method 3500 for disease detection. The method may begin at step 3502, where the method may comprise receiving a test kit, wherein the test kit comprises a biological sample from a patient. At step 3504, the method may comprise exposing the biological sample to a first set of detection animals. At step 3506, the method may comprise accessing a first sensor data associated with the first set detection animals. At step 3508, the method may comprise processing, using a first ML-based disease-detection model trained on a first dataset of detection events, the first sensor data to calculate a first confidence score corresponding to a disease category associated with the biological sample, wherein the disease category comprises a plurality of disease states, and wherein the first confidence score indicates a likelihood of at least one of the disease states of the disease category being present in the patient. At step 3510, the method may comprise responsive to the first confidence score being greater than a first threshold score, exposing the biological sample to a second set of detection animals. At step 3512, the method may comprise accessing a second sensor data associated with the second set of detection animals. At step 3514, the method may comprise processing, using a second ML-based disease-detection model trained on a second dataset of detection events, the second sensor data to calculate one or more second confidence scores corresponding to one or more disease states in the disease category associated with the biological sample, wherein each confidence score indicates a likelihood of a respective disease state in the disease category being present in the patient. Although this disclosure describes and illustrates particular steps of the method of FIG. 35A as occurring in a particular order, this disclosure contemplates any suitable steps of the method of FIG. 35A occurring in any suitable order. Moreover, although this disclosure describes and illustrates an example method for disease detection including the particular steps of the method of FIG. 35A, this disclosure contemplates any suitable method for monitoring the clinical status of a disease or disorder in a subject including any suitable steps, which may include all, some, or none of the steps of the method of FIG. 35A, where appropriate.

FIG. 35B illustrates another example method 3520 for disease detection. The method may begin at step 3522, where the method may comprise receiving a test kit, wherein the test kit comprises a biological sample from a patient. At step 3524, the method may comprise exposing the biological sample to a first set of detection animals. At step 3526, the method may comprise accessing a first sensor data associated with each detection animal in the first set of detection animals. At step 3528, the method may comprise processing, using a first ML-based disease-detection model trained on a first dataset of detection events, the first sensor data associated with each detection animal in the first set of detection animals to determine whether the detection animal in the first set of detection animals indicate a disease category to present in the biological sample. At step 3530, the method may comprise, in response to a determination that less than a first threshold percentage of the first set of detection animals indicate the disease category to be present in the biological sample, identifying the biological sample as not associated with the disease category. At step 3532, the method may comprise in response to a determination that between the first threshold percentage and a second threshold percentage of the first set of detection animals indicate the disease category to be present in the biological sample, exposing the biological sample to a subset of detection animals from the second set of detection animals; wherein: in response to a determination that less than a threshold fraction of the subset indicated a disease state to be present in the biological sample, identifying the biological sample as not associated with the disease category; and in response to a determination that greater than the threshold fraction of the subset indicated the disease state to be present in the biological sample, identifying the biological sample as requiring exposure to the second set of detection animals. At step 3534, the method may comprise in response to a determination that greater than the second threshold percentage of the first set of detection animals indicate the disease category to be present in the biological sample, identifying the biological sample as requiring exposure to the second set of detection animals. Although this disclosure describes and illustrates particular steps of the method of FIG. 35B as occurring in a particular order, this disclosure contemplates any suitable steps of the method of FIG. 35B occurring in any suitable order. Moreover, although this disclosure describes and illustrates an example method for disease detection including the particular steps of the method of FIG. 35B, this disclosure contemplates any suitable method for monitoring the clinical status of a disease or disorder in a subject including any suitable steps, which may include all, some, or none of the steps of the method of FIG. 35B, where appropriate.

FIG. 35C illustrates an example method 3540 for determining a progression of a disease in a patient undergoing a treatment. The method may begin at step 3542, where the method may comprise accessing patient data indicating the patient previously tested positive for a first disease state in a disease category and has subsequently received treatment for the disease. At step 3544, the method may comprise receiving a new test kit at a time after the patient has received the treatment for the disease, wherein the new test kit comprises a new biological sample from the patient. At step 3546, the method may comprise exposing the new biological sample to a set of detection animals. At step 3548, the method may comprise identifying the new biological sample as being associated with a second disease state. At step 3550, the method may comprise comparing the second disease state with the first disease state. At step 3552, the method may comprise determining the progression of the disease in the patient after the treatment based on the comparing. Although this disclosure describes and illustrates particular steps of the method of FIG. 35C as occurring in a particular order, this disclosure contemplates any suitable steps of the method of FIG. 35C occurring in any suitable order. Moreover, although this disclosure describes and illustrates an example method for determining a progression of a disease in a patient undergoing a treatment, including the particular steps of the method of FIG. 35C, this disclosure contemplates any suitable method for monitoring the clinical status of a disease or disorder in a subject including any suitable steps, which may include all, some, or none of the steps of the method of FIG. 35C, where appropriate.

FIG. 35D illustrates an example method 3560 for training a detection animal to provide a conditioned response to be used with a machine learning-based (ML-based) disease-detection system. The method may begin at step 3562, where the method may comprise exposing a detection animal to a first biological sample from a subject having a target disease state. At step 3564, the method may comprise training the detection animal to provide the conditioned response by providing the detection animal with a reward for identifying the target disease state. At step 3566, the method may comprise inputting, to the disease-detection system, a first sensor data corresponding to the detection animal, wherein the first sensor data is associated with presence of the target disease state. At step 3568, the method may comprise storing tangibly, in a memory of a computer processor, the first sensor data to obtain a dataset of detection events. At step 3570, the method may comprise training the ML-based disease-detection system to detect the disease state based on the dataset of detection events. Although this disclosure describes and illustrates particular steps of the method of FIG. 35D as occurring in a particular order, this disclosure contemplates any suitable steps of the method of FIG. 35D occurring in any suitable order. Moreover, although this disclosure describes and illustrates an example method for training a detection animal to provide a conditioned response to be used with a machine learning-based (ML-based) disease-detection system, including the particular steps of the method of FIG. 35D, this disclosure contemplates any suitable method for monitoring the clinical status of a disease or disorder in a subject including any suitable steps, which may include all, some, or none of the steps of the method of FIG. 35D, where appropriate.

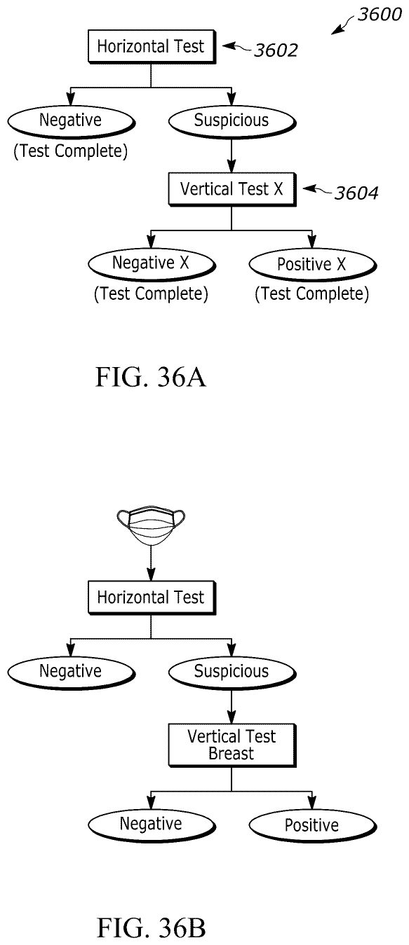

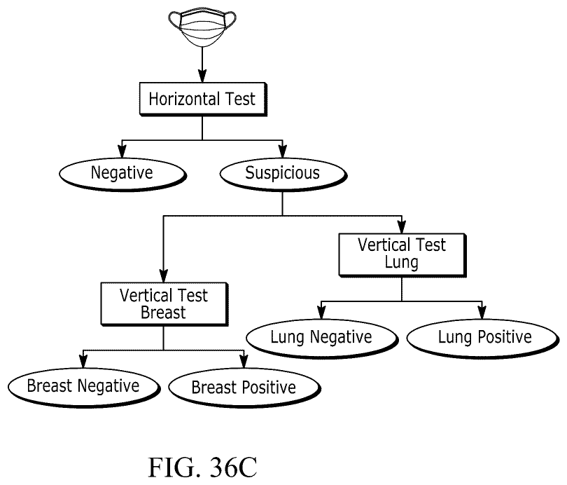

FIG. 36A illustrates an example test protocol. Horizontal test 3202 refers to a first stage of testing wherein samples are either flagged as “negative” or “suspicious.” “Negative” refers to a non-cancerous determination and “suspicious” refers to a potentially positive determination. Samples that are flagged by the LUCID system as “suspicious” are sent for a second stage of testing, indicated by “vertical X” 3604, where X denotes any cancer type. At stage vertical X 3604, samples undergo one or more test modules. For example, a first vertical X module may be to test a sample for a first type of cancer (e.g., breast cancer). The first vertical X module has its own set of canines for that particular module. Further, a second vertical X module may be to test a sample for a second type of cancer (e.g., lung cancer). The second vertical X module has its own set of canines for that particular module. Thus, although the figure depicts one vertical X module, a plurality of vertical X modules may exist on the platform. The vertical X module(s) 3604 are more conservative. As an example, and not by way of limitation, the LUCID platform is more conservative in the vertical X module(s) when identifying positive samples. Although the figure describes a test protocol in a particular manner, a test protocol may be run in any suitable manner. With reference to FIG. 36A, FIG. 36B illustrates a specific example test protocol for identifying breast cancer. With reference to FIGS. 36A and 36B, FIG. 36C illustrates a specific example test protocol for identifying breast cancer or lung cancer.

In particular embodiments, the LUCID platform has a target of one positive sample per run. Although the status of test samples is not known during a double-blind run, the LUCID platform may place one likely-positive sample among many likely-negative or known-negative samples. In other words, the LUCID platform estimates a percent of positive samples and adds in a number of known-negative or likely-negative samples to achieve its desired ratio of positive to negative samples in a test run. For example, the LUCID platform may combine samples which are highly likely to be positive with samples that have a low likelihood of being positive. In other words, the LUCID platform may control the number of expected positive samples that a detection animal is exposed to in a test run.

In particular embodiments, the LUCID platform randomizes a plurality of components to eliminate bias. In particular embodiments, the LUCID platform maximizes the number of samples in a run without positive samples. In particular embodiments, the LUCID platform utilizes a rewarding mechanism to improve the performance and scale of the test. In particular embodiments, the LUCID platform restricts having runs with only a minimal number of samples.

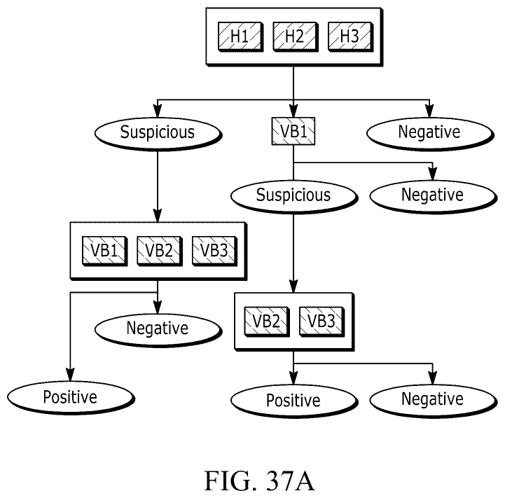

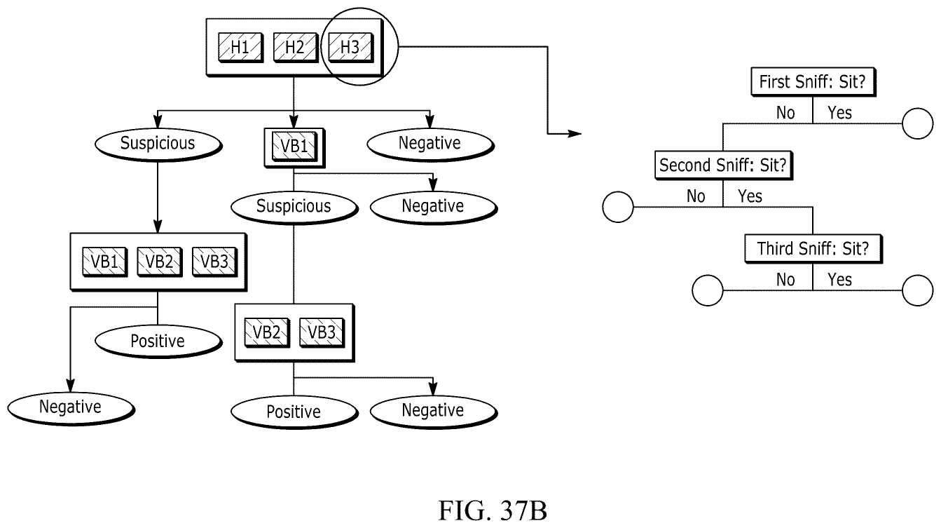

FIG. 37A illustrates an exemplary machine-learning (ML) architecture comprising the first stage of testing (horizontal test) using a first set of detection animals (e.g., dogs H1, H2 and H3) for identifying presence of a disease category (e.g., cancer) in a biological sample. In particular embodiments, the LUCID system calculates based on the first sensor data, a first confidence score corresponding to a disease category associated with the biological sample, wherein the confidence score indicates a likelihood of at least one of the disease states of the disease category being present in the patient.

Samples are flagged by the LUCID system as “negative” (absence of the disease category) or “suspicious” (potentially positive for the disease category) In particular embodiments, the LUCID system flags the biological sample as negative when 0% to 9% of the first set of dogs exposed to the biological sample provide a positive response. A positive response indicates the dog detected the disease category. In particular embodiments, the LUCID system flags the biological sample as suspicious when 40% to 100% of the first set of dogs exposed to the biological sample provide a positive response.

In particular embodiments, the LUCID system flags the biological sample as suspicious for the disease category (e.g., cancer) when 40% to 100% of the first set of dogs exposed to the biological sample provide a positive response, and the sample advances to a second stage of testing (vertical test) to identify the disease state (e.g., breast cancer) using a second set of dogs (e.g., VB1, VB2 and VB3).

In particular embodiments, in the vertical test, the LUCID system flags the biological sample as negative when 0% to 9% of the second set of dogs exposed to the biological sample provide a positive response for the disease state (e.g., breast cancer). In particular embodiments, the LUCID system flags the biological sample as suspicious when 40% to 100% of the second set of dogs exposed to the biological sample provide a positive response for the disease state after exposure to the biological sample.

In particular embodiments, when 10-39% of the first set of dogs exposed to the biological sample provide a positive response, one or more dogs from a second set of dogs is exposed to the biological sample and based on the response from the second set of dogs, the sample is flagged by the LUCID system as “negative’ or “suspicious.” Samples identified as suspicious advance to the second stage of testing (vertical test) as described above.

FIG. 37B illustrates an exemplary machine-learning (ML) architecture for analyzing an example dynamic flow between sniffs (e.g., 1-3 sniffs) taken by the dog when exposed to a breath sample. FIG. 37B illustrates an example wherein the sample is determined to be negative for the disease category when the dog identifies the sample to be negative after two sniffs. For example, when a dog does not sit after a first sniff (indicating an absence of the disease state), the dog is exposed to the same sample for a second sniff. If the dog does not sit after the second sniff, then the dog is no longer exposed to that particular sample. If the dog sits during the second sniff, then the same or different dog is exposed to the sample for a third sniff. Although this patent describes an iterative process of sample exposure to a detection animal in a particular manner, this disclosure contemplates any suitable iterative process for sample exposure to a detection animal.

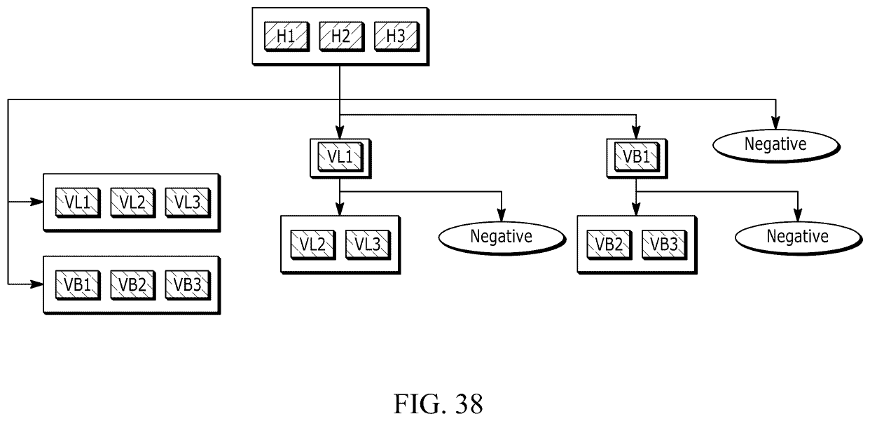

With reference to FIGS. 37A and 38 illustrate an example machine-learning (ML) architecture for identifying a disease category and example disease states breast cancer (VB) and lung cancer (VL). The ML architecture comprises a first stage of testing (horizontal test) using a first set of dogs (e.g., H1, H2 and H3) for identifying presence of a disease category followed by identifying presence of a specific disease state within the identified disease category using the LUCID system as described above.

In particular embodiments, when a particular subset of the first set of dogs exposed to the biological sample provide a positive response, the sample advances to a second stage of testing (vertical test) using a second set of dogs specific for a disease state (e.g., VB1 for breast cancer detection, VL1 for lung cancer detection). In particular embodiments, the particular subset corresponds to a percentage of dogs that provided a positive response, e.g., a percentage selected from a range of between 33% and 66%. In this instance, if a positive response is provided by the second set of dogs, the sample advances to additional second set of dogs specific for the disease state (e.g., VL2, VL3 for lung cancer) to confirm that the disease state is indeed lung cancer.

### 9.1. ML Model Performance

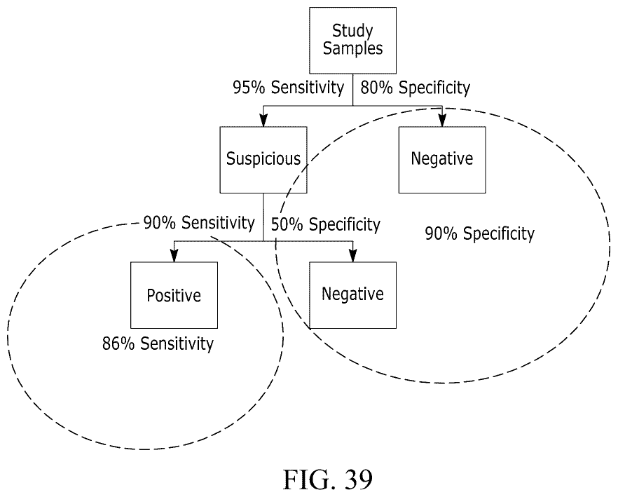

FIG. 39 illustrates exemplary performance characteristics for horizontal and vertical tests.

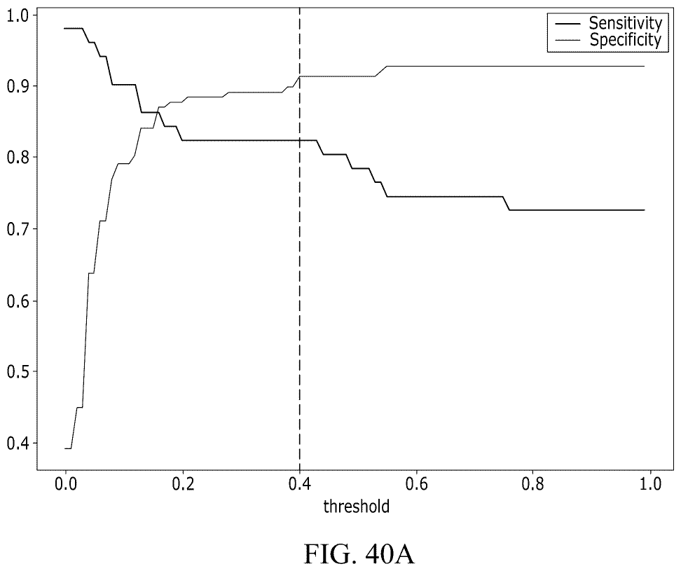

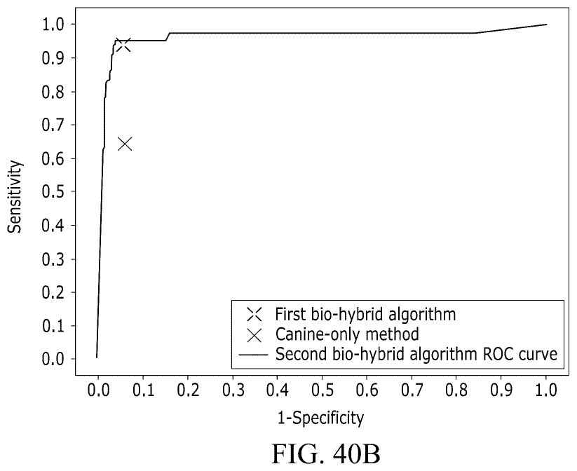

FIGS. 40A-40B illustrate performance of the predictive model, wherein the predictive model utilized a test procedure comprising four rounds: a “main round” which tested all samples, a “cleaning round” which focused on testing samples classified as negative in the main round, a “suspicious round” which was applicable in specific scenarios, and a “lab manager round” which was employed in certain scenarios. Improving the models for the main round (e.g., the round which tests all samples) has resulted in a 9.8% increase in sensitivity compared to an alternative scoring system (non-ML-based). Further, refining the cleaning round (e.g., the round which focuses on samples classified as negative in the main round) has resulted in a 7% increase in sensitivity without compromising specificity. FIG. 40A illustrates that a decrease in the threshold corresponds to an increase in sensitivity but a decrease in specificity, and vice versa. The threshold is used for classification and may be set to different values depending on the desired sensitivity and specificity. Each threshold defines different properties of the trained model in terms of expected sensitivity and specificity.

FIG. 40B illustrates an example ROC curve for a second bio-hybrid algorithm. The curve's trajectory demonstrates the tradeoff between sensitivity and specificity resulting from adjusting the model's threshold. The area under the curve (AUC) of the second bio-hybrid algorithm is 98.2%. In comparison, markers for the first bio-hybrid algorithm and the canine-only method lie below the ROC curve, suggesting a relatively lower prediction accuracy.

### 10. Computer System Overview

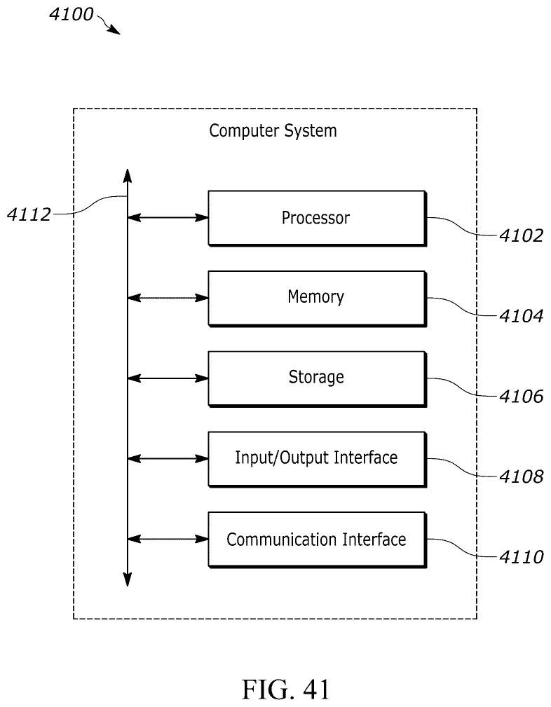

FIG. 41 illustrates an example computer system 4100 that may be utilized to perform a ML-based disease-detection method using detection animals in accordance with the presently disclosed embodiments. In particular embodiments, one or more computer systems 4100 perform one or more steps of one or more methods described or illustrated herein. In particular embodiments, one or more computer systems 4100 provide functionality described or illustrated herein. In particular embodiments, software running on one or more computer systems 4100 performs one or more steps of one or more methods described or illustrated herein or provides functionality described or illustrated herein. Particular embodiments include one or more portions of one or more computer systems 4100. Herein, reference to a computer system may encompass a computing device, and vice versa, where appropriate. Moreover, reference to a computer system may encompass one or more computer systems, where appropriate.

This disclosure contemplates any suitable number of computer systems 4100. This disclosure contemplates computer system 4100 taking any suitable physical form. As example and not by way of limitation, computer system 4100 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 4100 may include one or more computer systems 4100; be unitary or distributed; span multiple locations; span multiple machines; span multiple data centers; or reside in a cloud, which may include one or more cloud components in one or more networks.

Where appropriate, one or more computer systems 4100 may perform without substantial spatial or temporal limitation one or more steps of one or more methods described or illustrated herein. As an example, and not by way of limitation, one or more computer systems 4100 may perform in real time or in batch mode one or more steps of one or more methods described or illustrated herein. One or more computer systems 4100 may perform at different times or at different locations one or more steps of one or more methods described or illustrated herein, where appropriate.

In particular embodiments, computer system 4100 includes a processor 4102, memory 4104, storage 4106, an input/output (I/O) interface 4108, a communication interface 4110, and a bus 4112. Although this disclosure describes and illustrates a particular computer system having a particular number of particular components in a particular arrangement, this disclosure contemplates any suitable computer system having any suitable number of any suitable components in any suitable arrangement. In particular embodiments, processor 4102 includes hardware for executing instructions, such as those making up a computer program. As an example, and not by way of limitation, to execute instructions, processor 4102 may retrieve (or fetch) the instructions from an internal register, an internal cache, memory 4104, or storage 4106; decode and execute them; and then write one or more results to an internal register, an internal cache, memory 4104, or storage 4106. In particular embodiments, processor 4102 may include one or more internal caches for data, instructions, or addresses. This disclosure contemplates processor 4102 including any suitable number of any suitable internal caches, where appropriate. As an example, and not by way of limitation, processor 4102 may include one or more instruction caches, one or more data caches, and one or more translation lookaside buffers (TLBs). Instructions in the instruction caches may be copies of instructions in memory 4104 or storage 4106, and the instruction caches may speed up retrieval of those instructions by processor 4102.

Data in the data caches may be copies of data in memory 4104 or storage 4106 for instructions executing at processor 4102 to operate on; the results of previous instructions executed at processor 4102 for access by subsequent instructions executing at processor 4102 or for writing to memory 4104 or storage 4106; or other suitable data. The data caches may speed up read or write operations by processor 4102. The TLBs may speed up virtual-address translation for processor 4102. In particular embodiments, processor 4102 may include one or more internal registers for data, instructions, or addresses. This disclosure contemplates processor 4102 including any suitable number of any suitable internal registers, where appropriate. Where appropriate, processor 4102 may include one or more arithmetic logic units (ALUs); be a multi-core processor; or include one or more processors 4102. Although this disclosure describes and illustrates a particular processor, this disclosure contemplates any suitable processor.

In particular embodiments, memory 4104 includes main memory for storing instructions for processor 4102 to execute or data for processor 4102 to operate on. As an example, and not by way of limitation, computer system 4100 may load instructions from storage 4106 or another source (such as, for example, another computer system 4100) to memory 4104. Processor 4102 may then load the instructions from memory 4104 to an internal register or internal cache. To execute the instructions, processor 4102 may retrieve the instructions from the internal register or internal cache and decode them. During or after execution of the instructions, processor 4102 may write one or more results (which may be intermediate or final results) to the internal register or internal cache. Processor 4102 may then write one or more of those results to memory 4104. In particular embodiments, processor 4102 executes only instructions in one or more internal registers or internal caches or in memory 4104 (as opposed to storage 4106 or elsewhere) and operates only on data in one or more internal registers or internal caches or in memory 4104 (as opposed to storage 4106 or elsewhere).

One or more memory buses (which may each include an address bus and a data bus) may couple processor 4102 to memory 4104. Bus 4112 may include one or more memory buses, as described below. In particular embodiments, one or more memory management units (MMUs) reside between processor 4102 and memory 4104 and facilitate accesses to memory 4104 requested by processor 4102. In particular embodiments, memory 4104 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 4104 may include one or more memory devices 4104, where appropriate. Although this disclosure describes and illustrates particular memory, this disclosure contemplates any suitable memory.

In particular embodiments, storage 4106 includes mass storage for data or instructions. As an example, and not by way of limitation, storage 4106 may include a hard disk drive (HDD), a floppy disk drive, flash memory, an optical disc, a magneto-optical disc, magnetic tape, or a Universal Serial Bus (USB) drive or a combination of two or more of these. Storage 4106 may include removable or non-removable (or fixed) media, where appropriate. Storage 4106 may be internal or external to computer system 4100, where appropriate. In particular embodiments, storage 4106 is non-volatile, solid-state memory. In particular embodiments, storage 4106 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 4106 taking any suitable physical form. Storage 4106 may include one or more storage control units facilitating communication between processor 4102 and storage 4106, where appropriate. Where appropriate, storage 4106 may include one or more storages 4106. Although this disclosure describes and illustrates particular storage, this disclosure contemplates any suitable storage.

In particular embodiments, I/O interface 4108 includes hardware, software, or both, providing one or more interfaces for communication between computer system 4100 and one or more I/O devices. Computer system 4100 may include one or more of these I/O devices, where appropriate. One or more of these I/O devices may enable communication between a person and computer system 4100. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 4106 for them. Where appropriate, I/O interface 4108 may include one or more device or software drivers enabling processor 4102 to drive one or more of these I/O devices. I/O interface 4108 may include one or more I/O interfaces 4106, where appropriate. Although this disclosure describes and illustrates a particular I/O interface, this disclosure contemplates any suitable I/O interface.

In particular embodiments, communication interface 4110 includes hardware, software, or both providing one or more interfaces for communication (such as, for example, packet-based communication) between computer system 4100 and one or more other computer systems 41000 or one or more networks. As an example, and not by way of limitation, communication interface 4110 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 4110 for it.

As an example, and not by way of limitation, computer system 4100 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 4100 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 4100 may include any suitable communication interface 4110 for any of these networks, where appropriate. Communication interface 4110 may include one or more communication interfaces 4110, where appropriate. Although this disclosure describes and illustrates a particular communication interface, this disclosure contemplates any suitable communication interface.

In particular embodiments, bus 4112 includes hardware, software, or both coupling components of computer system 4100 to each other. As an example, and not by way of limitation, bus 4112 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 4112 may include one or more buses 4112, where appropriate. Although this disclosure describes and illustrates a particular bus, this disclosure contemplates any suitable bus or interconnect.

### 11. Experimental Results

### Example 1. Clinical Validation

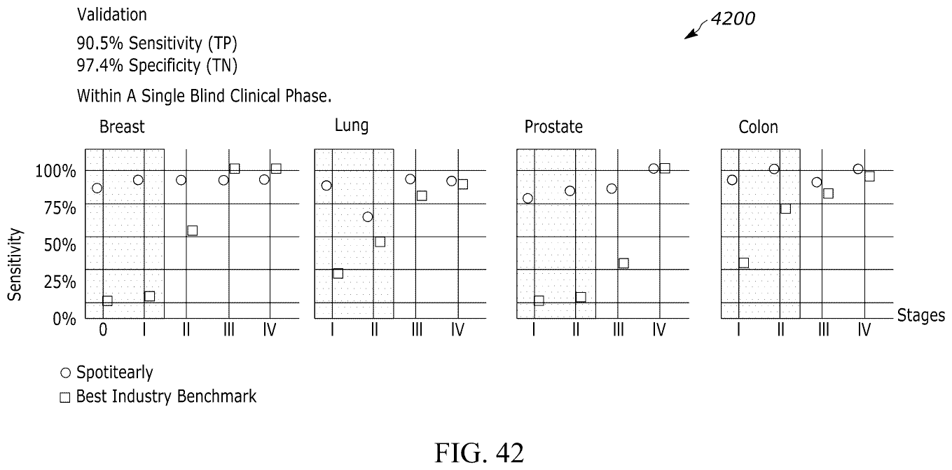

FIG. 42 illustrates data 4200 from a single blind clinical phase study which shows that the disclosed systems and methods have been validated by traditional cancer detection methods (e.g., a biopsy) and detects breast, lung, prostate, and colon cancers at similar or better rates compared to traditional industry benchmarks. For instance, the single blind clinical phase study indicated that the disclosed systems and methods have a 90.5% sensitivity rate and a 97.4% specificity rate.

### Example 2. Double-Blind Clinical Study

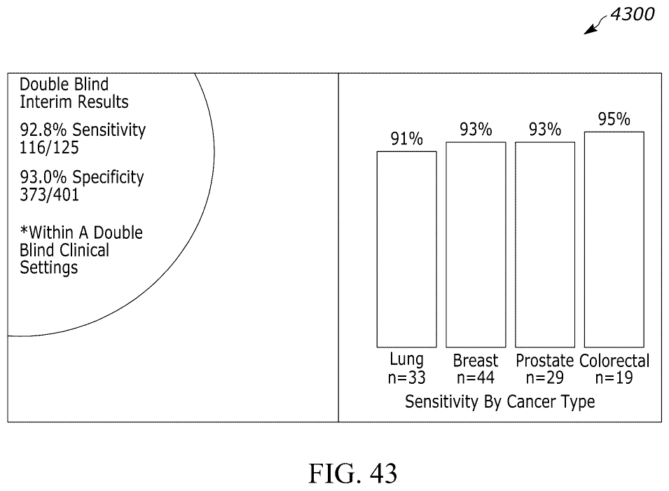

FIG. 43 illustrates mid-term results 4300 of a double-blind clinical study which was based on a sample of 575 participants that include verified cancer patients—some at a very early stage of the disease—and a control group verified as negative for cancer. The results indicate a 92.8% success rate in identifying the four most common types of cancer-breast, lung, colorectal, and prostate. The disclosed systems and methods show high sensitivity even for early stages, before the appearance of symptoms, which is critical for effective treatment of the disease and saving the patient's life. The data also indicate a low false identification percentage, on the order of 7%. The participants' samples were collected at the hospitals and sent for testing under fully blinded experiment conditions. The survey test was able to identify 92.8% of the sick participants (a particularly high sensitivity compared to the survey measures currently available in the world). The percentage of false positives for the mid-term results was 6.98% (i.e., a test specificity of 93.0%). The test showed stability across the four types of cancer represented in the study: breast cancer, 93%; lung cancer, 91%; colorectal cancer, 95%; and prostate cancer, 93%. Notably, unlike other screening tests that have recently come into use, the high specificity of the disclosed systems and methods here do not come at the expense of sensitivity.

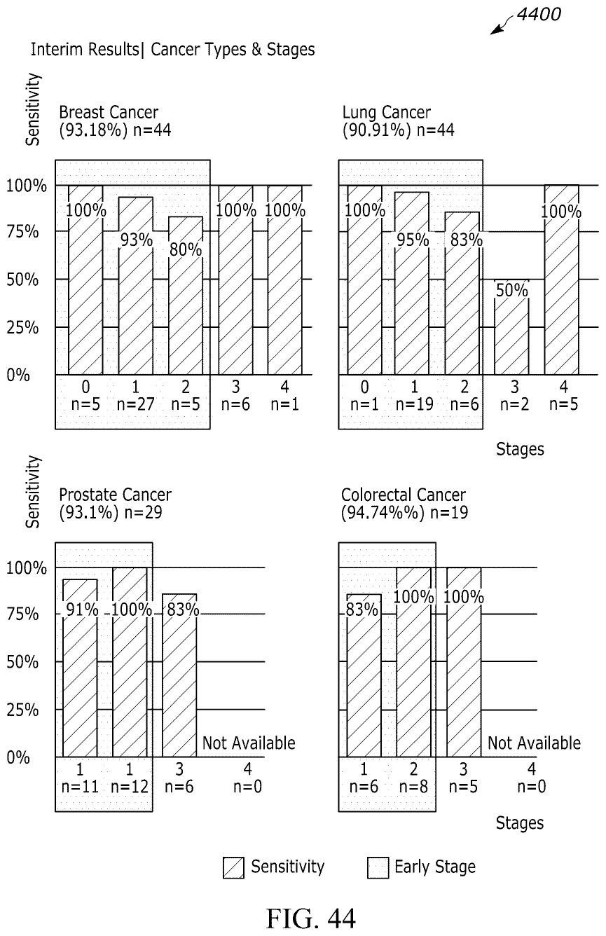

FIG. 44 illustrates the mid-term results 4400 of the double-blind clinical study based on cancer type and stages. The results are particularly encouraging in light of the fact that the level of test sensitivity remained high even in the early stages of the disease, when symptoms usually do not appear. Detection at these early stages may be critical for treatment effectiveness and success. The sensitivity of the test in stage 1 of the tumors was 93% for breast cancer, 95% for lung cancer, 91% for prostate cancer, and 83% for colorectal cancer.

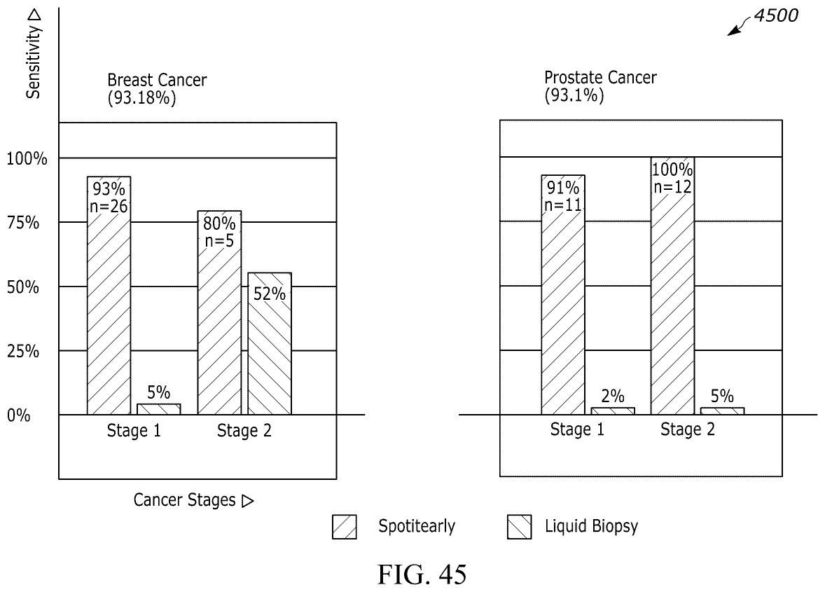

FIG. 45 illustrates mid-term results 4500 of the double-blind clinical study, and in particular, compares the sensitivity of the present systems and methods with that of a traditional liquid biopsy. The results are highly encouraging in light of the fact that for each type of cancer analyzed, the disclosed systems and methods had a higher sensitivity than a liquid biopsy test at both stage 1 and stage 2 cancer stages.

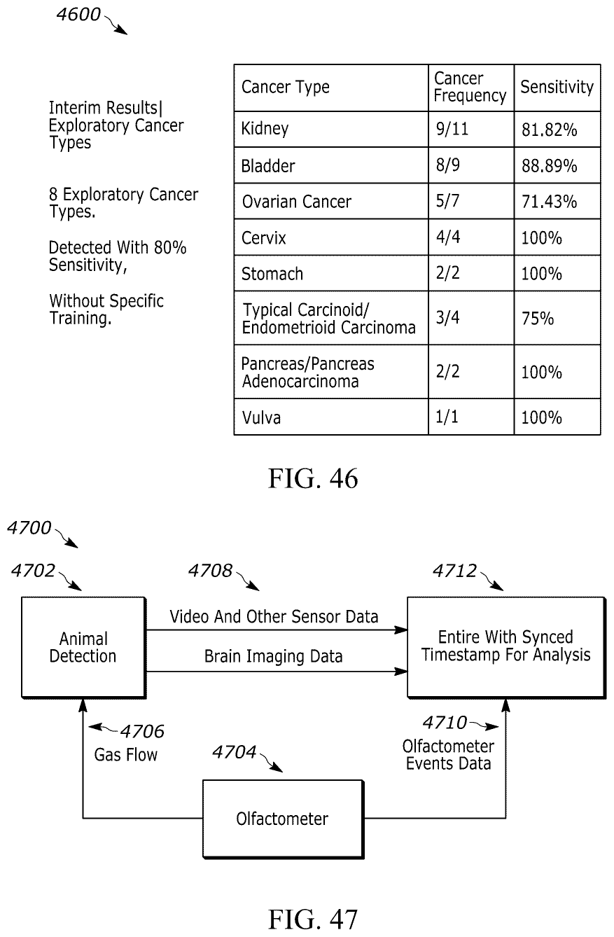

FIG. 46 illustrates mid-term results 4600 of the double-blind clinical study, and in particular, shows data for certain cancers which the detection animal was not specifically trained to detect. In this study, the detection animals were trained to detect breast, lung, prostate, and colorectal cancer. However, the detection animals also detected eight additional cancer types, including, kidney, bladder, ovarian, cervical, stomach, typical carcinoid/endometrial carcinoma, pancreatic/pancreas adenocarcinoma, and vulvar cancers.

FIG. 47 depicts an example method 4700 of utilizing brain imaging data for disease-detection. At animal step 4702, one or more detection animals wear a neurological sensor which is operable to gather brain imaging data. For example, the neurological sensor may be an EEG device comprising a plurality of electrodes worn by the detection animal. The animal detection step may further comprise behavioral sensors, such as an accelerometer or gyroscope worn by the detection animal, or an image or audio sensor placed in the test facility. In particular embodiments, the detection animal is exposed to a biological sample via an olfactometer at step 4704. The olfactometer delivers a gas sample to the detection animal, the gas sample comprising VOCs from the biological sample, at step 4706. Optionally, during a control run, the olfactometer delivers a gas sample comprising clean air. That is, the clean air cleans the flow paths and sampling port. Further, the clean air “re-calibrates” the detection animal by exposing it to an odorless gas. At step 4708, data, including behavioral sensor data, physiological sensor data, and neurological sensor data (e.g., brain imaging data) is streamed to a database. The olfactometer of step 4704 transmits, at step 4710, olfactometer events data. In particular embodiments, the olfactometer events data comprises one or more of a duration of sample exposure, and beginning time of sample exposure, and an ending time of sample exposure. At step 4712, data received from the video/other sensor data and the brain imaging data is synchronized with the olfactometer events data to form a complete timeline of events for analysis. In particular embodiments, data compiled at step 4712 is input into a neurological-based ML-model for disease-detection. Although this disclosure describes and illustrates particular steps of the method of FIG. 47 as occurring in a particular order, this disclosure contemplates any suitable steps of the method of FIG. 47 occurring in any suitable order. In particular embodiments, neurological testing of the detection animal is performed as a verification step of another test (e.g., a behavioral-based test). In particular embodiments, the verification step confirming the outputted disease state from a prior test. In particular embodiments, the verification step confirming the outputted disease state from a prior test and providing additional information, such as a cancer type or a cancer stage. In particular embodiments, neurological testing of the detection animal is performed as a standalone test capable of detecting one or more of: a cancer state (e.g., a positive or negative state), a cancer type, or a cancer stage.

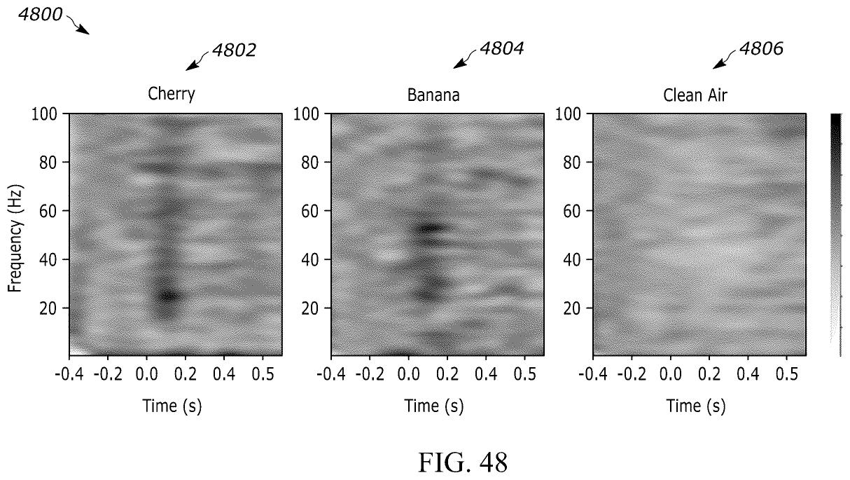

FIG. 48 depicts an example neurological data 4800 from a canine. The neurological data 4800 comprises the canine's responses to an odor of a cherry, banana, or clean air. Graph 4802 characterizes a neurological response to a cherry, graph 4804 characterizes a neurological response to a banana, and graph 4806 characterizes a neurological response to clean air.

Each response 4802, 4804, and 4806 is presented in the frequency domain in different timepoints, thereby reflecting both the frequency and the time domains. The graphs 4802, 4804, and 4806 are based on an aggregation of many exposures of the same sample in the same trial. Each exposure to a target sample (e.g., a cherry, banana, or patient sample) is an “epoch.” Between each epoch, clean air is flowed through the olfactometer system, thereby removing the odor from the tubes and recalibrating the canine's olfactory system. While the canine is exposed to the clean air, the EEG continues to record the brain activity, and therefore EEGs from this period reflect the brain activity in a resting state. The EEG data associated with the resting state is used as a baseline for brain activity during the odor exposure.

A detection animal exhibits a different neurological response when exposed to different odors. That is, different odors result in different power values for different frequencies of the EEG measurement as compared to a baseline frequencies' power values of the EEG measurement. The absolute frequency power is obtained from the EEG, and the graphs 4802, 4804, and 4806 present the power values in a representative way. For example: at freq=10 at timepoint 0.2 ms, the power is X. The data is visualized in the graph as:

$$\frac{X - Y}{Y}$$

wherein Y is the average power of freq=10 at the time range before the exposure (e.g., from −0.2 to 0).

Odor exposure occurs at time 0. The power values for each frequency at each time is calculated using Wavelet decomposition (e.g., a Morlet Wavelet).

In particular embodiments, neurological data presented in the manner described herein may be input into a neurological-based ML-model. In particular embodiments, the output of neurological-based ML-model may be input into a container comprising behavioral, physiological data, and/or patient data, wherein data from the container is input into a dog-specific ML-model. In particular embodiments, neurological-based ML-model may function as a standalone test capable of detecting one or more of: a cancer state (e.g., a positive or negative state), a cancer type, or a cancer stage.

### Example 3. Facial Gestures as Physiological Measures of Canine Response

Emotional facial expressions in dogs can communicate desires, intentions and may influence emotional state. The ability of dogs to accurately recognize and discriminate emotional information, including the facial emotions, promote adaptive behavior in response to perceived information. Research suggests that dogs' brains process, differentiate, and integrate multimodal sensory inputs of different emotional valence. Therefore, analysis of facial gestures displayed by dogs in response to an external stimulus, for example, exposure to an odor (biological sample) is a useful physiological measure for interpreting responses. Such analysis does not require pre-conditioning/training of the dogs and therefore, offer advantages over conditioned responses (e.g., sitting after exposure to a positive sample).

FIG. 29 depicts an example computing architecture for analyzing unconditioned facial gestures comprising a plurality of cameras for capturing facial gestures of the canines. The test facility for facial recognition is similar to those shown in FIGS. 14 and 15. In particular embodiments, when the biological sample is a breath sample, the testing room is equipped with cameras disposed near the sampling ports for capturing video images of facial features before, during, and after the dog is exposed to the sample. The video images from each camera is transferred to a computer (e.g., RPI), which is in communication with the LUCID platform over a cloud network. As an example, and not by way of limitation, the LUCID platform analyzes the eyes, face, or nose positions of the canine. As an example, and not by way of limitation, the eyes, face, or nose positions of the canine is indicative of whether the canine has detected a sample having a positive disease state or a negative disease state.

Table 1 shows an example summary comparison of performance characteristics using the AI algorithm model and the conditioned canine model (a canine round without the use of ML-based model) from multiple dogs, showing a significant benefit when using the AI model as illustrated by improved accuracy, specificity, and sensitivity in response prediction.

TABLE 1
Comparison of performance characteristics
for the AI model and canine model

| | Accuracy | Specificity | Sensitivity |
|---|---|---|---|
| Canine Model | 73.2 | 87.3 | 48.2 |
| (150 neg, 85 pos) | | | |
| Model AI | 86.5 | 93.3 | 76.2 |
| (24 neg, 11 pos) | | | |

TABLE 2
Comparison of performance characteristics
for the AI model and canine model

| | Accuracy | Specificity | Sensitivity |
|---|---|---|---|
| Dog “A” Model | 69.8 | 84.2 | 44.2 |
| (108 neg, 61 pos) | | | |
| Model AI | 85.2 | 81.6 | 91.9 |
| (20 neg, 10 pos) | | | |

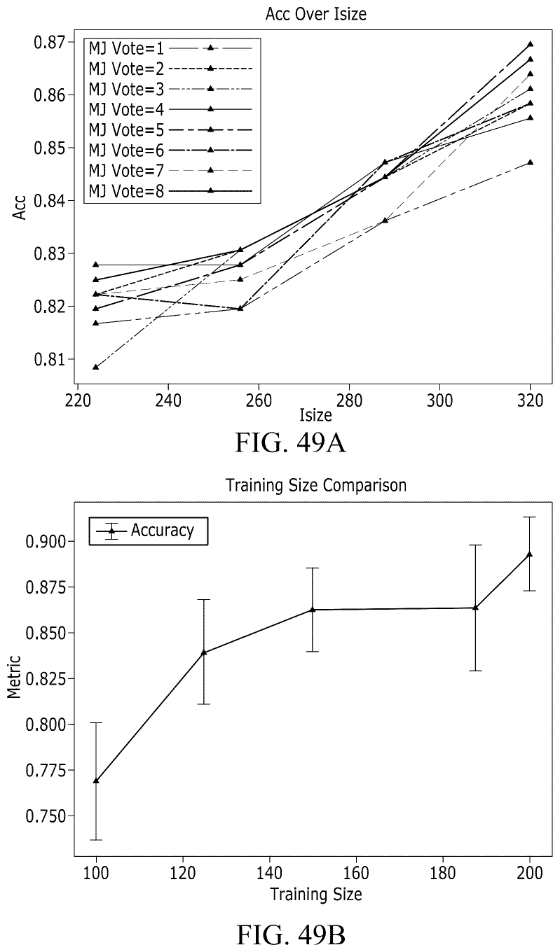

Table 2 shows a similar comparison of performance on a per dog basis. Further a comparison of accuracy vs sample size (e.g., increasing the number of frames/sec) and image resolution revealed improvement in accuracy with increasing sample size, increasing the image, and adding majority voting (“MJ Vote”) (FIGS. 49A-49B). Majority voting is a post-processing technique applied after classification, to make a smooth and reliable decision from a dense stream of class decisions.

FIGS. 49A-49B illustrate an example relationship between accuracy in detection, sample size and image. The data revealed improvement in accuracy with increasing sample size, increasing the image resolution and adding MJ Vote (FIGS. 49A-49B). These data demonstrate benefits of the unconditioned facial feature AI model over conditioned models as many behavioral noises are “cleaned up.”

### Example 4. Cancer Screening Using Trained Detection Dogs and Artificial Intelligence (AI): A double-blind study

### A. Materials and Methods

### A.1. Participants and Setting

This clinical trial study (NCT06255041) was conducted after approval by the local institutional ethics committees of Tel Aviv, Sourasky Medical Center and Rambam Health Care Campus. All breath samples in the study, including those for canine training, maintenance of canine detection abilities, and double-blind study, were provided after the participants gave their informed consent for collection, storage, distribution of samples, and data for use in future research studies before enrolling in the study.

The study population included males and females 18 years of age or older who attended extensive cancer screening at an integrated cancer prevention center (cancer screening arm) or underwent a biopsy for a suspected malignancy at the study sites (enriched arm). Samples were also obtained at Hadassah Medical Center from the Israel National Biobank for Research.

To prevent any confounders that might impact the VOCs collected in the breath test, participants were excluded from participation in the study if they had smoked less than two hours prior to providing the breath sample, or if they had coffee or an alcoholic beverage, or had eaten a meal less than one hour prior to providing the breath sample. Participants were also excluded if diagnosed and treated for cancer seven years prior to the study (except for non-metastatic skin tumors that were surgically removed), had chemotherapy in the last seven years, underwent a medical procedure in the thorax or airways two weeks prior to providing the breath sample, had an ongoing Helicobacter pylori infection or stomach ulcer, had an intestinal bowel disease flare or an ongoing active infection.

### A.2. Sample Collection and Processing

Participants were instructed to wear a surgical mask and inhale and exhale normally through the mouth for 5 minutes. The surgical mask was then sealed in two plastic bags and stored at room temperature. The samples were received at the laboratory and prepared for testing according to the laboratory operating protocols. Sample collection, delivery, and storage were managed according to validated laboratory work instructions designed to preserve sample quality.

The results of each participant's cancer screening tests or biopsy were recorded as negative or positive. Cancer type and stage, according to the American Joint Committee on Cancer (AJCC) cancer staging manual (29) were also recorded if the sample was positive. Laboratory personnel are blinded to the recorded results and any information that could reveal participants' clinical conditions until their codes were unblinded for analysis. Additionally, a random identification (ID) number was assigned to each sample to ensure that it was not correlated with any clinical confounders.

### A.3. Detection Canines

Six Labrador Retrievers were selected in accordance with the in-house canine selection protocol. The canines were individually housed in large kennels and maintained on a standard diet and water and regular exercise.

All canines were handled by professional canine trainers during canine training and double-blind testing sessions. Canines were trained to detect malignant lung, breast, colorectal, and prostate tumors. A total of 129 cancer-positive samples from all four cancer types and 340 cancer-negative samples were used for training the canines to detect these cancer types over a period of 6 months. The canines were trained to mark a sample as positive for cancer through a distinct behavioral cue-sitting beside the sample immediately after sniffing. Marking the sample as negative is done by continuing to the next sample without sitting. This action lasted for less than a second. To avoid bias, the samples used for training the canines were distinct from those used in the double-blind testing phase of the study. In addition, to maintain the canines' high performance level, maintenance training sessions were conducted throughout the clinical trial. These sessions utilized samples specifically designated for this purpose and were not included in the double-blind sample set. Training and maintenance were conducted in accordance with protocols developed in-house.

### A.4. Testing Room and Laboratory Monitoring System

The testing room (FIG. 15) included multiple portable sampling ports, each containing one sample at a time. The room was equipped with sensors and cameras that collect and stream in real-time, canine physical and behavioral data to a proprietary internal application. The activity in the testing room was monitored from a monitoring room. Real-time information gathering through such methods also helped identify for example, unusual canine behavior that may indicate inattention or hesitation, and alert the test manager for an appropriate response. In addition, the internal application monitored and visualized the test dynamics in real-time and maintained a record of the tests for post-test review.

### A.5. Cancer Detection by Using a First Bio-Hybrid AI Algorithm.

The bio-hybrid unit consists of two main AI components. The first component is an abnormal sniff predictive model that evaluates each sniff in real-time. This allows the test manager to continuously monitor canine performance and respond promptly as needed, in accordance with the laboratory's work instructions. The second component is a cancer prediction model, which provides a cancer prediction score for each sample immediately after the test, based on the canine's behavior and overall body language.

Each detection test comprised six double-blind samples with varying numbers (0-2) of cancer-positive samples. These six samples were evaluated by multiple bio-hybrid units, each consisting of a canine and AI tools. The dogs entered the testing room (FIG. 15) one by one to test all six samples that were loaded to the sampling ports. The canine sniffed each sample multiple times, while the AI tools analyzed the canine's conditioned response (sitting), in addition to behavioral and physiological unconditioned responses monitored by sensors and cameras in the detection room. This process yielded a cancer status prediction (positive or negative) for each sample.

Four bio-hybrid units operated sequentially and independently, each with a different canine. Afterward, the test result for each sample was determined based on the majority consensus among the four units. In the event of a tie, where two units predicted a positive sample and the other two predicted a negative sample, an additional unit was assigned to determine the final sample test result. The test result indicated the presence of cancer but did not specify the cancer type.

### A.6. Data Sources

The algorithms were trained using a combination of two sample sources: unblinded training samples and double-blind samples. The unblinded training samples were collected both before and during the double-blind phase of the study. The double-blind samples were added to the dataset once they were made available, following two unblinding points—the interim analysis and the end of the study.

### A.7. Data Pre-Processing

The dataset was partitioned into two subsets: a training set (70%) and a test set (30%). The test set, which was unseen during model training, was used to evaluate the performance of the trained model to avoid overestimation of performance due to training overfitting.

The features for the models were developed by the in-house data science team and canine research team, relying on canine behavioral literature (44-47). The features were collected using sensors and cameras deployed in the laboratory, capturing unconditional behavioral gestures of the canines in response to the sample.

### A.8. Model Framework and Evaluation

The selected model framework was the Gradient Boosting algorithm (48), which was implemented using the XGBoost package in Python (49). Gradient Boosting (50) has several beneficial features including, high accuracy, inherent feature selection ability and efficient handling of missing values.

The training process involved selecting two sets of parameters: hyperparameters, which define the model's structure, and model parameters, such as weights and thresholds. These parameters were chosen sequentially using the training data. First, the optimal hyperparameters were selected from a grid of potential sets of hyperparameter values by a grid-search cross-validation technique (51). Once the optimal hyperparameters were selected, the model was retrained on the entire training dataset using the selected hyperparameters. After training, the model's performance was assessed using the test data, which was not used in the training process and therefore could not enhance performance overestimation due to training overfitting.

### B. Outcome Measures

Each participant's sample test result was compared to its standard-of-care cancer screening or biopsy result. The primary outcome of the study was to estimate test sensitivity and specificity for the four types of malignancies that the canine were trained to detect (breast, lung, colorectal, and prostate). Sensitivity is defined as the number of samples that are correctly detected by the test as positive (true positive) out of all clinically positive samples. Specificity is defined as the number of samples that are correctly detected by the test as negative (true negative) out of all clinically negative samples.

Secondary outcome measures included the sensitivity of each of the four cancer types, and the sensitivity of early-stage detection (stages 0-2; stage 0 was only relevant for breast and lung cancer) both collectively and individually for each cancer type.

An exploratory outcome was the ability of the test (comprising the bio-hybrid unit) to detect other malignancies, in addition to the four it was trained to detect. Another exploratory outcome was the assessment of test repeatability using randomly selected samples that were tested twice using the same or different canines.

### C. Simulation of an Experimental Second Bio-Hybrid AI Algorithm

In addition to the bio-hybrid algorithm that was used to generate the test results, an experimental second algorithm (variant) was developed, using the same modeling framework described above. The second bio-hybrid algorithm incorporated additional data including participant demographics and medical history. The second bio-hybrid algorithm featured a model architecture that is different from the majority consensus approach used in the bio-hybrid AI described above. The second bio-hybrid algorithm utilized a distinct modeling layer to aggregate information from the bio-hybrid units in a more sophisticated manner. This aggregation efficiently weighted the bio-hybrid units, making the test results less susceptible to the natural variance in canine performance. Specifically, the cancer predictive model assigned a score to each sample, which was then compared to a predefined threshold. If the score exceeded the threshold, the sample was predicted as positive. Conversely, if the score was equal to or lower than the threshold, the sample was predicted as negative. Determination of the threshold aligned with product requirements, thereby balancing target sensitivity and specificity. The relationship between the threshold, sensitivity, and specificity was found to be pivotal. For example, an increased threshold value correlated with a decreased sensitivity and increased specificity, and vice versa. This relationship can be visualized by the receiver operating characteristic (ROC) curve. Evaluation of this model was conducted by computing the area under the curve (AUC). The second bio-hybrid algorithm was compared to the first bio-hybrid algorithm described in section 12.5, as well as to a simulated simple methodology without using AI tools, where cancer prediction is based solely on the conditional response of a single canine, that is, the test is positive if the canine sits next to the sample after sniffing it, whereas the test is negative if the canine does not sit next to it after sniffing it.

### D. Statistical Analysis

Test sensitivity and specificity measures were estimated along with their 95% confidence interval (CI) which was calculated using the Wilson score interval. The repeatability of the test was assessed by calculating the proportion of consistent results of randomly selected samples that were tested twice.

### E. Results

A total of 1386 participants (59.7% males) with a median age of 56 years (range 22-94 years) were included in the analysis. According to the medical centers' cancer screening/biopsy results, 1048 participants (75.6%) were negative for cancer and 338 (24.4%) were diagnosed with cancer (Table 3). Among the positive samples, 77 samples (5.6%) were positive for cancer types that the canines were not trained to detect. These samples included the following tumors: kidney, lower urinary tract, ovarian, carcinoid, cervical, endometrial, stomach, oropharyngeal space, vulvar, appendix, mesothelioma, thymoma, thyroid, and pancreatic cancer.

TABLE 3
Demographics and cancer screening/biopsy
results of the study participants

| | | Median | Males, | Females, | |
|---|---|---|---|---|---|
| Cancer screening/ | N | age | n | n | Current |
| biopsy results | (%) | (range) | (%) | (%) | smokers |
| Negative | 1048 | 54 | 664 | 384 | 97 |
| | | (22-85) | (63.4%) | (36.6%) | (9.2%) |
| Positive | 338 | | | | |
| Breast cancer | 80 | 60 | 1 | 79 | 6 |
| | | (28-92) | (1.3%) | (98.8%) | (7.5%) |
| Lung cancer | 80 | 70 | 34 | 46 | 20 |
| | | (47-88) | (42.5%) | (57.5%) | (25.0%) |
| Colorectal cancer | 30 | 66 | 20 | 10 | 9 |
| | | (42-92) | (66.7%) | (33.3%) | (30.0%) |
| Prostate cancer | 71 | 69 | 71 | 0 | 9 |
| | | (50-85) | (100%) | (0%) | (12.7%) |
| Other* | 77 | 67 | 37 | 40 | 13 |
| | | (28-94) | (48.0%) | (52.0%) | (16.9%) |

*Kidney, lower urinary tract, ovarian, carcinoid, cervical, endometrial, stomach, oropharyngeal space, vulvar, appendix, mesothelioma, thymoma, thyroid, and pancreatic cancer.

### E.1. Sensitivity and Specificity of Detection by Canines

The overall sensitivity and specificity of detection of the 4 cancer types that the canines were trained to detect were 93.9% (95% CI 90.3%-96.2%) and 94.3% (95% CI 92.7%-95.5%), respectively. The overall sensitivity of detection of all cancer types (including those that the canines were not trained to detect) was 91.1% (95% CI 87.6%-93.7%). The sensitivity values of each of the four cancer types were similar, ranging from 90.0% (95% CI 74.4%-96.5%) for colorectal cancer to 95.0% (95% CI 87.8%-98.0%) for lung and breast cancers (Table 4). The sensitivity for detection of other malignant tumors that the canines were not trained to detect is 81.8% (95% CI 71.8%-88.8%).

TABLE 4
Sensitivity and specificity of cancer detection by cancer type

| Cancer screening/biopsy | | Sensitivity (95% confidence |
|---|---|---|
| results | Frequency | interval) |
| All cancer types | 308/338 | 91.1% (87.6%-93.7%) |
| All 4 cancer types | 245/261 | 93.9% (90.3%-96.2%) |
| Breast cancer | 76/80 | 95.0% (87.8%-98.0%) |
| Lung cancer | 76/80 | 95.0% (87.8%-98.0%) |
| Colorectal cancer | 27/30 | 90.0% (74.4%-96.5%) |
| Prostate cancer | 66/71 | 93.0% (84.6%-97.0%) |
| Other* | 63/77 | 81.8% (71.8%-88.8%) |

*Kidney, lower urinary tract, ovarian, carcinoid, cervical, endometrial, stomach, oropharyngeal space, vulvar, appendix, mesothelioma, thymoma, thyroid, and pancreatic cancer

Table 5 shows the sensitivity results per cancer stage. All 9 samples with stage 0 (from breast and lung cancer) were detected (95% CI 70.1%-100%). In addition, stage 1 cancer was detected with a sensitivity of 94.9% (95% CI 89.8%-97.5%), and stage 2 cancer was detected with a sensitivity of 94.1% (95% CI 85.8%-97.7%). The overall sensitivity of early-stage cancer detection (stages 0-2) was 94.8% (95% CI 91.0%-97.1%).

TABLE 5
Sensitivity of cancer detection by stage

| Stage | Frequency | (95% confidence interval) | |
|---|---|---|---|
| 0* | 9/9 | 100.0% | (70.1%-100.0%) |
| I | 129/136 | 94.9% | (89.8%-97.5%) |
| II | 64/68 | 94.1% | (85.8%-97.7%) |
| 0-II* | 202/213 | 94.8% | (91.0%-97.1%) |
| III | 35/39 | 89.7% | (76.4%-95.9%) |
| IV | 8/9 | 88.9% | (56.5%-98.0%) |

*Only breast and lung malignancies were stage 0

The sensitivity of early-stage cancer detection per cancer type is shown in Table 6. As depicted in the table, the sensitivity values in early cancer stages closely resemble the overall sensitivity values for each respective cancer type. Moreover, despite early-stage positive samples constituting only a subset of the positive samples, substantial sample sizes exist for early-stage cancer detection across all types except for colorectal cancer. These ample sample sizes allow for precise estimation of early-stage sensitivity values with relatively small confidence intervals. Analysis of the sensitivity and specificity of cancer detection of the 4 cancer types that the canines were trained to detect by age group, gender and current smoking status showed similar values among the different categories (Table 7).

TABLE 6
Sensitivity of early-stage cancer detection by cancer type

| Type | Frequency | (95% confidence interval) |
|---|---|---|
| Breast | 61/65 | 93.8% (85.2%-97.6%) |
| Lung | 64/66 | 97.0% (89.6%-99.2%) |
| Colorectal | 19/22 | 86.4% (66.7%-95.3%) |
| Prostate | 58/60 | 96.7% (88.6%-99.1%) |

TABLE 7
Sensitivity of cancer detection by age,
gender, and current smoking status

| | Sensitivity* | Specificity* |
|---|---|---|
| Variable | n/N (%) | n/N (%) |
| Gender | | |
| Male | 116/126 (92.1%) | 624/664 (94.0%) |
| Female | 129/135 (95.6%) | 364/384 (94.8%) |
| Age category | | |
| ≤49 years | 26/28 (92.9%) | 390/412 (94.7%) |
| 50-64 years | 68/73 (93.2%) | 473/501 (94.4%) |
| ≥65 years | 151/160 (94.4%) | 125/135 (92.6%) |
| Smoking status | | |
| Current smoker | 39/44 (88.6%) | 90/97 (92.8%) |
| Non-smoker | 206/217 (94.9%) | 897/950 (94.4%) |

*Analysis included the four cancer types that the canines were trained to detect

### E.2. Assessment of Test Repeatability

The samples of 305 participants were tested twice to evaluate repeatability. Among these, 290 participants (95.1%) showed identical results both times (95% CI 92.0%-97.0%).

### E.3. Inference of the AI Predictive Models

The test results derived from the bio-hybrid algorithm used in the study were compared with the simulation outcomes of two alternative algorithms: a new variant of the bio-hybrid algorithm and a simple method where cancer prediction was based only on the conditional response of a canine-only method (without the use of AI tools). As shown in Table 8, performance outcomes decreased dramatically when AI was not used. Moreover, the simulated outcomes from the new bio-hybrid algorithm surpassed those of the currently used bio-hybrid algorithm. This outcome was expected, given the utilization of more comprehensive data and a more adaptable model architecture.

TABLE 8
Comparison of performance outcomes by prediction algorithm

| | Method | | |
|---|---|---|---|
| | Canine-only | Current bio-hybrid | New bio-hybrid |
| Metric | method | algorithm | algorithm |
| Sensitivity | 64.40% | 93.90% | 94.80% |
| Specificity | 93.90% | 94.30% | 96.20% |

FIG. 51B illustrates the ROC curve of the second bio-hybrid algorithm. The curve's trajectory demonstrates the tradeoff between sensitivity and specificity resulting from adjusting the model's threshold. The area under the curve (AUC) of the second bio-hybrid algorithm is 98.2%. In comparison, markers for the first bio-hybrid algorithm and the canine-only method lie below the ROC curve, suggesting a relatively lower prediction accuracy.

### F. Conclusion

The results of this study showed that the screening test described herein detects malignant lung, breast, colorectal, and prostate tumors in exhaled breath samples with 93.9% sensitivity and 94.3% specificity. Furthermore, the test demonstrates similar performance for early detection of cancer, with 94.8% sensitivity, a characteristic that sets it apart from many screening methods that are challenged by the need to maintain high sensitivity for early-stage cancer detection without compromising specificity. This is achieved by using the bio-hybrid AI approach, which allows balance between sensitivity and specificity by adjusting the model's prediction threshold. Moreover, the AI layer facilitates integration of additional data sources beyond canine responses to samples, including patient demographics and medical information.

Sensitivity of tests for the detection of each cancer type was comparable to the sensitivity of gold standard screening tests, e.g., low-dose computed tomography (9) for lung cancer, mammography with or without ultrasonography (10, 11) for breast cancer, fecal immunochemical tests (12) and colonoscopy (13) for colorectal cancer, and prostate-specific antigen (PSA) for prostate cancer (14). The AI based approach provides multitype cancer screening covering all four cancer types in a single test, thus demonstrating clinical utility.

In conclusion, this example introduces a scalable, low-cost multi-cancer screening method using a bio-hybrid approach of canines and AI that achieves high performance in early-stage detection using breath samples, within a setup that closely mirrors commercial-phase conditions. These features taken together with the improved sensitivity and specificity of cancer detection, specifically at early stages of cancer are superior and pave the way for development of a new generation of cancer screening tests with enhanced cancer screening capabilities.

### G. References Cited

1. Ferlay J, Ervik M, Lam F, Laversanne M, Colombet M, Mery L et al.; Global Cancer Observatory: Cancer Today Lyon, France: International Agency for Research on Cancer (2024).
2. Crosby D, Bhatia S, Brindle K M, Coussens L M, Dive C, Emberton M et al.; Early detection of cancer. Science 375: eaay9040 (2022).
3. Sasieni P, Smittenaar R, Hubbell E, Broggio J, Neal R D, Swanton C.; Modelled mortality benefits of multi-cancer early detection screening in England. Br J Cancer 129:72-80 (2023).
4. Henderson J T, Webber E M, Sawaya G F; Screening for Ovarian Cancer: Updated Evidence Report and Systematic Review for the US Preventive Services Task Force. JAMA 319:595-606 (2018).
5. Henrikson N B, Aiello Bowles E J, Blasi P R, Morrison C C, Nguyen M, Pillarisetty V G et al.; Screening for Pancreatic Cancer: Updated Evidence Report and Systematic Review for the US Preventive Services Task Force. JAMA 322:445-454 (2019).
6. Janfaza S, Khorsand B, Nikkhah M, Zahiri J.; Digging deeper into volatile organic compounds associated with cancer. Biol Methods Protoc 4: bpz014 (2019).
7. Monedeiro F, Dos Reis R B, Peria F M, Sares C T G, De Martinis B S; Investigation of sweat VOC profiles in assessment of cancer biomarkers using HS-GC-MS. J Breath Res 14:026009 (2020).
8. Deng C, Zhang X, Li N.; Investigation of volatile biomarkers in lung cancer blood using solid-phase microextraction and capillary gas chromatography-mass spectrometry. J Chromatogr B Analyt Technol Biomed Life Sci 808:269-277 (2004).
9. Wang C, Li P, Lian A, Sun B, Wang X, Guo L et al.; Blood volatile compounds as biomarkers for colorectal cancer. Cancer Biol Ther 15:200-206 (2014).
10. Xue R, Dong L, Zhang S, Deng C, Liu T, Wang J et al.; Investigation of volatile biomarkers in liver cancer blood using solid-phase microextraction and gas chromatography/mass spectrometry. Rapid Commun Mass Spectrom 22:1181-1186 (2008).
11. Monedeiro F, Monedeiro-Milanowski M, Zmysłowski H, De Martinis B S, Buszewski B; Evaluation of salivary VOC profile composition directed towards oral cancer and oral lesion assessment. Clin Oral Investig 25:4415-4430 (2021).
12. Silva C L, Passos M, Câmara J S; Investigation of urinary volatile organic metabolites as potential cancer biomarkers by solid-phase microextraction in combination with gas chromatography-mass spectrometry. Br J Cancer 105:1894-1904 (2011).
13. da Costa B R B, De Martinis B S; Analysis of urinary VOCs using mass spectrometric methods to diagnose cancer: A review. Clin Mass Spectrom 18:27-37 (2020).
14. Feil C, Staib F, Berger M R, Stein T, Schmidtmann I, Forster A et al Sniffer dogs can identify lung cancer patients from breath and urine samples. BMC Cancer 21:917 (2021).
15. Warli S M, Firsty N N, Velaro A J, Tala Z Z; The Olfaction Ability of Medical Detection Canine to Detect Prostate Cancer From Urine Samples: Progress Captured in Systematic Review and Meta-Analysis. World J Oncol 14:358-370 (2023).
16. Sonoda H, Kohnoe S, Yamazato T, Satoh Y, Morizono G, Shikata K et al.; Colorectal cancer screening with odour material by canine scent detection. Gut 60:814-819 (2011).
17. Ehmann R, Boedeker E, Friedrich U, Sagert J, Dippon J, Friedel G et al.; Canine scent detection in the diagnosis of lung cancer: revisiting a puzzling phenomenon. Eur Respir J 39:669-676 (2012).
18. McCulloch M, Jezierski T, Broffman M, Hubbard A, Turner K, Janecki T; Diagnostic accuracy of canine scent detection in early- and late-stage lung and breast cancers. Integr Cancer Ther 5:30-39 (2006).
19. Lippi G, Cervellin G.; Canine olfactory detection of cancer versus laboratory testing: myth or opportunity? Clin Chem Lab Med 50:435-439 (2012).
20. Horvath G, Jarverud G A, Jarverud S, Horvath I.; Human ovarian carcinomas detected by specific odor. Integr Cancer Ther 7:76-80 (2008).
21. McCulloch M, Turner K, Broffman M.; Lung cancer detection by canine scent: will there be a lab in the lab? Eur Respir J 39:511-512 (2012).
22. Bauer P, Leemans M, Audureau E, Gilbert C, Armal C, Fromantin I.; Remote Medical Scent Detection of Cancer and Infectious Diseases With Dogs and Rats: A Systematic Review. Integr Cancer Ther 21:15347354221140516 (2022).
23. Moser E, McCulloch M.; Canine scent detection of human cancers: A review of methods and accuracy. J Veterinary Behav 5:145-152 (2010).
24. Junqueira H, Quinn T A, Biringer R, Hussein M, Smeriglio C, Barrueto L et al.; Accuracy of Canine Scent Detection of Non-Small Cell Lung Cancer in Blood Serum. J Am Osteopath Assoc. (2019).
25. Pirrone F, Albertini M.; Olfactory detection of cancer by trained sniffer dogs: A systematic review of the literature. J Veterinary Behav 19:105-117 (2017).
26. Hackner K, Errhalt P, Mueller M R, Speiser M, Marzluf B A, Schulheim A et al.; Canine scent detection for the diagnosis of lung cancer in a screening-like situation. J Breath Res 10:046003 (2016).
27. Duan Y, Edwards J S, Dwivedi Y K; Artificial intelligence for decision making in the era of Big Data-evolution, challenges and research agenda. Int J Inf Manag 48:63-71 (2019).
28. Haleem A, Javaid M, Khan I H; Current status and applications of Artificial Intelligence (AI) in medical field: An overview. Curr Med Res Pract 9:231-237 (2019).
29. AJCC Cancer Staging Manual; 8 ed: Springer Cham (2017).
30. National Cancer Plan Bethesda, MD: National Cancer Institute At the National Institutes of Health (2024).
31. Shao S H, Allen B, Clement J, Chung G, Gao J, Hubbell E et al.; Multi-cancer early detection test sensitivity for cancers with and without current population-level screening options. Tumori 109:335-341 (2023).
32. Amundsen T, Sundstrøm S, BuvikT, Gederaas O A, Haaverstad R; Can dogs smell lung cancer? First study using exhaled breath and urine screening in unselected patients with suspected lung cancer. Acta Oncol 53:307-315 (2014).
33. Jonas D E, Reuland D S, Reddy S M, Nagle M, Clark S D, Weber R P et al.; Screening for Lung Cancer With Low-Dose Computed Tomography: Updated Evidence Report and Systematic Review for the US Preventive Services Task Force. JAMA 325:971-987 (2021).
34. Ohuchi N, Suzuki A, Sobue T, Kawai M, Yamamoto S, Zheng Y-F et al.; Sensitivity and specificity of mammography and adjunctive ultrasonography to screen for breast cancer in the Japan Strategic Anti-cancer Randomized Trial (J-START): a randomised controlled trial. Lancet 387:341-348 (2016).
35. Zeeshan M, Salam B, Khalid Q S B, Alam S, Sayani R; Diagnostic Accuracy of Digital Mammography in the Detection of Breast Cancer. Cureus 10: e2448 (2018).
36. Lee J K, Liles E G, Bent S, Levin T R, Corley D A; Accuracy of fecal immunochemical tests for colorectal cancer: systematic review and meta-analysis. Ann Intern Med 160:171 (2014).
37. Imperiale T F, Gruber R N, Stump T E, Emmett T W, Monahan P O; Performance Characteristics of Fecal Immunochemical Tests for Colorectal Cancer and Advanced Adenomatous Polyps: A Systematic Review and Meta-analysis. Ann Intern Med 170:319-329 (2019).
38. Merriel S W D, Pocock L, Gilbert E, Creavin S, Walter F M, Spencer A et al.; Systematic review and meta-analysis of the diagnostic accuracy of prostate-specific antigen (PSA) for the detection of prostate cancer in symptomatic patients. BMC Med 20:54 (2022).
39. Farooq M, Leevan E, Ahmed J, Ko B, Shin S, De Souza A et al.; Blood-based multi-cancer detection: A state-of-the-art update. Curr Probl Cancer 48:101059 (2024).
40. Klein E A, Richards D, Cohn A, Tummala M, Lapham R, Cosgrove D et al.; Clinical validation of a targeted methylation-based multi-cancer early detection test using an independent validation set. Ann Oncol 32:1167-1177 (2021).
41. Schrag D, Beer T M, McDonnell C H 3rd, Nadauld L, Dilaveri C A, Reid R et al.; Blood-based tests for multicancer early detection (PATHFINDER): a prospective cohort study. Lancet 402:1251-1260 (2023).
42. Nicholson B D, Oke J, Virdee P S, Harris D A, O'Doherty C, Park J E et al.; Multi-cancer early detection test in symptomatic patients referred for cancer investigation in England and Wales (SYMPLIFY): a large-scale, observational cohort study. Lancet Oncol 24:733-743 (2023).
43. Biehl W, Hattesohl A, Jörres R A, Duell T, Althöhn U, Koczulla A R et al.; VOC pattern recognition of lung cancer: a comparative evaluation of different dog- and eNose-based strategies using different sampling materials. Acta Oncol 58:1216-1224 (2019).
44. Siniscalchi, M., Lusito, R., Vallortigara, G. & Quaranta, A.; Seeing left- or right-asymmetric tail wagging produces different emotional responses in dogs. Curr Biol 23, 2279-2282 (2013).
45. Hasegawa, M., Ohtani, N. & Ohta, M.; Dogs' Body Language Relevant to Learning Achievement. Animals (Basel) 4, 45-58 (2014).
46. Siniscalchi, M., d′Ingeo, S., Minunno, M. & Quaranta, A. Communication in Dogs. Animals (Basel) 8 (2018).
47. Ferres, K., Schloesser, T. & Gloor, P. A.; Predicting Dog Emotions Based on Posture Analysis Using DeepLabCut. Future Internet 14, 97 (2022).
48. Friedman, J. H.; Greedy Function Approximation: A Gradient Boosting Machine. The Annals of Statistics 29, 1189-1232 (2001).
49. Chen, T. & Guestrin, C.; XGBoost: A Scalable Tree Boosting System. in KDD '16: Proceedings of the 22nd ACM SIGKDD International Conference on Knowledge Discovery and Data Mining 785-794 (2016).
50. Hastie, T., Tibshirani, R. & Friedman, J.; The Elements of Statistical Learning: Data Mining, Inference, and Prediction, Second Edition, (Springer, New York, NY, 2009).
51. Shekar, B. H. & Dagnew, G.; Grid Search-Based Hyperparameter Tuning and Classification of Microarray Cancer Data. in 2019 Second International Conference on Advanced Computational and Communication Paradigms (ICACCP) 1-8 (2019).

### 12. Recitation of Embodiments

Embodiment 1. A system for disease detection comprising: one or more machine learning-based (ML-based) disease-detection models trained on a dataset of detection events, wherein the models are operable to: receive a first sensor data associated with a first set of detection animals that have been exposed to a biological sample of a patient; calculate, based on the first sensor data, a first confidence score corresponding to a disease category associated with the biological sample, wherein the disease category comprises a plurality of disease states, and wherein the first confidence score indicates a likelihood of at least one of the disease states of the disease category being present in the patient; responsive to the first confidence score being greater than a first threshold score, receive a second sensor data associated with a second set of detection animals that have been exposed to the biological sample of the patient; and calculate, based on the second sensor data, one or more second confidence scores corresponding to one or more disease states in the disease category associated with the biological sample, wherein each confidence score indicates a likelihood of a respective disease state in the disease category being present in the patient.

Embodiment 2. The system of Embodiment 1, wherein: the first sensor data comprises data associated with a conditioned response of the first set of detection animals; and the second sensor data comprises data associated with a conditioned response of the second set of detection animals.

Embodiment 3. The system of Embodiment 2, wherein the first sensor data and the second sensor data comprise data received from one or more of: one or more behavioral sensors, one or more physiological sensors, or one or more environmental sensors.

Embodiment 4. The system of Embodiment 3, wherein the one or more behavioral sensors of the detection animal comprises one or more of: a face gesture of the detection animal, tail movements of the detection animal, landmarks on a skeleton model of the detection animal a duration of a sniff from the detection animal, a sniff intensity, a number of repeated sniffs, a pose of the detection animal, whether the detection animal looks at its handler, a pressure of a nose of the detection animal against a sampling port, or auditory features of the sniff.

Embodiment 5. The system of Embodiment 3, wherein the one or more physiological sensors comprises one or more of: one or more heart rate sensors, one or more heart rate variability sensors, one or more temperature sensors, one or more breath rate sensors, one or more sweat rate sensors, one or more blood pressure sensors, one or more skin temperature sensors, one or more pupil size variability sensors, one or more salivary cortisol sensors, one or more galvanic skin response (GSR) sensors, one or more electroencephalogram (EEG) sensors, one or more functional near-infrared spectroscopy (fNIR) sensors, one or more functional magnetic resonance imaging (fMRI) scanners, one or more electromyography imaging (EMG) scanners, or one or more magnetic resonance imaging (MRI) scanners.

Embodiment 6. The system of Embodiment 3, wherein the one or more environmental sensors comprise one or more of: one or more temperature sensors, one or more humidity sensors, one or more audio sensors, one or more gas sensors, or one or more air particulate sensors.

Embodiment 7. The system of Embodiment 1, wherein: the first set of detection animals is conditioned to detect the disease category of the biological sample; and the second set of detection animals is conditioned to detect the disease state of the biological sample.

Embodiment 8. The system of Embodiment 1, wherein: the system further comprises one or more breath sensors; and the models are further operable to: detect volatile organic compounds (VOCs) in the biological sample from the one or more breath sensors, wherein presence of the VOCs validates the biological sample as containing biological material from the patient.

Embodiment 9. The system of Embodiment 8, wherein the one or more breath sensors are selected from the group comprising: TVOC sensor, breath VOC sensor, relative humidity sensors, temperature sensor, photoionization detector (PID), flame ionization detector (FID), and metal oxide (MOX) sensor.

Embodiment 10. The system of Embodiment 1, wherein the models are further operable to: receive a third sensor data associated with the first set of detection animals or the second set of detection animals that have been exposed to one or more of a service sample, and calculate, based on the third sensor data, one or more confidence scores, each corresponding to a positive control category or a negative control category.

Embodiment 11. The system of Embodiment 10, wherein the first and second sets of detection animals are exposed to each of the biological sample and the service sample via a sampling port.

Embodiment 12. The system of Embodiment 11, wherein the sampling port is fluidly connected to one or more receptacles of a plurality of receptacles, each receptacle operable to hold the biological sample or the service sample.

Embodiment 13. The system of Embodiment 12, wherein the models are further operable to determine which of a particular sample to expose to the first set of detection animals or the second set of detection animals, wherein the particular sample is selected from a group consisting of: the biological sample from the patient and the service sample.

Embodiment 14. The system of Embodiment 1, wherein the models are further operable to: identify the biological sample as associated with at least one of the disease states of the disease category when the first confidence score is equal to or greater than a threshold value; or identify the biological sample as not associated with at least one of the disease states of the disease category when the first confidence score is less than the threshold value.

Embodiment 15. The system of Embodiment 1, wherein the models are further operable to: identify the biological sample as associated with the respective disease state in the disease category when the second confidence score is equal to or greater than a threshold value; or identify the biological sample as not associated with the respective disease state in the disease category when the second confidence score is less than the threshold value.

Embodiment 16. The system of Embodiment 13, wherein the respective disease state is identified with a sensitivity of at least approximately 90%.

Embodiment 17. The system of Embodiment 13, wherein the respective disease state is identified with a specificity of at least approximately 94%.

Embodiment 18. The system of Embodiment 1, wherein the biological sample is one or more of breath, saliva, urine, stool, skin emanations, tissue, or blood.

Embodiment 19. The system of Embodiment 1, wherein the disease category is selected from a group consisting of: cancer, liver disease, gastrointestinal disease, neurological disease, metabolic disease, vascular disease, and infectious disease.

Embodiment 20. The system of Embodiment 19, wherein the disease category is cancer, and the one or more disease states is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

Embodiment 21. A method of disease detection comprising: receiving a test kit, wherein the test kit comprises a biological sample from a patient; exposing the biological sample to a first set of detection animals; accessing a first sensor data associated with the first set detection animals; processing, using a first ML-based disease-detection model trained on a first dataset of detection events, the first sensor data to calculate a first confidence score corresponding to a disease category associated with the biological sample, wherein the disease category comprises a plurality of disease states, and wherein the first confidence score indicates a likelihood of at least one of the disease states of the disease category being present in the patient; responsive to the first confidence score being greater than a first threshold score, exposing the biological sample to a second set of detection animals; accessing a second sensor data associated with the second set of detection animals; and processing, using a second ML-based disease-detection model trained on a second dataset of detection events, the second sensor data to calculate one or more second confidence scores corresponding to one or more disease states in the disease category associated with the biological sample, wherein each confidence score indicates a likelihood of a respective disease state in the disease category being present in the patient.

Embodiment 22. The method of Embodiment 21, wherein: the first sensor data comprises data associated with a conditioned response of the first set of detection animals; and the second sensor data comprises data associated with a conditioned response of the second set of detection animals.

Embodiment 23. The method of Embodiment 21, further comprising: receiving a third sensor data associated with the first set of detection animals or the second set of detection animals that have been exposed to one or more of a service sample; and calculating, based on the third sensor data, one or more confidence scores, each corresponding to a positive control category or a negative control category.

Embodiment 24. The method of Embodiment 21, wherein the first and second sets of detection animals are exposed to each of the biological sample and the service sample via a sampling port.

Embodiment 25. The method of Embodiment 24, further comprising determining which of a particular sample to expose to the first set of detection animals or the second set of detection animals, wherein the particular sample is selected from a group consisting of: the biological sample from the patient and the service sample.

Embodiment 26. The method of Embodiment 21 further comprising: identifying the biological sample as associated with at least one of the disease states of the disease category when the first confidence score is equal to or greater than a threshold value; or identifying the biological sample as not associated with at least one of the disease states of the disease category when the first confidence score is less than the threshold value.

Embodiment 27. The method of Embodiment 21, further comprising: identifying the biological sample as associated with the respective disease state in the disease category when the second confidence score is equal to or greater than a threshold value; or identifying the biological sample as not associated with the respective disease state in the disease category when the second confidence score is less than the threshold value.

Embodiment 28. The method of Embodiment 27, wherein the respective disease state is identified with a sensitivity of at least approximately 90%.

Embodiment 29. The method of Embodiment 27, wherein the respective disease state is identified with a specificity of at least approximately 94%.

Embodiment 30. The method of Embodiment 21, wherein: the first set of detection animals is conditioned to detect the disease category of the biological sample; and the second set of detection animals is conditioned to detect the disease state of the biological sample.

Embodiment 31. The method of Embodiment 21, wherein the biological sample is one or more of breath, saliva, urine, stool, skin emanations, tissue, or blood.

Embodiment 32. The method of Embodiment 21, wherein the disease category is selected from a group consisting of: cancer, liver disease, gastrointestinal disease, neurological disease, metabolic disease, vascular disease, and infectious disease.

Embodiment 33. The method of Embodiment 32, wherein the disease category is cancer, and the one or more disease states is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

Embodiment 34. A method of disease detection comprising: receiving a test kit, wherein the test kit comprises a biological sample from a patient; exposing the biological sample to a first set of detection animals; accessing a first sensor data associated with each detection animal in the first set of detection animals, processing, using a first ML-based disease-detection model trained on a first dataset of detection events, the first sensor data associated with each detection animal in the first set of detection animals to determine whether the detection animal in the first set of detection animals indicate a disease category to present in the biological sample; in response to a determination that less than a first threshold percentage of the first set of detection animals indicate the disease category to be present in the biological sample, identifying the biological sample as not associated with the disease category; in response to a determination that between the first threshold percentage and a second threshold percentage of the first set of detection animals indicate the disease category to be present in the biological sample, exposing the biological sample to a subset of detection animals from the second set of detection animals; wherein: in response to a determination that less than a threshold fraction of the subset indicated a disease state to be present in the biological sample, identifying the biological sample as not associated with the disease category; and in response to a determination that greater than the threshold fraction of the subset indicated the disease state to be present in the biological sample, identifying the biological sample as requiring exposure to the second set of detection animals; and in response to a determination that greater than the second threshold percentage of the first set of detection animals indicate the disease category to be present in the biological sample, identifying the biological sample as requiring exposure to the second set of detection animals.

Embodiment 35. The method of Embodiment 34, further comprising: exposing the biological sample to a second set of detection animals; accessing a second sensor data associated with each detection animal in the second set of detection animals, processing, using a second ML-based disease-detection model trained on a second dataset of detection events, the second sensor data associated with each detection animal in the second set of detection animals to determine whether the detection animal in the second set of detection animals indicate the disease state to present in the biological sample; in response to a determination that less than a third threshold percentage of the second set of detection animals indicate the disease state to be present in the biological sample, identifying the biological sample as not associated with the disease state; and in response to a determination that greater than the third threshold percentage of the second set of detection animals indicate the disease state to be present in the biological sample, identifying the biological sample as associated with the disease state.

Embodiment 36. A method for determining a progression of a disease in a patient undergoing a treatment comprising: accessing patient data indicating the patient previously tested positive for a first disease state in a disease category and has subsequently received treatment for the disease; receiving a new test kit at a time after the patient has received the treatment for the disease, wherein the new test kit comprises a new biological sample from the patient; exposing the new biological sample to a set of detection animals; identifying the new biological sample as being associated with a second disease state; comparing the second disease state with the first disease state; and determining the progression of the disease in the patient after the treatment based on the comparing.

Embodiment 37. The method of Embodiment 36, further comprising, accessing a second sensor data associated with the set of detection animals; and processing, using a first ML-based disease-detection model trained on a first dataset of detection events, the second sensor data to calculate a second confidence score corresponding to the second disease state associated with the new biological sample.

Embodiment 38. The method of Embodiment 36, wherein the method further comprises, prior to accessing the patient data: receiving a prior test kit, wherein the prior test kit comprises a prior biological sample from the patient; exposing the prior biological sample to a set of detection animals; accessing a first sensor data associated with the set of detection animals; processing, using a first ML-based disease-detection model trained on a first dataset of detection events, the first sensor data to calculate a first confidence score corresponding to the first disease state associated with the prior biological sample; and identify the biological sample as associated with the first disease state when the first confidence score is equal to or greater than a threshold value.

Embodiment 39. The method of Embodiment 38, wherein: the new biological sample is one or more of breath, saliva, urine, stool, skin emanations, tissue, or blood; and the prior biological sample is one or more of breath, saliva, urine, stool, skin emanations, tissue, or blood.

Embodiment 40. The method of Embodiment 39, wherein the prior biological sample and the new biological sample are of a same sample type.

Embodiment 41. The method of Embodiment 36, wherein the disease category is selected from a group consisting of: cancer, liver disease, gastrointestinal disease, neurological disease, metabolic disease, vascular disease, and infectious disease.

Embodiment 42. The method of Embodiment 41, wherein the disease category is cancer, and the disease state is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

Embodiment 43. A method for training a detection animal to provide a conditioned response to be used with a machine learning-based (ML-based) disease-detection system comprising steps of: exposing a detection animal to a first biological sample from a subject having a target disease state; training the detection animal to provide the conditioned response by providing the detection animal with a reward for identifying the target disease state; inputting, to the disease-detection system, a first sensor data corresponding to the detection animal, wherein the first sensor data is associated with presence of the target disease state; storing tangibly, in a memory of a computer processor, the first sensor data to obtain a dataset of detection events; and training the ML-based disease-detection system to detect the disease state based on the dataset of detection events.

Embodiment 44. The method of Embodiment 43, wherein the conditioned response comprises a body pose of the detection animal.

Embodiment 45. The method of Embodiment 43, further comprising repeating each of the steps until a threshold sensitivity is reached by the detection animal.

Embodiment 46. The method of Embodiment 43, further comprising repeating each of the steps until a threshold specificity is reached by the detection animal.

Embodiment 47. The method of Embodiment 43, wherein the disease state is from a disease category selected from a group consisting of: cancer, liver disease, gastrointestinal disease, neurological disease, metabolic disease, vascular disease, and infectious disease.

Embodiment 48. The method of Embodiment 45, wherein the disease state is selected from a group consisting of: breast cancer, lung cancer, prostate cancer, brain cancer, bladder cancer, ovarian cancer, skin cancer, colorectal cancer, kidney cancer, lower urinary tract cancer, a carcinoid, pancreatic cancer, cervical cancer, endometrial cancer, vulvar cancer, stomach cancer, oropharyngeal cancer, appendicular cancer, mesothelioma cancer, thymoma cancer, and thyroid cancer.

Embodiment 49. The method of Embodiment 43, wherein the first biological sample and second biological sample are one or more of breath, saliva, urine, stool, skin emanations, tissue, or blood.

### 13. Miscellaneous

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to the disclosed systems and methods are in particular disclosed in the attached claims directed to a method, a storage medium, a system, and a computer program product, wherein any feature mentioned in one claim category, e.g., method, can be claimed in another claim category, e.g. system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A system for disease detection comprising:
one or more machine learning-based (ML-based) disease-detection models trained on a dataset of detection events, wherein the models are operable to:
receive a first sensor data associated with a first set of detection animals that have been exposed to a biological sample of a patient;
calculate, based on the first sensor data, a first confidence score corresponding to a disease category associated with the biological sample, wherein the disease category comprises a plurality of disease states, and wherein the first confidence score indicates a likelihood of at least one of the disease states of the disease category being present in the patient;
responsive to the first confidence score being greater than a first threshold score, receive a second sensor data associated with a second set of detection animals that have been exposed to the biological sample of the patient; and
calculate, based on the second sensor data, one or more second confidence scores corresponding to one or more disease states in the disease category associated with the biological sample, wherein each confidence score indicates a likelihood of a respective disease state in the disease category being present in the patient.

2. The system of claim 1, wherein:
the first sensor data comprises data associated with a conditioned response of the first set of detection animals; and
the second sensor data comprises data associated with a conditioned response of the second set of detection animals.

3. The system of claim 2, wherein the first sensor data and the second sensor data comprise data received from one or more of:
one or more behavioral sensors,
one or more physiological sensors, or
one or more environmental sensors.

4. The system of claim 3, wherein the one or more behavioral sensors of the detection animal comprises one or more of:
a face gesture of the detection animal,
tail movements of the detection animal,
landmarks on a skeleton model of the detection animal
a duration of a sniff from the detection animal,
a sniff intensity,
a number of repeated sniffs,
a pose of the detection animal,
whether the detection animal looks at its handler,
a pressure of a nose of the detection animal against a sampling port, or
auditory features of the sniff.

5. The system of claim 3, wherein the one or more physiological sensors comprises one or more of:
one or more heart rate sensors,
one or more heart rate variability sensors,
one or more temperature sensors,
one or more breath rate sensors,
one or more sweat rate sensors,
one or more blood pressure sensors,
one or more skin temperature sensors,
one or more pupil size variability sensors,
one or more salivary cortisol sensors,
one or more galvanic skin response (GSR) sensors,
one or more electroencephalogram (EEG) sensors,
one or more functional near-infrared spectroscopy (fNIR) sensors,
one or more functional magnetic resonance imaging (fMRI) scanners,
one or more electromyography imaging (EMG) scanners, or
one or more magnetic resonance imaging (MRI) scanners.

6. The system of claim 3, wherein the one or more environmental sensors comprise one or more of:
one or more temperature sensors,
one or more humidity sensors,
one or more audio sensors,
one or more gas sensors, or
one or more air particulate sensors.

7. The system of claim 1, wherein:
the first set of detection animals is conditioned to detect the disease category of the biological sample; and
the second set of detection animals is conditioned to detect the disease state of the biological sample.

8. The system of claim 1, wherein:
the system further comprises one or more breath sensors; and
the models are further operable to:
detect volatile organic compounds (VOCs) in the biological sample from the one or more breath sensors, wherein presence of the VOCs validates the biological sample as containing biological material from the patient.

9. The system of claim 8, wherein the one or more breath sensors are selected from the group comprising: TVOC sensor, breath VOC sensor, relative humidity sensors, temperature sensor, photoionization detector (PID), flame ionization detector (FID), and metal oxide (MOX) sensor.

10. The system of claim 1, wherein the models are further operable to:
receive a third sensor data associated with the first set of detection animals or the second set of detection animals that have been exposed to one or more of a service sample, and
calculate, based on the third sensor data, one or more confidence scores, each corresponding to a positive control category or a negative control category.

11. The system of claim 10, wherein the first and second sets of detection animals are exposed to each of the biological sample and the service sample via a sampling port.

12. The system of claim 11, wherein the sampling port is fluidly connected to one or more receptacles of a plurality of receptacles, each receptacle operable to hold the biological sample or the service sample.

13. The system of claim 12, wherein the models are further operable to determine which of a particular sample to expose to the first set of detection animals or the second set of detection animals, wherein the particular sample is selected from a group consisting of: the biological sample from the patient and the service sample.

14. The system of claim 1, wherein the models are further operable to:
identify the biological sample as associated with at least one of the disease states of the disease category when the first confidence score is equal to or greater than a threshold value; or
identify the biological sample as not associated with at least one of the disease states of the disease category when the first confidence score is less than the threshold value.

15. The system of claim 1, wherein the models are further operable to:
identify the biological sample as associated with the respective disease state in the disease category when the second confidence score is equal to or greater than a threshold value; or
identify the biological sample as not associated with the respective disease state in the disease category when the second confidence score is less than the threshold value.

16. The system of claim 13, wherein the respective disease state is identified with a sensitivity of at least approximately 90%.

17. The system of claim 13, wherein the respective disease state is identified with a specificity of at least approximately 94%.

18. The system of claim 1, wherein the biological sample is one or more of breath, saliva, urine, stool, skin emanations, tissue, or blood.

19. The system of claim 1, wherein the disease category is selected from a group consisting of:
cancer,
liver disease,
gastrointestinal disease,
neurological disease,
metabolic disease,
vascular disease, and
infectious disease.

20. The system of claim 19, wherein the disease category is cancer, and the one or more disease states is selected from a group consisting of:
breast cancer,
lung cancer,
prostate cancer,
brain cancer,
bladder cancer,
ovarian cancer,
skin cancer,
colorectal cancer,
kidney cancer,
lower urinary tract cancer,
a carcinoid,
pancreatic cancer,
cervical cancer,
endometrial cancer,
vulvar cancer,
stomach cancer,
oropharyngeal cancer,
appendicular cancer,
mesothelioma cancer,
thymoma cancer, and
thyroid cancer.

21. A method of disease detection comprising:
receiving a test kit, wherein the test kit comprises a biological sample from a patient;
exposing the biological sample to a first set of detection animals;
accessing a first sensor data associated with the first set detection animals;
processing, using a first ML-based disease-detection model trained on a first dataset of detection events, the first sensor data to calculate a first confidence score corresponding to a disease category associated with the biological sample, wherein the disease category comprises a plurality of disease states, and wherein the first confidence score indicates a likelihood of at least one of the disease states of the disease category being present in the patient;
responsive to the first confidence score being greater than a first threshold score, exposing the biological sample to a second set of detection animals;
accessing a second sensor data associated with the second set of detection animals; and
processing, using a second ML-based disease-detection model trained on a second dataset of detection events, the second sensor data to calculate one or more second confidence scores corresponding to one or more disease states in the disease category associated with the biological sample, wherein each confidence score indicates a likelihood of a respective disease state in the disease category being present in the patient.

22. The method of claim 21, wherein:
the first sensor data comprises data associated with a conditioned response of the first set of detection animals; and
the second sensor data comprises data associated with a conditioned response of the second set of detection animals.

23. The method of claim 21, further comprising:
receiving a third sensor data associated with the first set of detection animals or the second set of detection animals that have been exposed to one or more of a service sample; and
calculating, based on the third sensor data, one or more confidence scores, each corresponding to a positive control category or a negative control category.

24. The method of claim 23, wherein the first and second sets of detection animals are exposed to each of the biological sample and the service sample via a sampling port.

25. The method of claim 24, further comprising determining which of a particular sample to expose to the first set of detection animals or the second set of detection animals, wherein the particular sample is selected from a group consisting of: the biological sample from the patient and the service sample.

26. The method of claim 21 further comprising:
identifying the biological sample as associated with at least one of the disease states of the disease category when the first confidence score is equal to or greater than a threshold value; or
identifying the biological sample as not associated with at least one of the disease states of the disease category when the first confidence score is less than the threshold value.

27. The method of claim 21, further comprising:
identifying the biological sample as associated with the respective disease state in the disease category when the second confidence score is equal to or greater than a threshold value; or
identifying the biological sample as not associated with the respective disease state in the disease category when the second confidence score is less than the threshold value.

28. The method of claim 27, wherein the respective disease state is identified with a sensitivity of at least approximately 90%.

29. The method of claim 27, wherein the respective disease state is identified with a specificity of at least approximately 94%.

30. The method of claim 21, wherein:
the first set of detection animals is conditioned to detect the disease category of the biological sample; and
the second set of detection animals is conditioned to detect the disease state of the biological sample.

31. The method of claim 21, wherein the biological sample is one or more of breath, saliva, urine, stool, skin emanations, tissue, or blood.

32. The method of claim 21, wherein the disease category is selected from a group consisting of:
cancer,
liver disease,
gastrointestinal disease,
neurological disease,
metabolic disease,
vascular disease, and
infectious disease.

33. The method of claim 32, wherein the disease category is cancer, and the one or more disease states is selected from a group consisting of:
breast cancer,
lung cancer,
prostate cancer,
brain cancer,
bladder cancer,
ovarian cancer,
skin cancer,
colorectal cancer,
kidney cancer,
lower urinary tract cancer,
a carcinoid,
pancreatic cancer,
cervical cancer,
endometrial cancer,
vulvar cancer,
stomach cancer,
oropharyngeal cancer,
appendicular cancer,
mesothelioma cancer,
thymoma cancer, and
thyroid cancer.

34. A method of disease detection comprising:
receiving a test kit, wherein the test kit comprises a biological sample from a patient;
exposing the biological sample to a first set of detection animals;
accessing a first sensor data associated with each detection animal in the first set of detection animals,
processing, using a first ML-based disease-detection model trained on a first dataset of detection events, the first sensor data associated with each detection animal in the first set of detection animals to determine whether the detection animal in the first set of detection animals indicate a disease category to present in the biological sample;
in response to a determination that less than a first threshold percentage of the first set of detection animals indicate the disease category to be present in the biological sample, identifying the biological sample as not associated with the disease category;
in response to a determination that between the first threshold percentage and a second threshold percentage of the first set of detection animals indicate the disease category to be present in the biological sample, exposing the biological sample to a subset of detection animals from the second set of detection animals; wherein:
in response to a determination that less than a threshold fraction of the subset indicated a disease state to be present in the biological sample, identifying the biological sample as not associated with the disease category; and
in response to a determination that greater than the threshold fraction of the subset indicated the disease state to be present in the biological sample, identifying the biological sample as requiring exposure to the second set of detection animals; and
in response to a determination that greater than the second threshold percentage of the first set of detection animals indicate the disease category to be present in the biological sample, identifying the biological sample as requiring exposure to the second set of detection animals.

35. The method of claim 34, further comprising:
exposing the biological sample to a second set of detection animals;
accessing a second sensor data associated with each detection animal in the second set of detection animals,
processing, using a second ML-based disease-detection model trained on a second dataset of detection events, the second sensor data associated with each detection animal in the second set of detection animals to determine whether the detection animal in the second set of detection animals indicate the disease state to present in the biological sample;
in response to a determination that less than a third threshold percentage of the second set of detection animals indicate the disease state to be present in the biological sample, identifying the biological sample as not associated with the disease state; and
in response to a determination that greater than the third threshold percentage of the second set of detection animals indicate the disease state to be present in the biological sample, identifying the biological sample as associated with the disease state.

36. A method for determining a progression of a disease in a patient undergoing a treatment comprising:
accessing patient data indicating the patient previously tested positive for a first disease state in a disease category and has subsequently received treatment for the disease;
receiving a new test kit at a time after the patient has received the treatment for the disease, wherein the new test kit comprises a new biological sample from the patient;
exposing the new biological sample to a set of detection animals;
identifying the new biological sample as being associated with a second disease state;
comparing the second disease state with the first disease state; and
determining the progression of the disease in the patient after the treatment based on the comparing.

37. The method of claim 36, further comprising,
accessing a second sensor data associated with the set of detection animals; and
processing, using a first ML-based disease-detection model trained on a first dataset of detection events, the second sensor data to calculate a second confidence score corresponding to the second disease state associated with the new biological sample.

38. The method of claim 36, wherein the method further comprises, prior to accessing the patient data:
receiving a prior test kit, wherein the prior test kit comprises a prior biological sample from the patient;
exposing the prior biological sample to a set of detection animals;
accessing a first sensor data associated with the set of detection animals;
processing, using a first ML-based disease-detection model trained on a first dataset of detection events, the first sensor data to calculate a first confidence score corresponding to the first disease state associated with the prior biological sample; and
identify the biological sample as associated with the first disease state when the first confidence score is equal to or greater than a threshold value.

39. The method of claim 38, wherein:
the new biological sample is one or more of breath, saliva, urine, stool, skin emanations, tissue, or blood; and
the prior biological sample is one or more of breath, saliva, urine, stool, skin emanations, tissue, or blood.

40. The method of claim 39, wherein the prior biological sample and the new biological sample are of a same sample type.

41. The method of claim 36, wherein the disease category is selected from a group consisting of:
cancer,
liver disease,
gastrointestinal disease,
neurological disease,
metabolic disease,
vascular disease, and
infectious disease.

42. The method of claim 41, wherein the disease category is cancer, and the disease state is selected from a group consisting of:
breast cancer,
lung cancer,
prostate cancer,
brain cancer,
bladder cancer,
ovarian cancer,
skin cancer,
colorectal cancer,
kidney cancer,
lower urinary tract cancer,
a carcinoid,
pancreatic cancer,
cervical cancer,
endometrial cancer,
vulvar cancer,
stomach cancer,
oropharyngeal cancer,
appendicular cancer,
mesothelioma cancer,
thymoma cancer, and
thyroid cancer.

43. A method for training a detection animal to provide a conditioned response to be used with a machine learning-based (ML-based) disease-detection system comprising steps of:
exposing a detection animal to a first biological sample from a subject having a target disease state;
training the detection animal to provide the conditioned response by providing the detection animal with a reward for identifying the target disease state;
inputting, to the disease-detection system, a first sensor data corresponding to the detection animal, wherein the first sensor data is associated with presence of the target disease state;
storing tangibly, in a memory of a computer processor, the first sensor data to obtain a dataset of detection events; and
training the ML-based disease-detection system to detect the disease state based on the dataset of detection events.

44. The method of claim 43, wherein the conditioned response comprises a body pose of the detection animal.

45. The method of claim 43, further comprising repeating each of the steps until a threshold sensitivity is reached by the detection animal.

46. The method of claim 43, further comprising repeating each of the steps until a threshold specificity is reached by the detection animal.

47. The method of claim 43, wherein the disease state is from a disease category selected from a group consisting of:
cancer,
liver disease,
gastrointestinal disease,
neurological disease,
metabolic disease,
vascular disease, and
infectious disease.

48. The method of claim 45, wherein the disease state is selected from a group consisting of:
breast cancer,
lung cancer,
prostate cancer,
brain cancer,
bladder cancer,
ovarian cancer,
skin cancer,
colorectal cancer,
kidney cancer,
lower urinary tract cancer,
a carcinoid,
pancreatic cancer,
cervical cancer,
endometrial cancer,
vulvar cancer,
stomach cancer,
oropharyngeal cancer,
appendicular cancer,
mesothelioma cancer,
thymoma cancer, and
thyroid cancer.

49. The method of claim 43, wherein the first biological sample and second biological sample are one or more of breath, saliva, urine, stool, skin emanations, tissue, or blood.
